(19) United States
(12) Patent Application Publication
(10) Pub. No.: US 2024/0317718 A1
(43) Pub. Date: Sep. 26, 2024

(54) SUBSTITUTED HETEROCYCLIC COMPOUNDS

(71) Applicant: BRISTOL-MYERS SQUIBB COMPANY, PRINCETON, NJ (US)

(72) Inventors: STEVEN H. SPERGEL, WARRINGTON, PA (US); RYAN M. MOSLIN, PRINCETON, NJ (US); MICHAEL EDWARD MERTZMAN, NEW HOPE, PA (US); SHOSHANA L. POSY, HIGHLAND PARK, NJ (US); SIRISH KAUSHIK LAKKARAJU, PENNINGTON, NJ (US); JOSEPH A. TINO, LAWRENCEVILLE, NJ (US); ZILI XIAO, EAST WINDSOR, NJ (US); CHUNJIAN LIU, PENNINGTON, NJ (US); JAMES LIN, LAWRENCEVILLE, NJ (US)

(73) Assignee: BRISTOL-MYERS SQUIBB COMPANY, PRINCETON, NJ (US)

(21) Appl. No.: 18/560,485

(22) PCT Filed: May 13, 2022
(86) PCT No.: PCT/US2022/029105

§ 371 Date: Nov. 13, 2023

Related U.S. Application Data
(60) Provisional application No. 63/188,498, filed on May 14, 2021.
(60) Provisional application No. 63/328,835, filed on Apr. 8, 2022.

(51) Int. Cl. C07D 403/12; A61K 31/501; A61K 31/506; C07D 401/12; C07D 401/14; C07D 413/12; C07D 417/12; C07D 417/14

(52) U.S. Cl. CPC C07D 403/12; A61K 31/501; A61K 31/506; C07D 401/12; C07D 401/14; C07D 413/12; C07D 417/12; C07D 417/14

(57) ABSTRACT

There are disclosed compounds of the following formula (I) or a stereoisomer or pharmaceutically acceptable salt thereof, wherein all substituents are as defined herein, which are useful in the modulation of IL-12, IL-23 and/or IFNα, by acting on Tyk-2 to cause signal transduction inhibition. The compounds of the invention may be useful for treating neurodegenerative diseases or disorders.

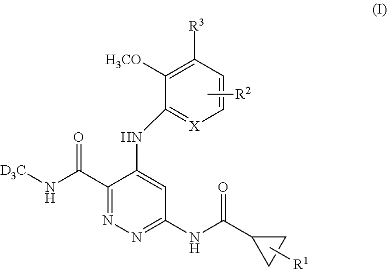

### CROSS-REFERENCE TO RELATED APPLICATIONS

This application claims the benefit of U.S. Provisional Application No. 63/188,498, filed May 14, 2021, and U.S. Provisional Application No. 63/328,835, filed Apr. 8, 2022, the disclosures of which are incorporated herein by reference in its entirety.

### FIELD OF THE INVENTION

This invention relates to compounds useful in the modulation of IL-12, IL-23 and/or IFNα by acting on Tyk-2 to cause signal transduction inhibition. Provided herein are -substituted heterocyclic compounds, compositions comprising such compounds, and methods of their use. The invention further pertains to pharmaceutical compositions containing at least one compound according to the invention that are useful for the treatment of conditions related to the modulation of IL-12, IL-23 and/or IFNα in a mammal. In particular, this invention relates to compounds which show utility against neurodegenerative diseases.

### BACKGROUND OF THE INVENTION

The heterodimeric cytokines interleukin (IL)-12 and IL-23, which share a common p40 subunit, are produced by activated antigen-presenting cells and are critical in the differentiation and proliferation of Th1 and Th17 cells, two effector T cell lineages which play key roles in autoimmunity. IL-23 is composed of the p40 subunit along with a unique p19 subunit. IL-23, acting through a heterodimeric receptor composed of IL-23R and IL-12Rβ1, is essential for the survival and expansion of Th17 cells which produce pro-inflammatory cytokines such as IL-17A, IL-17F, IL-6 and TNF-α (McGeachy, M. J. et al., “The link between IL-23 and Th17 cell-mediated immune pathologies”, Semin. Immunol., 19:372-376 (2007)). These cytokines are critical in mediating the pathobiology of a number of autoimmune diseases, including rheumatoid arthritis, multiple sclerosis, inflammatory bowel disease, and lupus. IL-12, in addition to the p40 subunit in common with IL-23, contains a p35 subunit and acts through a heterodimeric receptor composed of IL-12Rβ1 and IL-12Rβ2. IL-12 is essential for Th1 cell development and secretion of IFNγ, a cytokine which plays a critical role in immunity by stimulating MHC expression, class switching of B cells to IgG subclasses, and the activation of macrophages (Gracie, J. A. et al., “Interleukin-12 induces interferon-gamma-dependent switching of IgG alloantibody subclass”, Eur. J. Immunol., 26:1217-1221 (1996); Schroder, K. et al., “Interferon-gamma: an overview of signals, mechanisms and functions”, J. Leukoc. Biol., 75(2):163-189 (2004)).

The importance of the p40-containing cytokines in autoimmunity is demonstrated by the discovery that mice deficient in either p40, p19, or IL-23R are protected from disease in models of multiple sclerosis, rheumatoid arthritis, inflammatory bowel disease, lupus and psoriasis, among others (Kyttaris, V. C. et al., “Cutting edge: IL-23 receptor deficiency prevents the development of lupus nephritis in C57BL/6-lpr/lpr mice”, J. Immunol., 184:4605-4609 (2010); Hong, K. et al., “IL-12, independently of IFN-gamma, plays a crucial role in the pathogenesis of a murine psoriasis like skin disorder”, J. Immunol., 162:7480-7491 (1999); Hue, S. et al., “Interleukin-23 drives innate and T cell-mediated intestinal inflammation”, J. Exp. Med., 203:2473-2483 (2006); Cua, D. J. et al., “Interleukin-23 rather than interleukin-12 is the critical cytokine for autoimmune inflammation of the brain”, Nature, 421:744-748 (2003); Murphy, C. A. et al., “Divergent pro- and anti-inflammatory roles for IL-23 and IL-12 in joint autoimmune inflammation”, J. Exp. Med., 198:1951-1957 (2003)).

In human disease, high expression of p40 and p19 has been measured in psoriatic lesions, and Th17 cells have been identified in active lesions in the brain from MS patients and in the gut mucosa of patients with active Crohn's disease (Lee, E. et al., “Increased expression of interleukin 23 p19 and p40 in lesional skin of patients with psoriasis vulgaris”, J. Exp. Med., 199:125-130 (2004); Tzartos, J. S. et al., “Interleukin-17 production in central nervous system infiltrating T cells and glial cells is associated with active disease in multiple sclerosis”, Am. J. Pathol., 172:146-155 (2008)). The mRNA levels of p19, p40, and p35 in active SLE patients were also shown to be significantly higher compared with those in inactive SLE patients (Huang, X. et al., “Dysregulated expression of interleukin-23 and interleukin-12 subunits in systemic lupus erythematosus patients”, Mod. Rheumatol., 17:220-223 (2007)), and T cells from lupus patients have a predominant Th1 phenotype (Tucci, M. et al., “Overexpression of interleukin-12 and T helper 1 predominance in lupus nephritis”, Clin. Exp. Immunol., 154:247-254 (2008)).

Moreover, genome-wide association studies have identified a number of loci associated with chronic inflammatory and autoimmune diseases that encode factors that function in the IL-23 and IL-12 pathways. These genes include IL23A, IL12A, IL12B, IL12RB1, IL12RB2, IL23R, JAK2, TYK2, STAT3, and STAT4 (Lees, C. W. et al., “New IBD genetics: common pathways with other diseases”, Gut, 60:1739-1753 (2011); Tao, J. H. et al., “Meta-analysis of TYK2 gene polymorphisms association with susceptibility to autoimmune and inflammatory diseases”, Mol. Biol. Rep., 38:4663-4672 (2011); Cho, J. H. et al., “Recent insights into the genetics of inflammatory bowel disease”, Gastroenterology, 140:1704-1712 (2011)).

Indeed, anti-p40 treatment, which inhibits both IL-12 and IL-23, as well as IL-23-specific anti-p19 therapies have been shown to be efficacious in the treatment of autoimmunity in diseases including psoriasis, Crohn's Disease and psoriatic arthritis (Leonardi, C. L. et al., “PHOENIX 1 study investigators. Efficacy and safety of ustekinumab, a human interleukin-12/23 monoclonal antibody, in patients with psoriasis: 76-week results from a randomized, double-blind, placebo-controlled trial (PHOENIX 1)”, Lancet, 371:1665-1674 (2008); Sandborn, W. J. et al., “Ustekinumab Crohn's Disease Study Group. A randomized trial of Ustekinumab, a human interleukin-12/23 monoclonal antibody, in patients with moderate-to-severe Crohn's disease”, Gastroenterology, 135:1130-1141 (2008); Gottlieb, A. et al., “Ustekinumab, a human interleukin 12/23 monoclonal antibody, for psoriatic arthritis: randomized, double-blind, placebo-controlled, crossover trial”, Lancet, 373:633-640 (2009)). Therefore, agents which inhibit the action of IL-12 and IL-23 may be expected to have therapeutic benefit in human autoimmune disorders.

The Type I group of interferons (IFNs), which include the IFNα members as well as IFNβ, IFNε, IFNκ and IFNω, act through a heterodimer IFNα/β receptor (IFNAR). Type I IFNs have multiple effects in both the innate and adaptive immune systems including activation of both the cellular and humoral immune responses as well as enhancing the expression and release of autoantigens (Hall, J. C. et al., “Type I interferons: crucial participants in disease amplification in autoimmunity”, Nat. Rev. Rheumatol., 6:40-49 (2010)).

In patients with systemic lupus erythematosus (SLE), a potentially fatal autoimmune disease, increased serum levels of interferon (IFN)α (a type I interferon) or increased expression of type I IFN-regulated genes (a so-called IFNα signature) in peripheral blood mononuclear cells and in affected organs has been demonstrated in a majority of patients (Bennett, L. et al., “Interferon and granulopoiesis signatures in systemic lupus erythematosus blood”, J. Exp. Med., 197:711-723 (2003); Peterson, K. S. et al., “Characterization of heterogeneity in the molecular pathogenesis of lupus nephritis from transcriptional profiles of laser-captured glomeruli”, J. Clin. Invest., 113:1722-1733 (2004)), and several studies have shown that serum IFNα levels correlate with both disease activity and severity (Bengtsson, A. A. et al., “Activation of type I interferon system in systemic lupus erythematosus correlates with disease activity but not with antiretroviral antibodies”, Lupus, 9:664-671 (2000)). A direct role for IFNα in the pathobiology of lupus is evidenced by the observation that the administration of IFNα to patients with malignant or viral diseases can induce a lupus-like syndrome. Moreover, the deletion of the IFNAR in lupus-prone mice provides high protection from autoimmunity, disease severity and mortality (Santiago-Raber, M. L. et al., “Type-I interferon receptor deficiency reduces lupus-like disease in NZB mice”, J. Exp. Med., 197:777-788 (2003)), and genome-wide association studies have identified loci associated with lupus that encode factors that function in the type I interferon pathway, including IRF5, IKBKE, TYK2, and STAT4 (Deng, Y. et al., “Genetic susceptibility to systemic lupus erythematosus in the genomic era”, Nat. Rev. Rheumatol., 6:683-692 (2010); Sandling, J. K. et al., “A candidate gene study of the type I interferon pathway implicates IKBKE and TL8 as risk loci for SLE”, Eur. J. Hum. Genet., 19:479-484 (2011)). In addition to lupus, there is evidence that aberrant activation of type I interferon-mediated pathways are important in the pathobiology of other autoimmune diseases such as Sjögren's syndrome and scleroderma (Båve, U. et al., “Activation of the type I interferon system in primary Sjögren's syndrome: a possible etiopathogenic mechanism”, Arthritis Rheum., 52:1185-1195 (2005); Kim, D. et al., “Induction of interferon-alpha by scleroderma sera containing autoantibodies to topoisomerase I: association of higher interferon-alpha activity with lung fibrosis”, Arthritis Rheum., 58:2163-2173 (2008)). Therefore, agents which inhibit the action of type I interferon responses may be expected to have therapeutic benefit in human autoimmune disorders.

Tyrosine kinase 2 (Tyk2) is a member of the Janus kinase (JAK) family of nonreceptor tyrosine kinases and has been shown to be critical in regulating the signal transduction cascade downstream of receptors for IL-12, IL-23 and type I interferons in both mice (Ishizaki, M. et al., “Involvement of Tyrosine Kinase-2 in Both the IL-12/Th1 and IL-23/Th17 Axes In vivo”, J. Immunol., 187:181-189 (2011); Prchal-Murphy, M. et al., “TYK2 kinase activity is required for functional type I interferon responses in vivo”, PLoS One, 7:e39141 (2012)) and humans (Minegishi, Y. et al., “Human tyrosine kinase 2 deficiency reveals its requisite roles in multiple cytokine signals involved in innate and acquired immunity”, Immunity, 25:745-755 (2006)). Tyk2 mediates the receptor-induced phosphorylation of members of the STAT family of transcription factors, an essential signal that leads to the dimerization of STAT proteins and the transcription of STAT-dependent pro-inflammatory genes. Tyk2-deficient mice are resistant to experimental models of colitis, psoriasis and multiple sclerosis, demonstrating the importance of Tyk2-mediated signaling in autoimmunity and related disorders (Ishizaki, M. et al., “Involvement of Tyrosine Kinase-2 in Both the IL-12/Th1 and IL-23/Th17 Axes In vivo”, J. Immunol., 187:181-189 (2011); Oyamada, A. et al., “Tyrosine kinase 2 plays critical roles in the pathogenic CD4 T cell responses for the development of experimental autoimmune encephalomyelitis”, J. Immunol., 183:7539-7546 (2009)).

In humans, individuals expressing an inactive variant of Tyk2 are protected from multiple sclerosis and possibly other autoimmune disorders (Couturier, N. et al., “Tyrosine kinase 2 variant influences T lymphocyte polarization and multiple sclerosis susceptibility”, Brain, 134:693-703 (2011)). Genome-wide association studies have shown other variants of Tyk2 to be associated with autoimmune disorders such as Crohn's Disease, psoriasis, systemic lupus erythematosus, and rheumatoid arthritis, further demonstrating the importance of Tyk2 in autoimmunity (Ellinghaus, D. et al., “Combined Analysis of Genome-wide Association Studies for Crohn Disease and Psoriasis Identifies Seven Shared Susceptibility Loci”, Am. J. Hum. Genet., 90:636-647 (2012); Graham, D. et al., “Association of polymorphisms across the tyrosine kinase gene, TYK2 in UK SLE families”, Rheumatology (Oxford), 46:927-930 (2007); Eyre, S. et al., “High-density genetic mapping identifies new susceptibility loci for rheumatoid arthritis”, Nat. Genet., 44:1336-1340 (2012)).

In view of the conditions that may benefit by treatment involving the modulation of cytokines and/or interferons, new compounds capable of modulating cytokines and/or interferons, such as IL-12, IL-23 and/or IFNα, and methods of using these compounds may provide substantial therapeutic benefits to a wide variety of patients in need thereof.

### SUMMARY OF THE INVENTION

The invention is directed to compounds of Formula I, infra, that which are useful as modulators of IL-12, IL-23 and/or IFNα by inhibiting Tyk2-mediated signal transduction.

The present invention also provides processes and intermediates for making the compounds of the present invention.

The present invention also provides pharmaceutical compositions comprising a pharmaceutically acceptable carrier and at least one of the compounds of the present invention.

The present invention also provides a method for the modulation of IL-12, IL-23 and/or IFNα by inhibiting Tyk-2-mediated signal transduction comprising administering to a host in need of such treatment a therapeutically effective amount of at least one of the compounds of the present invention.

The present invention also provides a method for treating neurodegenerative diseases, comprising administering to a host in need of such treatment a therapeutically effective amount of at least one of the compounds of the present invention.

The present invention also provides the compounds of the present invention for use in therapy.

These and other features of the invention will be set forth in the expanded form as the disclosure continues.

### DETAILED DESCRIPTION OF THE EMBODIMENTS OF THE INVENTION

In a first aspect of the present invention, there is provided a compound of formula

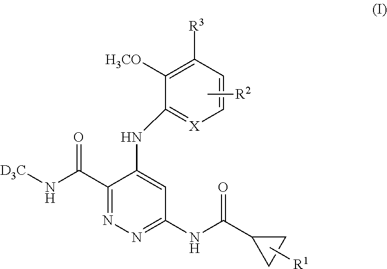

or a stereoisomer or pharmaceutically acceptable salt thereof,

wherein

X is —N— or —CH—;

R¹ is C₁₋₃ alkyl;

R² is F or C₁₋₃ alkyl;

R³ is oxadiazol, oxazol, pyridine, pyrimidine, thiadiazol, pyrazine, thiazol, pyrazol or isothiazole, all of which are substituted with 0-3 R^{3a} groups;

R^{3a} is H, F, Cl, CN, NO₂, C₁₋₄ alkyl, O—C₁₋₄ alkyl, C₃₋₆ cycloalkyl, (CH₂)ₙF, CHF₂; hydroxy C₁₋₃ alkyl or cyano C₁₋₃ alkyl; and

n is 1 or 2.

In a second aspect of the invention, there is provided the compound of formula II

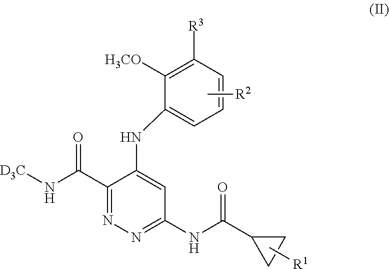

or a stereoisomer or pharmaceutically acceptable salt thereof,

wherein

R¹ is C₁₋₃ alkyl;

R² is F or C₁₋₃ alkyl;

R³ is oxadiazol, oxazol, pyridine, pyrimidine, thiadiazol, pyrazine, thiazol, pyrazol or isothiazole, all of which are substituted with 0-3 R^{3a} groups;

R^{3a} is H, F, Cl, CN, NO₂, C₁₋₄ alkyl, O—C₁₋₄ alkyl, C₃₋₆ cycloalkyl, (CH₂)ₙF, CHF₂; hydroxy C₁₋₃ alkyl or cyano C₁₋₃ alkyl; and

n is 1 or 2.

In a third aspect of the invention, there is provided the compound of formula III

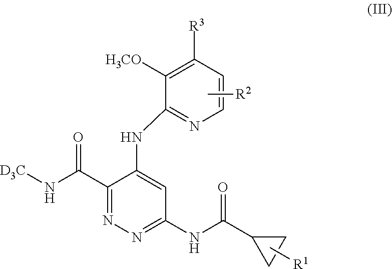

or a stereoisomer or pharmaceutically acceptable salt thereof,

wherein

R¹ is C₁₋₃ alkyl;

R² is F or C₁₋₃ alkyl;

R³ is oxadiazol, oxazol, pyridine, pyrimidine, thiadiazol, pyrazine, thiazol, pyrazol or isothiazole, all of which are substituted with 0-3 R^{3a} groups;

R^{3a} is H, F, Cl, CN, NO₂, C₁₋₄ alkyl, O—C₁₋₄ alkyl, C₃₋₆ cycloalkyl, (CH₂)ₙF, CHF₂; hydroxy C₁₋₃ alkyl or cyano C₁₋₃ alkyl; and

n is 1 or 2.

In another aspect, there is provided a compound selected from the exemplified examples within the scope of the first aspect, or a pharmaceutically acceptable salt thereof.

In another aspect, there is provided a compound selected from any subset list of compounds within the scope of any of the above aspects.

In another aspect, there is provided a compound (IUPAC naming convention) or a pharmaceutically acceptable salt thereof, selected from
6-cyclopropaneamido-4-({3-[5-(fluoromethyl)-1,2,4-oxadiazol-3-yl]-2-methoxyphenyl}amino)-N-(²H3)methylpyridazine-3-carboxamide,
6-cyclopropaneamido-4-[(2-methoxy-3-{5-[(propan-2-yloxy)methyl]-1,2,4-oxadiazol-3-yl}phenyl)amino]-N-(²H3)methylpyridazine-3-carboxamide,
6-cyclopropaneamido-4-({3-[5-(ethoxymethyl)-1,2,4-oxadiazol-3-yl]-2-methoxyphenyl}amino)-N-(²H3)methylpyridazine-3-carboxamide,
6-cyclopropaneamido-4-({2-methoxy-3-[5-(2-methoxyethyl)-1,2,4-oxadiazol-3-yl]phenyl}amino)-N-(²H3)methylpyridazine-3-carboxamide,
6-cyclopropaneamido-4-({3-[5-(2-fluoroethyl)-1,2,4-oxadiazol-3-yl]-2-methoxyphenyl}amino)-N-(²H3)methylpyridazine-3-carboxamide,
6-cyclopropaneamido-4-{[2-methoxy-3-(1,3-oxazol-4-yl)phenyl]amino}-N-(²H3)methylpyridazine-3-carboxamide,
6-cyclopropaneamido-4-({3-[5-(difluoromethyl)pyridin-2-yl]-2-methoxyphenyl}amino)-N-(²H3)methylpyridazine-3-carboxamide,
6-cyclopropaneamido-4-{[2-methoxy-3-(6-methylpyrimidin-4-yl)phenyl]amino}-N-(²H3)methylpyridazine-3-carboxamide,
6-cyclopropaneamido-4-{[2-methoxy-3-(1,2,4-thiadiazol-5-yl)phenyl]amino}-N-(²H3)methylpyridazine-3-carboxamide,
6-cyclopropaneamido-4-{[3-(5-fluoropyrazin-2-yl)-2-methoxyphenyl]amino}-N-(²H3)methylpyridazine-3-carboxamide,
6-cyclopropaneamido-4-{[3-(5-cyclopropylpyrazin-2-yl)-2-methoxyphenyl]amino}-N-(²H3)methylpyridazine-3-carboxamide,
6-cyclopropaneamido-4-{[2-methoxy-3-(5-nitropyrazin-2-yl)phenyl]amino}-N-(²H3)methylpyridazine-3-carboxamide,
6-cyclopropaneamido-4-{[2-methoxy-3-(3-methyl-1,2,4-thiadiazol-5-yl)phenyl]amino}-N-(²H3)methylpyridazine-3-carboxamide,
6-cyclopropaneamido-4-({3-[5-(1-hydroxycyclobutyl)pyrazin-2-yl]-2-methoxy-5-methylphenyl}amino)-N-(²H3)methylpyridazine-3-carboxamide,
6-cyclopropaneamido-4-({5-fluoro-3-[5-(1-hydroxycyclobutyl)pyrazin-2-yl]-2-methoxyphenyl}amino)-N-(²H3)methylpyridazine-3-carboxamide,
6-cyclopropaneamido-4-{[3-(5-cyclopropylpyrazin-2-yl)-5-fluoro-2-methoxyphenyl]amino}-N-(²H3)methylpyridazine-3-carboxamide,
6-cyclopropaneamido-4-{[2-methoxy-3-(2-methyl-1,3-oxazol-4-yl)phenyl]amino}-N-(²H3)methylpyridazine-3-carboxamide,
6-cyclopropaneamido-4-{[3-(2,5-dimethyl-1,3-oxazol-4-yl)-2-methoxyphenyl]amino}-N-(²H3)methylpyridazine-3-carboxamide,
6-cyclopropaneamido-4-({3-[5-(difluoromethyl)-1,2,4-oxadiazol-3-yl]-2-methoxyphenyl}amino)-N-(²H3)methylpyridazine-3-carboxamide,
6-cyclopropaneamido-4-({4-[5-(2-hydroxypropan-2-yl)pyrazin-2-yl]-3-methoxypyridin-2-yl}amino)-N-(²H3)methylpyridazine-3-carboxamide,
6-cyclopropaneamido-4-{[2-methoxy-3-(2-methyl-1,3-thiazol-4-yl)phenyl]amino}-N-(²H3)methylpyridazine-3-carboxamide,
6-cyclopropaneamido-4-{[2-methoxy-3-(5-methoxypyrazin-2-yl)phenyl]amino}-N-(²H3)methylpyridazine-3-carboxamide,
6-cyclopropaneamido-4-{[2-methoxy-3-(5-methylpyrazin-2-yl)phenyl]amino}-N-(²H3)methylpyridazine-3-carboxamide,
4-({3-[5-(1-cyano-1-methylethyl)pyridin-2-yl]-2-methoxyphenyl}amino)-6-cyclopropaneamido-N-(²H3)methylpyridazine-3-carboxamide,
4-{[2-methoxy-3-(2-methyl-2H-1,2,3,4-tetrazol-5-yl)phenyl]amino}-N-(²H3)methyl-6-[(1R,2R)-2-methylcyclopropaneamido]pyridazine-3-carboxamide,
6-cyclopropaneamido-4-{[4-fluoro-2-methoxy-3-(1-methyl-1H-pyrazol-4-yl)phenyl]amino}-N-(²H3)methylpyridazine-3-carboxamide,
6-cyclopropaneamido-4-{[5-fluoro-2-methoxy-3-(1-methyl-1H-pyrazol-4-yl)phenyl]amino}-N-(²H3)methylpyridazine-3-carboxamide,
6-cyclopropaneamido-4-{[3-(1-cyclopropyl-1H-pyrazol-4-yl)-2-methoxyphenyl]amino}-N-(²H3)methylpyridazine-3-carboxamide,
4-{[3-(5-cyanopyrazin-2-yl)-2-methoxyphenyl]amino}-6-cyclopropaneamido-N-(²H3)methylpyridazine-3-carboxamide,
6-cyclopropaneamido-4-{[4-(1-cyclopropyl-1H-pyrazol-4-yl)-3-methoxypyridin-2-yl]amino}-N-(²H3)methylpyridazine-3-carboxamide,
6-cyclopropaneamido-4-{[4-(1,3-dimethyl-1H-pyrazol-4-yl)-3-methoxypyridin-2-yl]amino}-N-(²H3)methylpyridazine-3-carboxamide,
6-cyclopropaneamido-4-{[4-(1,5-dimethyl-1H-pyrazol-4-yl)-3-methoxypyridin-2-yl]amino}-N-(²H3)methylpyridazine-3-carboxamide,
6-cyclopropaneamido-4-{[3-methoxy-4-(1,3-thiazol-2-yl)pyridin-2-yl]amino}-N-(²H3)methylpyridazine-3-carboxamide,
6-cyclopropaneamido-4-{[2-methoxy-3-(1,2-thiazol-5-yl)phenyl]amino}-N-(²H3)methylpyridazine-3-carboxamide,
6-cyclopropaneamido-4-{[2-methoxy-3-(1,2-thiazol-3-yl)phenyl]amino}-N-(²H3)methylpyridazine-3-carboxamide,
6-cyclopropaneamido-4-{[2-methoxy-3-(1,2-thiazol-4-yl)phenyl]amino}-N-(²H3)methylpyridazine-3-carboxamide,
6-cyclopropaneamido-4-{[5-fluoro-2-methoxy-3-(1,3-oxazol-4-yl)phenyl]amino}-N-(²H3)methylpyridazine-3-carboxamide,
6-cyclopropaneamido-4-{[3-methoxy-4-(1,2-thiazol-4-yl)pyridin-2-yl]amino}-N-(²H3)methylpyridazine-3-carboxamide,
6-cyclopropaneamido-4-{[3-methoxy-4-(3-methyl-1,2,4-thiadiazol-5-yl)pyridin-2-yl]amino}-N-(²H3)methylpyridazine-3-carboxamide,
4-{[3-methoxy-4-(3-methyl-1,2,4-thiadiazol-5-yl)pyridin-2-yl]amino}-N-(²H3)methyl-6-[(1R,2R)-2-methylcyclopropaneamido]pyridazine-3-carboxamide,
6-cyclopropaneamido-4-{[3-methoxy-4-(1,2,4-thiadiazol-5-yl)pyridin-2-yl]amino}-N-(²H3)methylpyridazine-3-carboxamide,
6-cyclopropaneamido-4-({4-[1-(difluoromethyl)-1H-pyrazol-4-yl]-3-methoxypyridin-2-yl}amino)-N-(²H3)methylpyridazine-3-carboxamide, and
6-cyclopropaneamido-4-{[3-(5-ethyl-1,2,4-oxadiazol-3-yl)-2-methoxyphenyl]amino}-N-(²H3)methylpyridazine-3-carboxamide.

In another embodiment, there is provided a pharmaceutical composition comprising one or more compounds of formula I and a pharmaceutically acceptable carrier or diluent.

The present invention is also directed to pharmaceutical compositions useful in treating diseases associated with the modulation of IL-12, IL-23 and/or IFNα by acting on Tyk-2 to cause signal transduction inhibition, comprising compounds of formula I, or pharmaceutically-acceptable salts thereof, and pharmaceutically-acceptable carriers or diluents.

The invention further relates to methods of treating diseases associated with the modulation of IL-12, IL-23, and/or IFNα, comprising administering to a patient in need of such treatment a therapeutically-effective amount of a compound according to formula I.

The present invention also provides processes and intermediates for making the compounds of the present invention.

The present invention also provides a method for treating proliferative, metabolic, allergic, autoimmune and inflammatory diseases (or use of the compounds of the present invention for the manufacture of a medicament for the treatment of these diseases), comprising administering to a host in need of such treatment a therapeutically effective amount of at least one of the compounds of the present invention.

The present invention also provides a method of treating an inflammatory or autoimmune disease (or use of the compounds of the present invention for the manufacture of a medicament for the treatment of these diseases) comprising administering to a patient in need of such treatment a therapeutically-effective amount of a compound of Formula I.

The present invention also provides a method for treating a disease (or use of the compounds of the present invention for the manufacture of a medicament for the treatment of these diseases), comprising administering to a patient in need of such treatment a therapeutically-effective amount of a compound of Formula I, wherein the disease is rheumatoid arthritis, multiple sclerosis, systemic lupus erythematosus (SLE), lupus nephritis, cutaneous lupus, inflammatory bowel disease, psoriasis, Crohn's Disease, psoriatic arthritis, Sjögren's syndrome, systemic scleroderma, ulcerative colitis, Graves' disease, discoid lupus erythematosus, adult onset Stills, systemic onset juvenile idiopathic arthritis, gout, gouty arthritis, type 1 diabetes, insulin dependent diabetes mellitus, sepsis, septic shock, Shigellosis, pancreatitis (acute or chronic), glomerulonephritis, autoimmune gastritis, diabetes, autoimmune hemolytic anemia, autoimmune neutropenia, thrombocytopenia, atopic dermatitis, myasthenia gravis, pancreatitis (acute or chronic), ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease, and chronic demyelinating polyneuropathy.

The present invention also provides a method of treating neurodegenerative disease (or use of the compounds of the present invention for the manufacture of a medicament for the treatment of said diseases), comprising administering to a patient in need of such treatment a therapeutically-effective amount of a compound of Formula I, wherein the disease is selected from as Alzheimer's disease, Parkinson's disease, ALS, Multiple Sclerosis (RMS and/or progressive MS, including CIS, optic neuritis, neuromyelitis optica).

The present invention also provides a method for treating a rheumatoid arthritis (or use of the compounds of the present invention for the manufacture of a medicament for the treatment of rheumatoid arthritis, comprising administering to a patient in need of such treatment a therapeutically-effective amount of a compound of Formula I.

In addition, the present invention also provides a method of treating a condition (or use of the compounds of the present invention for the manufacture of a medicament for the treatment of these conditions) comprising administering to a patient in need of such treatment a therapeutically-effective amount of a compound of Formula I, wherein the condition is selected from acute myelogenous leukemia, chronic myelogenous leukemia, metastatic melanoma, Kaposi's sarcoma, multiple myeloma, solid tumors, ocular neovasculization, and infantile haemangiomas, B cell lymphoma, systemic lupus erythematosus (SLE), rheumatoid arthritis, psoriatic arthritis, multiple vasculitides, idiopathic thrombocytopenic purpura (ITP), myasthenia gravis, allergic rhinitis, multiple sclerosis (MS), transplant rejection, Type I diabetes, membranous nephritis, inflammatory bowel disease, autoimmune hemolytic anemia, autoimmune thyroiditis, cold and warm agglutinin diseases, Evans syndrome, hemolytic uremic syndrome/thrombotic thrombocytopenic purpura (HUS/TTP), sarcoidosis, Sjögren's syndrome, peripheral neuropathies, pemphigus vulgaris and asthma.

The present invention also provides a method of treating an IL-12, IL-23, and/or IFNα mediated disease (or use of the compounds of the present invention for the manufacture of a medicament for the treatment of these diseases), comprising administering to a patient in need of such treatment a therapeutically-effective amount of a compound of formula I.

The present invention also provides a method of treating an IL-12, IL-23 and/or IFNα mediated disease (or use of the compounds of the present invention for the manufacture of a medicament for the treatment of these diseases), comprising administering to a patient in need of such treatment a therapeutically-effective amount of a compound of formula I, wherein the IL-12, IL-23 and/or IFNα mediated disease is a disease modulated by IL-12, IL-23 and/or IFNα.

The present invention also provides a method of treating diseases, comprising administering to a patient in need of such treatment a therapeutically-effective amount of a compound of formula I in combination with other therapeutic agents.

The present invention also provides the compounds of the present invention for use in therapy.

In another embodiment, compounds of formula I are selected from exemplified compounds or combinations of exemplified compounds or other embodiments herein.

In another embodiment are compounds having an IC₅₀<1000 nM in at least one of the assays described below.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### DETAILED DESCRIPTION OF THE INVENTION

The following are definitions of terms used in this specification and appended claims. The initial definition provided for a group or term herein applies to that group or term throughout the specification and claims, individually or as part of another group, unless otherwise indicated.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis- and trans-geometric isomers of the compounds of the present invention are described and may be isolated as a mixture of isomers or as separated isomeric forms. The present compounds can be isolated in optically active or racemic forms. It is well known in the art how to prepare optically active forms, such as by resolution of racemic forms or by synthesis from optically active starting materials. All chiral, (enantiomeric and diastereomeric) and racemic forms and all geometric isomeric forms of a structure are intended, unless the specific stereochemistry or isomer form is specifically indicated.

When any variable (e.g., R³) occurs more than one time in any constituent or formula for a compound, its definition at each occurrence is independent of its definition at every other occurrence. Thus, for example, if a group is shown to be substituted with 0-2 R³, then said group may optionally be substituted with up to two R³ groups and R³ at each occurrence is selected independently from the definition of R³. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent.

Combinations of substituents and/or variables are permissible only if such combinations result in stable compounds.

In cases wherein there are nitrogen atoms (e.g., amines) on compounds of the present invention, these can be converted to N-oxides by treatment with an oxidizing agent (e.g., MCPBA and/or hydrogen peroxides) to afford other compounds of this invention. Thus, all shown and claimed nitrogen atoms are considered to cover both the shown nitrogen and its N-oxide (N→O) derivative.

In accordance with a convention used in the art,

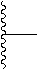

is used in structural formulas herein to depict the bond that is the point of attachment of the moiety or substituent to the core or backbone structure.

A dash “-” that is not between two letters or symbols is used to indicate a point of attachment for a substituent. For example, —CONH₂ is attached through the carbon atom.

The term “optionally substituted” in reference to a particular moiety of the compound of Formula I (e.g., an optionally substituted heteroaryl group) refers to a moiety having 0, 1, 2, or more substituents. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “at least one chemical entity” is interchangeable with the term “a compound”.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

“Alkenyl” or “alkenylene” is intended to include hydrocarbon chains of either straight or branched configuration and having one or more double carbon-carbon bonds that may occur in any stable point along the chain. For example, “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, and the like.

“Alkynyl” or “alkynylene” is intended to include hydrocarbon chains of either straight or branched configuration and having one or more triple carbon-carbon bonds that may occur in any stable point along the chain. For example, “C₂₋₆ alkynyl” (or alkynylene), is intended to include C₂, C₃, C₄, C₅, and C₆ alkynyl groups; such as ethynyl, propynyl, butynyl, pentynyl, hexynyl and the like.

One skilled in the field will understand that, when the designation “CO₂” is used herein, this is intended to refer to the group

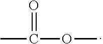

When the term “alkyl” is used together with another group, such as in “arylalkyl”, this conjunction defines with more specificity at least one of the substituents that the substituted alkyl will contain. For example, “arylalkyl” refers to a substituted alkyl group as defined above where at least one of the substituents is an aryl, such as benzyl. Thus, the term aryl(C₀₋₄)alkyl includes a substituted lower alkyl having at least one aryl substituent and also includes an aryl directly bonded to another group, i.e., aryl(Co)alkyl. The term “heteroarylalkyl” refers to a substituted alkyl group as defined above where at least one of the substituents is a heteroaryl.

When reference is made to a substituted alkenyl, alkynyl, alkylene, alkenylene, or alkynylene group, these groups are substituted with one to three substituents as defined above for substituted alkyl groups.

The term “alkoxy” refers to an oxygen atom substituted by alkyl or substituted alkyl, as defined herein. For example, the term “alkoxy” includes the group —O—C₁₋₆alkyl such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. “Lower alkoxy” refers to alkoxy groups having one to four carbons.

It should be understood that the selections for all groups, including for example, alkoxy, thioalkyl, and aminoalkyl, will be made by one skilled in the field to provide stable compounds.

The term “substituted”, as used herein, means that any one or more hydrogens on the designated atom or group is replaced with a selection from the indicated group, provided that the designated atom's normal valence is not exceeded. When a substituent is oxo, or keto, (i.e., ═O) then 2 hydrogens on the atom are replaced. Keto substituents are not present on aromatic moieties. Unless otherwise specified, substituents are named into the core structure. For example, it is to be understood that when (cycloalkyl)alkyl is listed as a possible substituent, the point of attachment of this substituent to the core structure is in the alkyl portion. Ring double bonds, as used herein, are double bonds that are formed between two adjacent ring atoms (e.g., C═C, C═N, or N═N).

Combinations of substituents and/or variables are permissible only if such combinations result in stable compounds or useful synthetic intermediates. A stable compound or stable structure is meant to imply a compound that is sufficiently robust to survive isolation from a reaction mixture to a useful degree of purity, and subsequent formulation into an efficacious therapeutic agent. It is preferred that the presently recited compounds do not contain a N-halo, S(O)₂H, or S(O)H group.

The term “cycloalkyl” refers to cyclized alkyl groups, including mono-, bi- or poly-cyclic ring systems. C₃₋₇ cycloalkyl is intended to include C₃, C₄, C₅, C₆, and C₇ cycloalkyl groups. Example cycloalkyl groups include, but are not limited to, cyclopropyl, cyclobutyl, cyclopentyl, cyclohexyl, norbornyl, and the like. As used herein, “carbocycle” or “carbocyclic residue” is intended to mean any stable 3-, 4-, 5-, 6-, or 7-membered monocyclic or bicyclic or 7-, 8-, 9-, 10-, 11-, 12-, or 13-membered bicyclic or tricyclic ring, any of which may be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, and phenyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a bicyclic ring. When a ring is bridged, the substituents recited for the ring may also be present on the bridge.

The term “aryl” refers to monocyclic or bicyclic aromatic hydrocarbon groups having 6 to 12 carbon atoms in the ring portion, such as phenyl, and naphthyl groups, each of which may be substituted.

Accordingly, in compounds of formula I, the term “cycloalkyl” includes cyclopropyl, cyclobutyl, cyclopentyl, cyclohexyl, cycloheptyl, bicyclooctyl, etc., as well as the following ring systems:

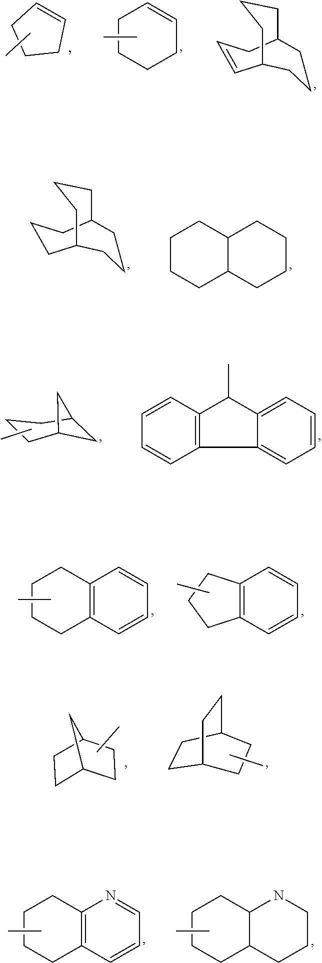

and the like, which optionally may be substituted at any available atoms of the ring(s).

Preferred cycloalkyl groups include cyclopropyl, cyclobutyl, cyclopentyl, and cyclohexyl.

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The terms “heterocycle”, “heterocycloalkyl”, “heterocyclo”, “heterocyclic”, or “heterocyclyl” may be used interchangeably and refer to substituted and unsubstituted 3- to 7-membered monocyclic groups, 7- to 11-membered bicyclic groups, and 10- to 15-membered tricyclic groups, in which at least one of the rings has at least one heteroatom (O, S or N), said heteroatom containing ring preferably having 1, 2, or 3 heteroatoms selected from O, S, and N. Each ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The fused rings completing the bicyclic and tricyclic groups may contain only carbon atoms and may be saturated, partially saturated, or fully unsaturated. The heterocyclo group may be attached at any available nitrogen or carbon atom. As used herein the terms “heterocycle”, “heterocycloalkyl”, “heterocyclo”, “heterocyclic”, and “heterocyclyl” include “heteroaryl” groups, as defined below.

In addition to the heteroaryl groups described below, exemplary monocyclic heterocyclyl groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 1-pyridonyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl. Additional monocyclic heterocyclyl groups include

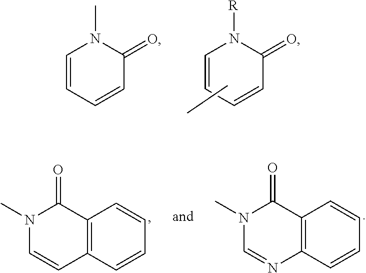

The term “heteroaryl” refers to substituted and unsubstituted aromatic 5- or 6-membered monocyclic groups, 9- or 10-membered bicyclic groups, and 11- to 14-membered tricyclic groups which have at least one heteroatom (O, S or N) in at least one of the rings, said heteroatom-containing ring preferably having 1, 2, or 3 heteroatoms selected from O, S, and N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. As valence allows, if said further ring is cycloalkyl or heterocyclo it is additionally optionally substituted with ═O (oxo).

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, tetrahydroquinolinyl and the like.

Exemplary tricyclic heteroaryl groups include carbazolyl, benzindolyl, phenanthrollinyl, acridinyl, phenanthridinyl, xanthenyl and the like.

In compounds of formula I, preferred heteroaryl groups include:

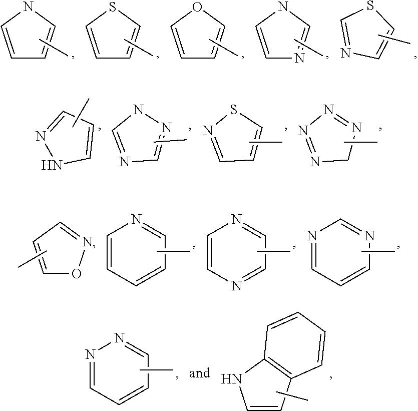

and, and the like, which optionally may be substituted at any available carbon or nitrogen atom.

Unless otherwise indicated, when reference is made to a specifically-named aryl (e.g., phenyl), cycloalkyl (e.g., cyclohexyl), heterocyclo (e.g., pyrrolidinyl, piperidinyl, and morpholinyl) or heteroaryl (e.g., tetrazolyl, imidazolyl, pyrazolyl, triazolyl, thiazolyl, and furyl) the reference is intended to include rings having 0 to 3, preferably 0 to 2, substituents selected from those recited above for the aryl, cycloalkyl, heterocyclo and/or heteroaryl groups, as appropriate.

The term “carbocyclyl” or “carbocyclic” refers to a saturated or unsaturated monocyclic or bicyclic ring in which all atoms of all rings are carbon. Thus, the term includes cycloalkyl and aryl rings. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of mono- and bicyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl and naphthyl. The carbocyclic ring may be substituted in which case the substituents are selected from those recited above for cycloalkyl and aryl groups.

The term “heteroatoms” shall include oxygen, sulfur and nitrogen.

When the term “unsaturated” is used herein to refer to a ring or group, the ring or group may be fully unsaturated or partially unsaturated.

Throughout the specification, groups and substituents thereof may be chosen by one skilled in the field to provide stable moieties and compounds and compounds useful as pharmaceutically acceptable compounds and/or intermediate compounds useful in making pharmaceutically-acceptable compounds.

The compounds of formula I may exist in a free form (with no ionization) or can form salts which are also within the scope of this invention. Unless otherwise indicated, reference to an inventive compound is understood to include reference to the free form and to salts thereof. The term “salt(s)” denotes acidic and/or basic salts formed with inorganic and/or organic acids and bases. In addition, the term “salt(s)” may include zwitterions (inner salts), e.g., when a compound of formula I, contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula I may be formed, for example, by reacting a compound of the formula I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically-acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA (1990), the disclosure of which is hereby incorporated by reference.

All stereoisomers of the compounds of the instant invention are contemplated, either in admixture or in pure or substantially pure form. Stereoisomers may include compounds which are optical isomers through possession of one or more chiral atoms, as well as compounds which are optical isomers by virtue of limited rotation about one or more bonds (atropisomers). The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The present invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

Prodrugs and solvates of the inventive compounds are also contemplated. The term “prodrug” denotes a compound which, upon administration to a subject, undergoes chemical conversion by metabolic or chemical processes to yield a compound of the formula I, and/or a salt and/or solvate thereof. Any compound that will be converted in vivo to provide the bioactive agent (i.e., the compound for formula I) is a prodrug within the scope and spirit of the invention. For example, compounds containing a carboxy group can form physiologically hydrolyzable esters which serve as prodrugs by being hydrolyzed in the body to yield formula I compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula I include C₁₋₆alkylbenzyl, 4-methoxybenzyl, indanyl, phthalyl, methoxymethyl, C₁₋₆alkanoyloxy-C₁₋₆alkyl, e.g., acetoxymethyl, pivaloyloxymethyl or propionyloxymethyl, C₁₋₆alkoxycarbonyloxy-C₁₋₆alkyl, e.g., methoxycarbonyl-oxymethyl or ethoxycarbonyloxymethyl, glycyloxymethyl, phenylglycyloxymethyl, (5-methyl-2-oxo-1,3-dioxolen-4-yl)-methyl and other well known physiologically hydrolyzable esters used, for example, in the penicillin and cephalosporin arts. Such esters may be prepared by conventional techniques known in the art.

Various forms of prodrugs are well known in the art and are described in Rautio, J. et al., Nature Review Drug Discovery, 17, 559-587 (2018).

Compounds of the formula I and salts thereof may exist in their tautomeric form, in which hydrogen atoms are transposed to other parts of the molecules and the chemical bonds between the atoms of the molecules are consequently rearranged. It should be understood that the all tautomeric forms, insofar as they may exist, are included within the invention. Additionally, inventive compounds may have trans- and cis-isomers.

It should further be understood that solvates (e.g., hydrates) of the compounds of Formula I are also with the scope of the present invention. Methods of solvation are generally known in the art.

### UTILITY

The compounds of the invention modulate IL-23-stimulated and IFNα-stimulated cellular functions, including gene transcription. Other types of cellular functions that may be modulated by the compounds of the instant invention include, but are not limited to, IL-12-stimulated responses.

Accordingly, compounds of formula I have utility in treating conditions associated with the modulation of the function of IL-23 and/or IFNα, and particularly the selective inhibition of function of IL-23, IL-12 and/or IFNα, by acting on Tyk2 to mediate signal transduction. Such conditions include IL-23-, IL-12- or IFNα-associated diseases in which pathogenic mechanisms are mediated by these cytokines and the subsequent activation of the Tyk2 pathway with subsequent pro-inflammatory responses which may occur in the peripheral and/or central compartments.

As used herein, the terms “treating” or “treatment” encompass the treatment of a disease state in a mammal, particularly in a human, and include: (a) preventing or delaying the occurrence of the disease state in a mammal, in particular, when such mammal is predisposed to the disease state but has not yet been diagnosed as having it; (b) inhibiting the disease state, i.e., arresting or slowing its development; and/or (c) achieving a full or partial reduction of the symptoms or disease state, and/or alleviating, ameliorating, lessening, or curing the disease or disorder and/or its symptoms.

In view of their activity as modulators of IL-23-, IL-12 and/or IFNα-stimulated cellular responses, compounds of Formula I are useful in treating IL-23-, IL-12- and/or IFNα-associated diseases including, but not limited to, inflammatory diseases such as Crohn's disease, ulcerative colitis, asthma, graft versus host disease, allograft rejection, chronic obstructive pulmonary disease; autoimmune diseases such as Graves' disease, rheumatoid arthritis, systemic lupus erythematosus, cutaneous lupus, lupus nephritis, discoid lupus erythematosus, psoriasis; auto-inflammatory diseases including CAPS, TRAPS, FMF, adult onset stills, systemic onset juvenile idiopathic arthritis, gout, gouty arthritis; metabolic diseases including type 2 diabetes, atherosclerosis, myocardial infarction; destructive bone disorders such as bone resorption disease, osteoarthritis, osteoporosis, multiple myeloma-related bone disorder; proliferative disorders such as acute myelogenous leukemia, chronic myelogenous leukemia; angiogenic disorders such as angiogenic disorders including solid tumors, ocular neovasculization, and infantile haemangiomas; infectious diseases such as sepsis, septic shock, and Shigellosis; neurodegenerative diseases such as Alzheimer's disease, Parkinson's disease, ALS, Multiple Sclerosis (RMS and/or progressive MS, including CIS, optic neuritis, neuromyelitis optica), cerebral ischemias or neurodegenerative disease caused by traumatic injury, oncologic and viral diseases such as metastatic melanoma, Kaposi's sarcoma, multiple myeloma, and HIV infection and CMV retinitis, AIDS, respectively.

More particularly, the specific conditions or diseases that may be treated with the inventive compounds include, without limitation, pancreatitis (acute or chronic), asthma, allergies, adult respiratory distress syndrome, chronic obstructive pulmonary disease, glomerulonephritis, rheumatoid arthritis, systemic lupus erythematosis, cutaneous lupus, lupus nephritis, discoid lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic 3-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, Multiple Sclerosis (RMS and/or progressive MS, including CIS, optic neuritis, neuromyelitis optica), cerebral ischemias or neurodegenerative disease caused by traumatic injury; angiogenic disorders including solid tumors, ocular neovascularization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hypoxia, vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. Preferred methods of treatment are those wherein the condition is selected from Alzheimer's disease, Parkinson's disease, ALS, Multiple Sclerosis (RMS and/or progressive MS, including CIS, optic neuritis, neuromyelitis optica),

When the terms “IL-23-, IL-12- and/or IFNα-associated condition” or “IL-23-, IL-12- and/or IFNα-associated disease or disorder” are used herein, each is intended to encompass all of the conditions identified above as if repeated at length, as well as any other condition that is affected by IL-23, IL-12 and/or IFNα.

The present invention thus provides methods for treating such conditions, comprising administering to a subject in need thereof a therapeutically effective amount of at least one compound of Formula I or a salt thereof. “Therapeutically effective amount” is intended to include an amount of a compound of the present invention that is effective when administered alone or in combination to inhibit IL-23, IL-12 and/or IFNα function and/or treat diseases.

The methods of treating IL-23-, IL-12 and/or IFNα-associated conditions may comprise administering compounds of Formula I alone or in combination with each other and/or other suitable therapeutic agents useful in treating such conditions. Accordingly, “therapeutically effective amount” is also intended to include an amount of the combination of compounds claimed that is effective to inhibit IL-23, IL-12 and/or IFNα function and/or treat diseases associated with IL-23, IL-12 and/or IFNα.

Exemplary of such other therapeutic agents include corticosteroids, rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); non-steroidal anti-inflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus, PROGRAF®); anti-malarials such as hydroxychloroquine; cytotoxic drugs such as azathiprine and cyclophosphamide; TNF-α inhibitors such as tenidap, anti-TNF antibodies or soluble TNF receptor, and rapamycin (sirolimus or RAPAMUNE®) or derivatives thereof.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds. The present invention also provides pharmaceutical compositions capable of treating IL-23-, IL-12- or IFNα-associated conditions by inhibiting Tyk2-mediated signal transduction, including IL-23-, IL-12- and/or IFNα-mediated diseases, as described above.

The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising one or more compounds of Formula I and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include without limitation the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th Edition (1985), which is incorporated herein by reference in its entirety.

The compounds of Formula I may be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of tablets, capsules, granules, powders, or liquid formulations including syrups; topically, such as in the form of solutions, suspensions, gels or ointments; sublingually; bucally; parenterally, such as by subcutaneous, intravenous, intramuscular or intrasternal injection or infusion techniques (e.g., as sterile injectable aq. or non-aq. solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compounds may be administered in a form suitable for immediate release or extended release.

Immediate release or extended release may be achieved with suitable pharmaceutical compositions or, particularly in the case of extended release, with devices such as subcutaneous implants or osmotic pumps.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The therapeutically-effective amount of a compound of the present invention may be determined by one of ordinary skill in the art, and includes exemplary dosage amounts for a mammal of from about 0.05 to 1000 mg/kg; 1-1000 mg/kg; 1-50 mg/kg; 5-250 mg/kg; 250-1000 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species that are affected by modulation of IL-23, IL-12 and/or IFNα-mediated functions.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Third Edition, Wiley and Sons, 1999).

The key intermediates shown in FIG. 1 can be assembled to give compound 1 in a variety of ways known to one skilled in the art of synthetic organic chemistry.

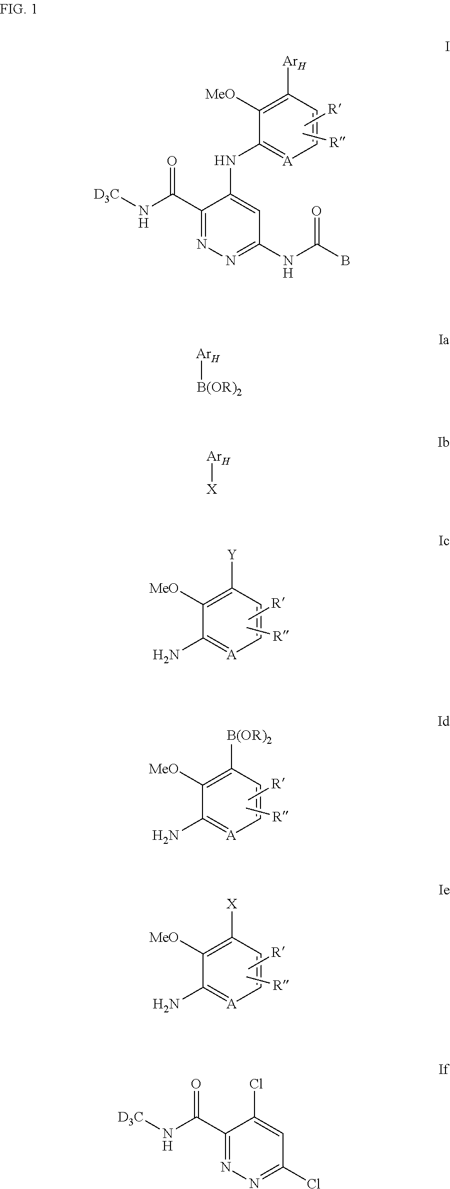

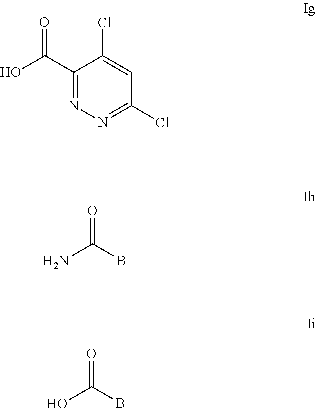

Scheme 1 shows how intermediate Ia and/or Id can be formed from Ib or Ie respectively, in some cases the amine of Ie may need to be protected via standard protecting groups prior to the borylation reaction. The borylation reaction can be carried out via a metal halogen exchange followed by quenching with a trialkylborate, specifically trimethylborate or triisopropylborate. A preferred base for the metal halogen exchange could be isopropylmagnesium chloride-lithium chloride complex in THE at low temperature. Alternatively cross-coupling using a palladium source such as PdCl₂(dppf)[DCM] and a boron source, such as bis(pinacolato)diborane in the presence of potassium acetate, or related, can afford after appropriate time and temperature conversion from the halides to the corresponding boronates.

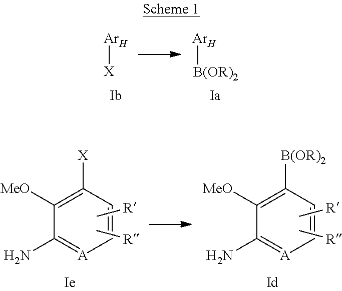

Scheme 2 shows how one skilled in the art can combine intermediate Ia or Ib with intermediate Ie or Id, to provide intermediates of general formula III.) The transformation, commonly referred to as a Suzuki reaction, can be achieved by those skill in the art using transition metal catalyzed coupling of the appropriate boronate with the appropriate halide. More specifically, this transformation can be achieved using a Suzuki type coupling with PdCl₂(dppf)[DCM] as the catalyst and aqueous tribasic potassium phosphate as the base in solvents like 1,4-dioxane at elevated temperatures.

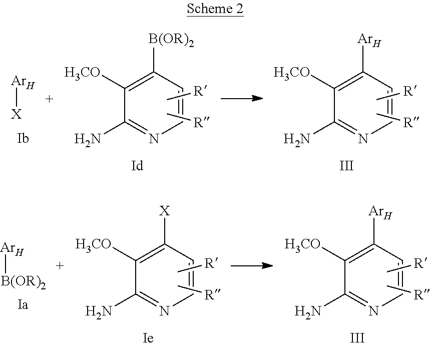

Scheme 3 shows how one skilled in the art of organic synthesis can couple intermediates of general formula If (Moslin, et. al., J. Med. Chem 2019, 62, 8953-8972) with intermediates of general formula III to provide intermediate of general formula IV. The reaction involves mixing the two reagents in an appropriate aprotic solvent, particularly THE or 2-methyl-THF at between 0° C. and 50° C. depending on the particular III and adding the appropriate base, particularly, lithium hexamethyldisilazide, sodium hexamethyldisilazide, potassium hexamethyldisilazide or sodium hydride.

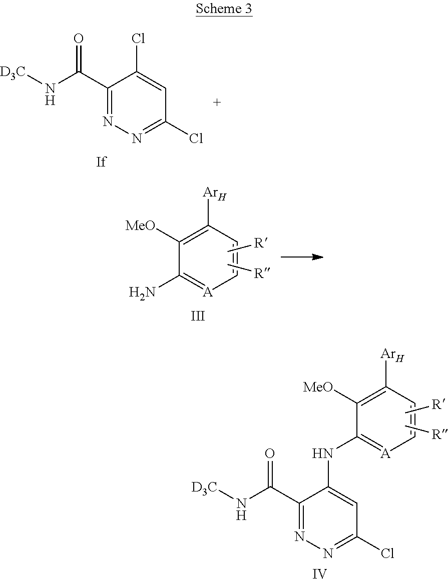

Scheme 4 shows how one skilled in the art of organic synthesis can couple compound IV to the appropriate substrate, in one or one steps, to produce compounds of general formula I. The one step process involves the coupling of compounds of general formula IV with primary amides of general formula Ih under transition metal catalyzed conditions. In particular, favorable conditions for this reaction involve employing a Buchwald type coupling, using Pd₂(dba)₃, as catalyst, xantphos as the ligand and Cs₂CO₃ as the base in 1,4-dioxane as solvent, at elevated temperatures. This catalyst/ligand/base system can be altered in ways known to those skilled in the art. Alternatively, compounds of general formula IV can be treated with primary amines, that can give a product that can be deprotected to afford the corresponding primary amines of general formula V, at elevated temperature in an appropriate solvent, particularly 4-methoxybenzylamine. The product of this reaction can be deprotected to afford V with TFA at elevated temperature. Subsequently, compounds of general formula I can be prepared from V by coupling with an appropriate carboxylic acid under amide coupling conditions known by those skilled in the art.

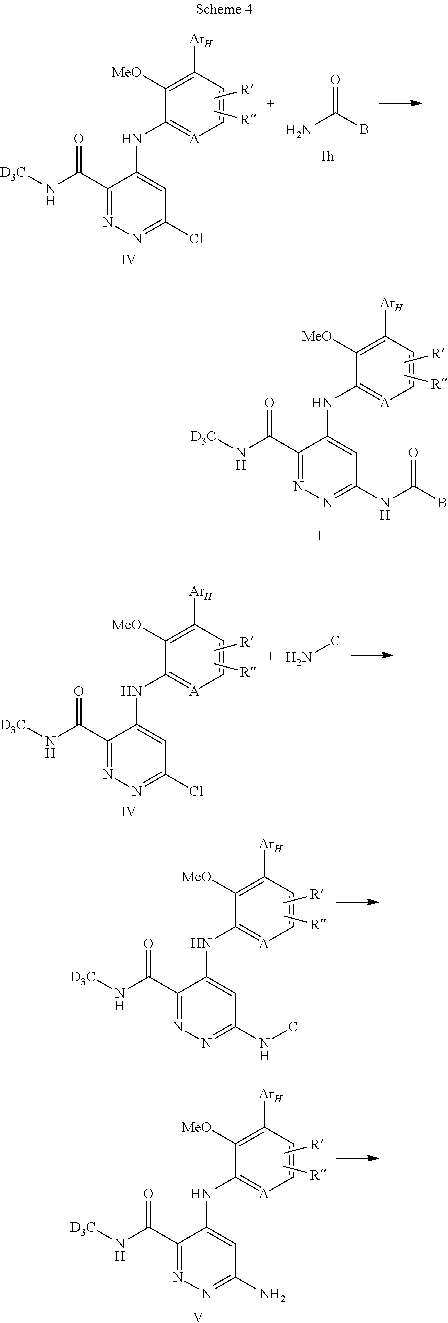

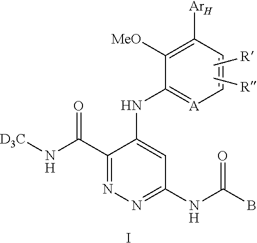

Scheme 5 shows an alternative synthesis of compounds of general formula I. Compound If can be reacted with an aliphatic sulfide nucleophile to generate VI thus enabling the regioselective coupling of amides of type Ih using aforementioned palladium catalysis conditions. Oxidation of the sulfide in VI to the sulfoxide (VII), in this case prior to the palladium catalyzed coupling, reactivates the position to nucleophilic displacement. It is also possible that someone skilled in the art would devise a synthesis where oxidation of the sulfide occurred subsequent to the palladium catalyzed coupling reaction, either route ultimately arrives at VIII. Reaction of VIII with aryl amines of type Ie in the presence of a strong base such as lithium hexamethyldisilazide in a polar aprotic solvent such as THE provides compounds of the general formula IX. Compound IX can then be coupled directly with boronates of type Ia using standard Suzuki conditions to provide I. Alternatively IX can be converted to the boronate X using conditions analogous to the transformation of Ie to Id and then coupled with aryl halides of the general formula Ib.

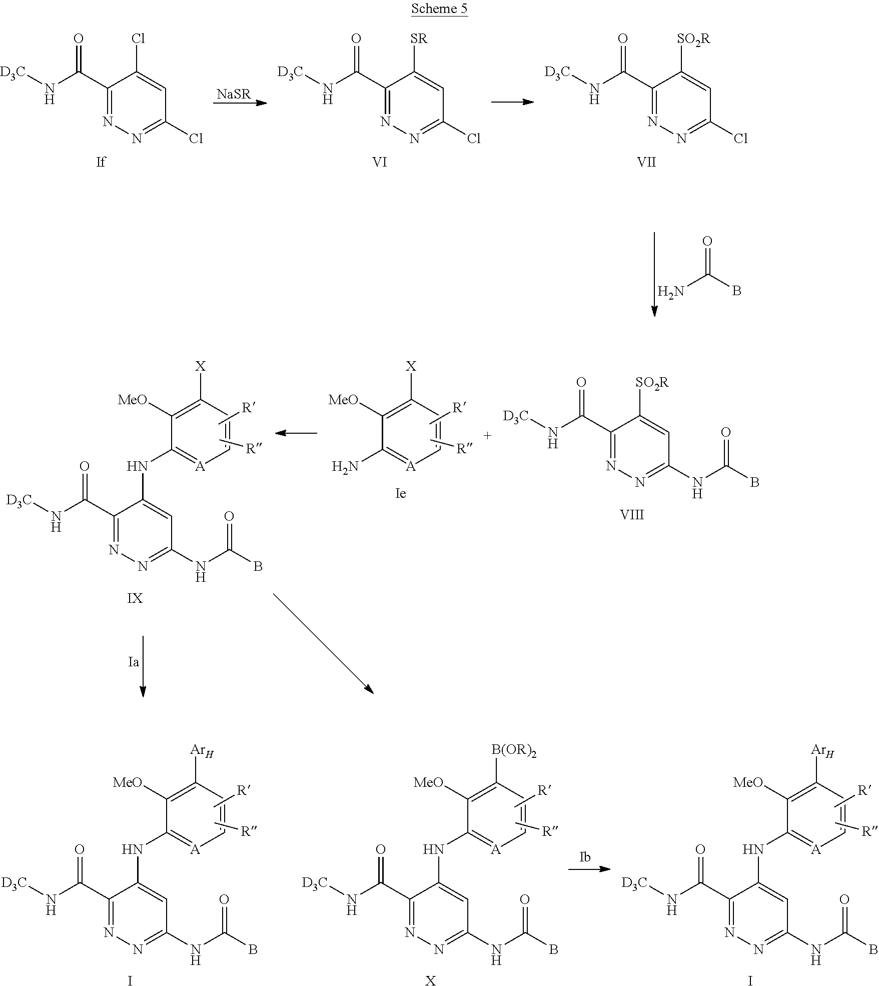

There are many other means to assemble heteroaryl moieties that do not rely upon a palladium-catalyzed reaction, including building them off of functional groups, such as carboxylic acids, amides and cyano groups, already appended to the desired point of attachment. Specific examples are included within this document and for a general description of strategies see Moslin, et. al., U.S. Pat. No. 9,505,748.

### Method A:

Linear gradient of 20% to 100% solvent B over 4 minutes with 0.6-minute hold at 100% B and
followed by 0.1-minute gradient to 20% B and a 0.3-minute hold at 20% B
Solvent A: 5 mm Ammonium formate pH 3.3: ACN (98:02)
Solvent: B: ACN: Buffer (98:02)
Flow Rate: 1.0 ml/min
Column: Kinetex XB-C18 (75×3.0) mm, 2.6 μm
Ultraviolet (“UV”) visualization at 220 nanometers (“nm”).

### Method B:

Linear gradient of 0 to 100% solvent B over 3 minutes, with 0.75 minute hold at 100% B
Ultraviolet (“UV”) visualization at 220 nanometers (“nm”)
Column: Acquity UPLC® BEH (2.1×50 mm) C18 1.7 μM
Flow rate: 1.11 mL/min
Solvent A: 10 mM ammonium acetate, 95% water, 5% acetonitrile
Solvent B: 10 mM ammonium acetate, 5% water, 95% acetonitrile

### Method C:

Linear gradient of 0 to 100% solvent B over 2 minutes (“min”), with 0.5 minute (“min”) hold at 100% B
Ultraviolet (“UV”) visualization at 254 nanometers (“nm”)
Column: Acquity UPLC® BEH C18 1.7 μM
Flow rate: 1 milliliter (“mL”)/min
Solvent A: 0.05% trifluoroacetic acid, 95% water, 5% acetonitrile
Solvent B: 0.05% trifluoroacetic acid, 5% water, 95% acetonitrile

### Method D:

Linear gradient of 2 to 98% solvent B over 1 minute (“min”), with 0.5 minute (“min”) hold at 98% B
Ultraviolet (“UV”) visualization at 254 nanometers (“nm”)
Column: Acquity UPLC® BEH C18 1.7 μM
Flow rate: 0.8 milliliters (“mL”)/min
Solvent A: water
Solvent B: acetonitrile

### Method E:

Linear gradient of 5% to 95% solvent B over 2.5 minutes with 1.5-minute hold at 95% B and followed by 0.5-minute gradient to 5% B and a 1.5-minute hold at 5% B
Solvent A: 0.1% TFA in H₂O
Solvent: B: 0.1% TFA in ACN
Flow Rate: 1.5 ml/min
Column: XBridge C8 (50×4.6) mm, 3.5 μm
Ultraviolet (“UV”) visualization at 220 nanometers (“nm”).

### Method F:

Linear gradient of 20% to 100% solvent B over 4 minutes with 0.6-minute hold at 100% B and followed by 0.1-minute gradient to 20% B and a 0.3-minute hold at 20% B.
Solvent A: 5 mM Ammonium formate pH 3.3: ACN (98:02)
Solvent: B: ACN: Buffer (98:02)
Flow Rate: 1.0 ml/min
Column: Kinetex XB-C18 (75×3.0) mm, 2.6 μm
Ultraviolet (“UV”) visualization at 220 nanometers (“nm”).

### Method G:

Linear gradient of 5% to 95% solvent B over 2.5 minutes with 1.5-minute hold at 95% B and followed by 0.5-minute gradient to 5% B and a 1.5-minute hold at 5% B
Solvent A: 0.1% TFA in H₂O
Solvent: B: 0.1% TFA in ACN
Flow Rate: 1.5 ml/min
Column: XBridge C₈ (50×4.6) mm, 3.5 μm
Ultraviolet (“UV”) visualization at 220 nanometers (“nm”).

### GCMS Method H:

Chromatographic column: HP-5 (30 μm×320 μm×0.25 μm)
Column length 30 m, internal diameter 0.32 mm, thickness 0.25 μm
Inlet temperature: 250° C.; Carrier gas: He
Detector temperature: 300° C.; Column flow 2 mL/min;
Airflow 400 mL/min; H₂ flow 40 mL/min.
Heating schedule: 120° C. hold time 3 min; Then raise to 300° C. with a 40° C./min speed and hold for 2 min.

Source temperature: 230° C.

Recorded on: 7890B GC with 5977B MSD from Agilent technologies.

### Method I:

Linear gradient of 0 to 100% solvent B over 3 minutes (“min”), with 0.5 minute (“min”) hold at 100% B
Ultraviolet (“UV”) visualization at 254 nanometers (“nm”)
Column: Acquity UPLC® BEH C18 1.7 μM
Flow rate: 1 milliliter (“mL”)/min
Solvent A: 0.05% trifluoroacetic acid, 95% water, 5% acetonitrile
Solvent B: 0.05% trifluoroacetic acid, 5% water, 95% acetonitrile

| Abbreviation | Meaning |
|---|---|
| ACN | Acetonitrile |
| DIPEA | Diisopropylamine |
| LiHMDS | Lithium bis(trimethylsilyl)amide |
| EtOH | Ethanol |
| EtOAc | Ethyl acetate |
| THF | Tetrahydrofuran |
| DCM | Dichloromethane |
| TBAF | Tetra-n-butylammonium fluoride |
| DMF | N,N′-Dimethylformamide |
| TFA | Trifluoroacetic acid |
| DAST | Diethylaminosulfur trifluoride |
| Tf₂O | Trifluoromethanesulphonic anhydride |
| dba | dibenzylideneacetone |
| Xantphos | 4,5-Bis(diphenylphosphino)-9,9-dimethylxanthene |
| dcpf | 1,1′-Bis(dicyclohexylphosphino)ferrocene |
| dppf | 1, 1′-Bis(diphenylphosphino)ferrocene |
| MeOH | Methanol |
| DIC | N, N′-Diisopropylcarbodiimide |
| HPLC | high pressure liquid chromatography |
| DIAD | Diisopropyl azodicarboxylate |
| LC | liquid chromatography |
| MS | mass spectrometry |
| rt | Room temperature |
| Pd/C | palladium on carbon |
| Et | Ethyl |
| Me | Methyl |
| h | hours |
| ° C. | °Celsius |
| PBSF | Perfluorobutanesulfonyl fluoride |
| HATU | 1-[Bis(dimethylamino)methylene]-1H-1,2,3- |
| | triazolo[4,5-b]pyridinium 3-oxid |
| | hexafluorophosphate |
| DMA | dimethylacetamide |
| MW | microwave |
| AcOH | Acetic acid |
| DMAP | 4-dimethyl aminopyridine |
| Boc | Tert-butoxy carbonyl |
| AcCl | Acetyl chloride |
| min | minutes |
| MHz | megahertz |
| m-CPBA | meta-Chloroperoxybenzoic acid |

### Intermediate 1

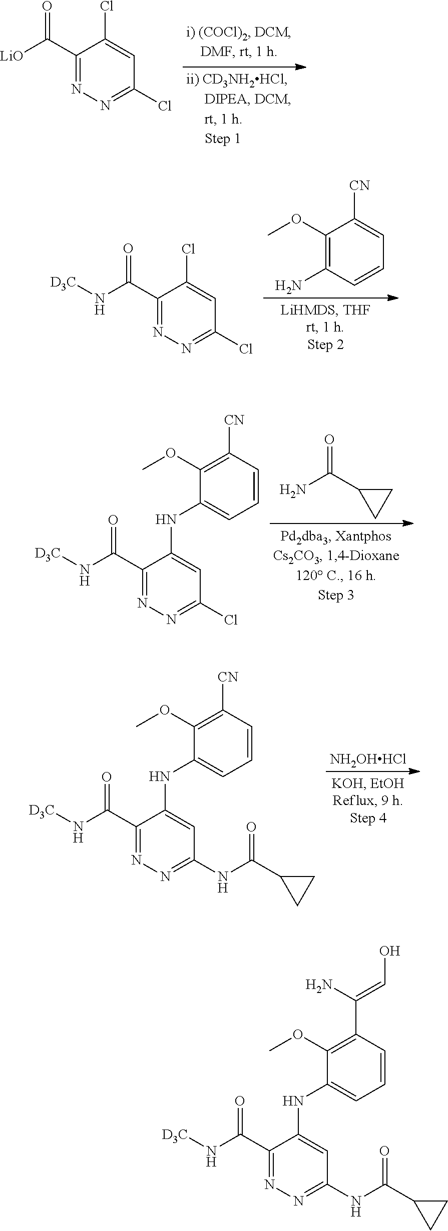

### Step 1

To a solution of lithium 4,6-dichloropyridazine-3-carboxylate (10.0 g, 51.8 mmol) in DCM (200 mL) was added oxalyl chloride (9.07 mL, 104.0 mmol) and 1 mL DMF at 0° C. The reaction mixture was allowed to warm to rt over 1 h. The solvent was removed under reduced pressure and DCM (200 mL) was added to the reaction mixture. The resulting solution was cooled to −30° C., and deuterated methyl amine hydrochloride (4.02 g, 57.0 mmol) was added, followed by DIPEA (18.1 mL, 104.0 mmol). The reaction mixture was stirred at −30° C. for 1 h, then warmed to room temperature and stirring was continued further for 1 h. The reaction mixture was concentrated under reduced pressure and the crude residue was dissolved in ethyl acetate (200 mL) and washed with water (2×100 mL) and brine (100 mL). Organic layer dried over anhydrous Na₂SO₄ and then concentrated under reduced pressure. The crude residue was purified by silica gel column chromatography (10-15% of ethyl acetate in pet ether) to obtain the desired product 4,6-dichloro-N-(methyl-d₃)pyridazine-3-carboxamide (4.1 g, 19.42 mmol, 37.5% yield) as a pale-yellow solid. MS (M+1) m/z: 208.6 (M+H)⁺. LC retention time 1.18 min [G].

### Step 2

LiHMDS (7.65 mL, 7.65 mmol, 1.0 M solution in THF) was added to a solution of 3-amino-2-methoxybenzonitrile (0.35 g, 2.39 mmol) and 4,6-dichloro-N-(methyl-d₃)pyridazine-3-carboxamide (0.5 g, 2.39 mmol) in THE (10 mL) at rt. The reaction mixture was stirred at rt for 1 h, then quenched with saturated aqueous NH₄Cl solution (20 mL). The reaction mixture was partitioned between ethyl acetate (50 mL) and water (40 mL). Organic layer collected, dried over anhydrous Na₂SO₄, concentrated under reduced pressure. The crude product was purified under silica gel column chromatography (30% ethyl acetate in pet ether) to afford 6-chloro-4-((3-cyano-2-methoxyphenyl)amino)-N-(methyl-d₃)pyridazine-3-carboxamide (505 mg, 65.8%) as a brown solid. MS (M+1) m/z: 321.0 (M+H)⁺. LC retention time 2.09 min [F]. ¹H-NMR (400 MHz, DMSO-d₆): δ 11.12 (s, 1H), 9.42 (s, 1H), 7.88 (dd, J=1.60, 8.20 Hz, 1H), 7.68 (dd, J=1.60, 7.80 Hz, 1H), 7.36 (t, J=7.60 Hz, 1H), 7.23 (s, 1H), 3.92 (s, 3H).

### Step 3

To a solution of 6-chloro-4-((3-cyano-2-methoxyphenyl)amino)-N-(methyl-d₃)pyridazine-3-carboxamide (0.5 g, 1.56 mmol), cyclopropanecarboxamide (0.66 g, 7.79 mmol) in dioxane (20 mL) were added Pd₂(dba)₃ (0.14 g, 0.16 mmol), xantphos (0.18 g, 0.31 mmol) and Cs₂CO₃ (1.27 g, 3.90 mmol). The reaction mixture was degassed under N₂ for 5 mins. The vessel was sealed and stirred at 120° C. for 16 h. The reaction mixture was filtered through a celite pad and washed with methanol (50 mL). Collected filtrate was concentrated under reduced pressure and purified via silica gel column chromatography (5% MeOH in DCM) to obtain the desired product 4-((3-cyano-2-methoxyphenyl)amino)-6-(cyclopropanecarboxamido)-N-(methyl-d₃)pyridazine-3-carboxamide (380 mg, 66.0%) as a brown solid. MS (M+1) m/z: 370.0 (M+H)⁺. LC retention time 2.02 min [F].

### Step 4

To a solution of 4-((3-cyano-2-methoxyphenyl)amino)-6-(cyclopropanecarboxamido)-N-(methyl-d₃)pyridazine-3-carboxamide (0.15 g, 0.42 mmol) in ethanol (5 mL) was added hydroxylamine hydrochloride (0.14 g, 2.07 mmol) and KOH (0.11 g, 1.99 mmol). The reaction mixture was heated to reflux at 85° C. for 1 h. An additional aliquot of hydroxylamine hydrochloride (0.14 g, 2.07 mmol) and KOH (0.11 g, 1.99 mmol) were added and the reaction was stirred at 85° C. for 8 h. The reaction mixture was cooled and concentrated under reduced pressure. The crude reaction mixture was partitioned between DCM (50 mL) and water (30 mL). The organic layer was collected and the aqueous layer extracted with DCM (50 mL). The combined organic extracts were dried over anhydrous Na₂SO₄ and concentrated under reduced pressure. The crude residue was purified using silica gel column chromatography (10% MeOH in DCM) to give (Z)-6-(cyclopropanecarboxamido)-4-((3-(N′-hydroxycarbamimidoyl)-2-methoxyphenyl)amino)-N-(methyl-d₃)pyridazine-3-carboxamide (90 mg, 55.1%) as a brown solid. MS (M+1) m/z: 403.0 (M+H)⁺. LC retention time 0.63 min [F].

### Intermediate 2

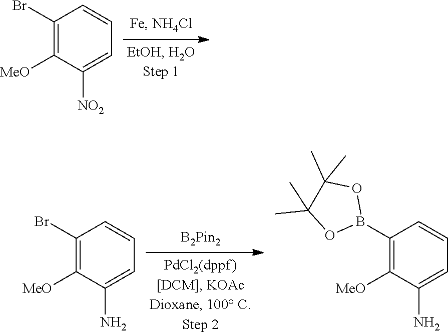 [SMILES: COc1c(Br)cccc1[N+](=O)[O-] ; COc1c(N)cccc1Br ; COc1c(N)cccc1B1OC(C)(C)C(C)(C)O1]

### Step 1

To a solution of 1-bromo-2-methoxy-3-nitrobenzene (2.0 g, 8.62 mmol) in ethanol (20 mL) and water (5 mL) was added iron (3.37 g, 60.3 mmol) and ammonium chloride (2.3 g, 43.3 mmol). The reaction was stirred at 60° C. for 3 h, diluted with ethanol (50 mL) and filtered through a celite pad. The filtrate was concentrated under reduced pressure to afford the crude product (2.5 g). The crude residue was diluted with EtOAc (100 mL), washed with water (2×20 mL) and brine (2×20 mL). The organic layer was dried over anhydrous sodium sulfate and concentrated under reduced pressure to afford 3-bromo-2-methoxyaniline (1.8 g, 8.55 mmol, 99% yield) as a brown liquid. MS (M+1) m/z: 202.0 (M+H)⁺. LC retention time 1.84 [A].

### Step 2

To a stirred solution of 3-bromo-2-methoxyaniline (1.80 g, 8.91 mmol) in 1,4-dioxane (15 mL) in a sealed tube was added bis(pinacolato)diborone (3.39 g, 13.36 mmol) and potassium acetate (2.62 g, 26.7 mmol). The reaction was purged with nitrogen gas for 5 min and then PdCl₂(dppf)·[DCM] (0.73 g, 0.89 mmol) was added. The reaction mixture was stirred at 90° C. for 5 h, then cooled to rt and diluted with EtOAc (100 mL). The reaction mixture was filtered through a celite pad and the filtrate was washed with water (2×50 mL) and brine (2×50 mL). Collected organic extract was dried over Na₂SO₄ and concentrated under reduced pressure to afford crude product, which was purified by silica gel column chromatography (25% ethyl acetate in pet ether) to afford 2-methoxy-3-(4,4,5,5-tetramethyl-1,3,2-dioxaborolan-2-yl)aniline (1.8 g, 6.88 mmol, 77% yield) as a pale brown solid. MS (M+1) m/z: 250.4 (M+H)⁺. LC retention time 2.11 [A].

### Intermediate 3

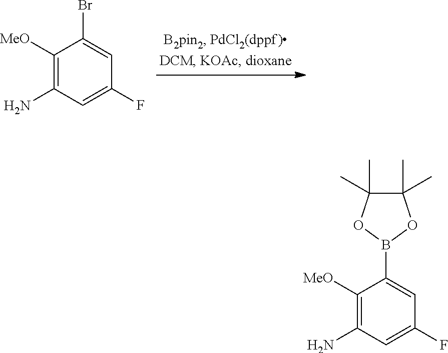 [SMILES: COc1c(N)cc(F)cc1Br ; COc1c(N)cc(F)cc1B1OC(C)(C)C(C)(C)O1]

A sealed vessel containing 3-bromo-5-fluoro-2-methoxyaniline (hydrochloride salt) (4.5 g, 17.5 mmol), 4,4,4′,4′,5,5,5′,5′-octamethyl-2,2′-bi(1,3,2,-dioxaborolane) (5.79 g, 22.8 mmol), PdCl₂(dppf)·DCM (0.716 g, 0.877 mmol) and potassium acetate (6.03 g, 61.4 mmol) in dioxane (100 mL) was heated to 105° C. Heating was continued overnight at which point the reaction was cooled to room temperature, absorbed onto Celite®, and dried under reduced pressure. The crude material was then purified using automated chromatography (solid loading) eluting with 0-50% EtOAc/hexanes. Product containing fractions were combined and concentrated under reduced pressure yielding 5-fluoro-2-methoxy-3-(4,4,5,5-tetramethyl-1,3,2-dioxaborolan-2-yl)aniline as a pale-yellow solid (2.55 g, 54% yield). MS (M+1) m/z: 268.3 (MH⁺). LC retention time 1.50 min [C].

### Intermediate 4

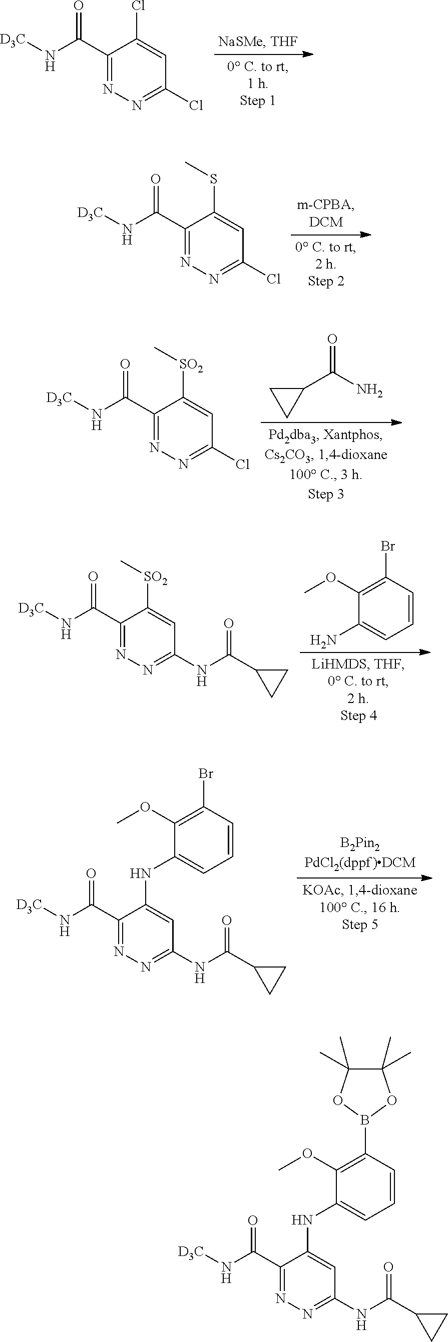

### Step 1

To a solution of 4,6-dichloro-N-(methyl-d₃)pyridazine-3-carboxamide (2.0 g, 9.57 mmol) in THF (80 mL) was added sodium thiomethoxide (1.0 g, 14.27 mmol) in water (25 mL) at 0° C. in 3 portions over a 5 minute interval, after the addition was complete the reaction was stirred at rt for 1 h. The reaction mixture was diluted with water (80 mL) and extracted with DCM (3×50 mL). Combined organic extracts were dried over Na₂SO₄ concentrated under vacuo and purified by silica gel column chromatography using 20% EtOAc in hexane to obtain 6-chloro-N-(methyl-d₃)-4-(methylthio)pyridazine-3-carboxamide (2.1 g, 8.74 mmol, 91% yield) as pale yellow solid. MS (M+1) m/z: 222.3 (M+H)⁺. LC retention time 0.71 min [F].

### Step 2

To a solution of 6-chloro-N-(methyl-d₃)-4-(methylthio)pyridazine-3-carboxamide (2.2 g, 9.97 mmol) in DCM (80 mL) was added m-CPBA (8.60 g, 49.8 mmol) at 0° C. and the reaction mixture was stirred at rt for 2 h under N₂ atmosphere. After completion of the reaction, it was quenched with saturated aqueous sodium bicarbonate solution (50 mL) and extracted with DCM (4×50 mL). The combined organic layers were dried over anhydrous Na₂SO₄, filtered and concentrated under reduced pressure. The crude residue was purified by silica gel column chromatography to obtain 6-chloro-N-(methyl-d₃)-4-(methylsulfonyl)pyridazine-3-carboxamide (1.5 g, 5.94 mmol, 59.5% yield) as white solid. ¹H NMR (DMSO d₆): 9.08 (s, 1H), 8.32 (s, 1H), 3.58 (s, 3H).

### Step 3

To a stirred solution of 6-chloro-N-(methyl-d₃)-4-(methylsulfonyl)pyridazine-3-carboxamide (1.1 g, 4.35 mmol) and cyclopropanecarboxamide (1.11 g, 13.06 mmol) in 1,4-dioxane (15 mL) was added Cs₂CO₃ (3.55 g, 10.88 mmol). The reaction mixture was degassed under N₂ for 10 min. Pd₂(dba)₃ (0.8 g, 0.871 mmol) and xantphos (0.50 g, 0.87 mmol) were added to the reaction mixture, which was then stirred at 100° C. for 3 h in a sealed tube. After completion, the reaction mixture was filtered through a celite pad and the filtrate was concentrated under reduced pressure. The crude product was purified by silica gel column chromatography (50% EtOAc in hexane) to obtain 6-(cyclopropanecarboxamido)-N-(methyl-d₃)-4-(methylsulfonyl)pyridazine-3-carboxamide (0.38 g, 0.95 mmol, 21.84% yield) as a yellow solid. MS (M+1) m/z: 302.1 (M+H)⁺. LC retention time 0.72 min [F].

### Step 4

To a solution of 6-(cyclopropanecarboxamido)-N-(methyl-d₃)-4-(methylsulfonyl)pyridazine-3-carboxamide (0.19 g, 0.627 mmol) and 3-bromo-2-methoxyaniline (0.13 g, 0.63 mmol) in THE (15 mL) was added LiHMDS (1.65 mL, 2.51 mmol, 1.5 M solution in THF) at 0° C. The reaction was warmed to rt and stirred for 2 h under nitrogen atmosphere. After completion of the reaction, the reaction mixture was quenched with saturated aqueous ammonium chloride solution (20 mL) and extracted with ethyl acetate (2×30 mL). The crude product was purified using silica gel column chromatography (60% EtOAc in hexane) to obtain 4-((3-bromo-2-methoxyphenyl)amino)-6-(cyclopropanecarboxamido)-N-(methyl-d₃)pyridazine-3-carboxamide (100 mg, 0.198 mmol, 31.6% yield) as a semi solid. MS (M+1) m/z: 424.8 (M+H)⁺. LC retention time 2.66 min [F].

### Step 5

To a stirred solution of 4-((3-bromo-2-methoxyphenyl)amino)-6-(cyclopropanecarboxamido)-N-(methyl-d₃)pyridazine-3-carboxamide (0.2 g, 0.47 mmol) and bis(pinacolato)diboron (0.24 g, 0.95 mmol) in 1,4-dioxane (15 mL) was added potassium acetate (0.12 mg, 1.18 mmol). The reaction mixture was degassed using N₂ gas for 10 minutes. PdCl₂(dppf)-DCM adduct (77 mg, 0.094 mmol) was added to the reaction mixture and the reaction warmed to 100° C. and stirred for 16 h. The reaction mixture was filtered through a celite pad and the filtrate was concentrated under reduced pressure. The crude residue was purified by silica gel column chromatography (60% ethyl acetate in hexane) to obtain 6-(cyclopropanecarboxamido)-4-((2-methoxy-3-(4,4,5,5-tetramethyl-1,3,2-dioxaborolan-2-yl)phenyl)amino)-N-(methyl-d₃)pyridazine-3-carboxamide (0.5 g, 0.276 mmol, 58.5% yield) as a brown syrup. MS (M+1) m/z: 471.1 (M+H)⁺. LC retention time 2.71 min [F].

### Intermediate 5

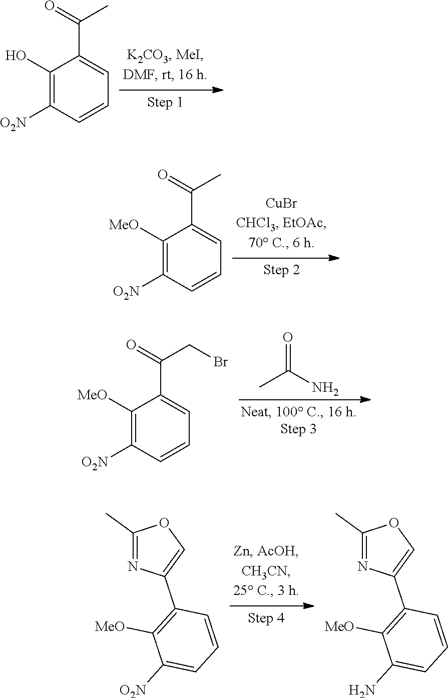

### Step 1

To a stirred solution of 1-(2-hydroxy-3-nitrophenyl)ethan-1-one (2.0 g, 11.04 mmol) in N,N-dimethylformamide (20 mL) was added K₂CO₃ (6.10 g, 44.2 mmol) stirring was continued for 30 min, at which point iodomethane (2.071 mL, 33.1 mmol) was added. The reaction mixture was stirred for at rt for an additional 16 h. After the completion of the reaction, cold water (100 mL) was added and the product extracted with EtOAc (2×100 mL). The combined organic layers were washed with brine (50 mL), dried over anhydrous Na₂SO₄, and concentrated under reduced pressure. The crude product was purified by column chromatography using silica gel 230-400 mesh (3% EtOAc in petroleum ether) to afford 1-(2-methoxy-3-nitrophenyl)ethan-1-one (2.0 g, 10.14 mmol, 92% yield) as a yellow solid. ¹H-NMR (400 MHz, DMSO-d₆): δ 7.95 (dd, J=1.60, 8.00 Hz, 1H), 7.84 (dd, J=1.60, 7.80 Hz, 1H), 7.31 (dd, J=3.60, 13.60 Hz, 1H), 3.97 (s, 3H), 2.69 (s, 3H).

### Step 2

A solution of 1-(2-methoxy-3-nitrophenyl)ethan-1-one (1.5 g, 7.69 mmol) in chloroform (10 mL) and ethyl acetate (10 mL) was cooled to 0° C. and then copper(II) bromide (2.06 g, 9.22 mmol) was added. The reaction was warmed to 70° C. and stirred for 6 h. After completion, the reaction mixture was filtered through a celite pad and washed with water (50 mL) and ethyl acetate (50 mL). The organic layer was separated, dried over anhydrous Na₂SO₄ and concentrated under reduced pressure to afford 2-bromo-1-(2-methoxy-3-nitrophenyl)ethan-1-one (1.1 g, 2.81 mmol, 36.6% yield) as a yellow solid. GCMS m/z: 272.9 (M)⁺. GC retention time 4.5 min [H].

### Step 3

2-Bromo-1-(2-methoxy-3-nitrophenyl)ethan-1-one (1.0 g, 3.65 mmol) in acetamide (4.31 g, 73.0 mmol) was stirred at 100° C. for 16 h. After completion, reaction mixture was diluted with water (50 mL) and EtOAc (50 mL). The organic layer was separated, dried over anhydrous Na₂SO₄, filtered and concentrated under reduced pressure to get crude residue. The crude product was purified by silica gel (100-200 mesh) column chromatography using 30% EtOAc in petroleum ether to afford 4-(2-methoxy-3-nitrophenyl)-2-methyloxazole (0.78 g, 2.60 mmol, 71.2% yield) as a brown liquid. MS (M+1) m/z: 235.0 (M+H)⁺. LC retention time 2.06 min [F].

### Step 4

To a stirred solution of 4-(2-methoxy-3-nitrophenyl)-2-methyloxazole (0.65 g, 2.78 mmol) in acetonitrile (1.5 mL) was added zinc (1.27 g, 19.43 mmol) and acetic acid (1.5 mL). The reaction mixture was stirred at 25° C. for 3 h. After completion the reaction mixture was filtered through a celite pad and washed with EtOAc (20 mL), dried over anhydrous Na₂SO₄ and concentrated under reduced pressure to afford 2-methoxy-3-(2-methyloxazol-4-yl)aniline (0.4 g, 1.665 mmol, 60.0% yield) as a brown liquid. MS (M+1) m/z: 205.2 (M+H)⁺. LC retention time 1.27 min [F].

### Intermediate 6

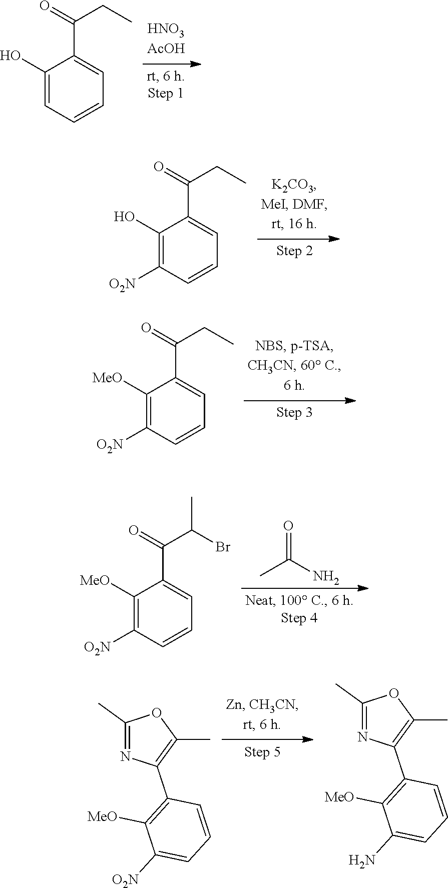

### Step 1

To a stirred solution of 1-(2-hydroxyphenyl)propan-1-one (1.0 g, 6.66 mmol) in acetic acid (1.91 ml, 33.3 mmol) was added nitric acid (0.387 ml, 8.66 mmol) in acetic acid (5 mL) dropwise. The reaction mixture was stirred at 25° C. for 6 h. After completion, the reaction mixture was quenched with cold water (200 mL) and the product extracted with ethyl acetate (2×100 mL). The combined organic extracts were washed with brine (100 mL) dried over anhydrous Na₂SO₄ and concentrated under reduce pressure. The crude product was purified by silica gel column chromatography to afford 1-(2-hydroxy-3-nitrophenyl)propan-1-one (0.7 g, 3.37 mmol, 50.6% yield) as a pale yellow solid. ¹H-NMR (400 MHz, CDCl₃) 13.25 (s, 1H), 8.14 (dd, J=1.60, 8.00 Hz, 2H), 7.05 (dd, J=8.00, Hz, 2H), 3.13 (q, J=7.20 Hz, 2H), 1.28 (t, J=7.20 Hz, 3H).

### Step 2

To a stirred solution of 1-(2-hydroxy-3-nitrophenyl)propan-1-one (2.0 g, 10.25 mmol) in N,N-dimethylformamide (20 mL) was added K₂CO₃ (5.66 g, 41.0 mmol) and iodomethane (1.922 mL, 30.7 mmol) at 25° C. The reaction mixture was stirred at 25° C. for 16 h. After completion the reaction mixture was diluted with cold water (100 mL) and extracted with EtOAc (2×100 mL). The combined organic layers were washed with brine (50 mL), dried over anhydrous Na₂SO₄ and concentrated under reduced pressure to get residue that was purified by column chromatography using silica gel 230-400 mesh (3% EtOAc in petroleum ether) to afford 1-(2-methoxy-3-nitrophenyl)propan-1-one (2.0 g, 9.18 mmol, 90% yield) as a yellow liquid. MS (M+1) m/z: 210.1 (M+H)⁺. LC retention time 1.86 min [G].

### Step 3

To a stirred solution of 1-(2-methoxy-3-nitrophenyl)propan-1-one (1.8 g, 8.60 mmol) in acetonitrile (3 mL) was added NBS (1.991 g, 11.19 mmol) and p-TsOH (0.164 g, 0.860 mmol). The reaction mixture was stirred for at 60° C. for 6 h. After completion the reaction mixture was diluted with water (100 mL) and extracted with EtOAc (2×50 mL). The combined organic layers were dried over anhydrous Na₂SO₄ and concentrated under reduced pressure to afford 2-bromo-1-(2-methoxy-3-nitrophenyl)propan-1-one (1.4 g, 4.13 mmol, 48.0% yield) as yellow solid. MS (M−1) m/z: 285.9 (M−H)⁺. LC retention time 2.74 min [F].

### Step 4

2-Bromo-1-(2-methoxy-3-nitrophenyl)propan-1-one (1.35 g, 4.69 mmol) in acetamide (5.54 g, 94 mmol) was stirred at 100° C. for 16 h. After completion, reaction mixture was diluted with water (50 mL) and extracted into ethyl acetate (2×50 mL). Collected organic layers were dried over anhydrous Na₂SO₄, filtered and concentrated under reduced pressure to obtain crude residue which was purified by column chromatography using silica gel 100-200 mesh (25% EtOAc in petroleum ether) to afford 4-(2-methoxy-3-nitrophenyl)-2,5-dimethyloxazole (1.1 g, 4.21 mmol, 90% yield) as a brown liquid. MS (M+1) m/z: 249.1 (M+H)⁺. LC retention time 2.17 min [G].

### Step 5

To a stirred solution of 4-(2-methoxy-3-nitrophenyl)-2,5-dimethyloxazole (1.0 g, 4.03 mmol) in acetonitrile (5 mL) was added zinc (1.844 g, 28.2 mmol) at 0° C. The reaction mixture was warmed to rt and stirred for 3 h. After completion, the reaction mixture was diluted with cold water (50 mL) and extracted into EtOAc (2×100 mL). The combined organic layers were washed with brine (100 mL), dried over anhydrous Na₂SO₄, filtered and concentrated under reduced pressure to afford 3-(2,5-dimethyloxazol-4-yl)-2-methoxyaniline (0.65 g, 2.73 mmol, 67.6% yield) as a brown liquid. MS (M+1) m/z: 219.0 (M+H)⁺. LC retention time 1.20 min [F].

### Example 1

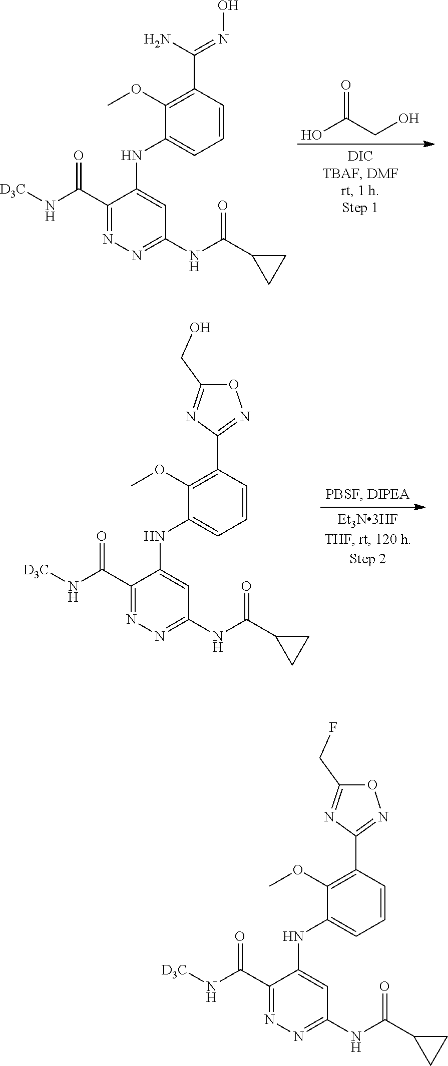 [SMILES: [2H]C([2H])([2H])NC(=O)c1nnc(NC(=O)C2CC2)cc1Nc1cccc(/C(N)=N/O)c1OC ; O=C(O)CO ; [2H]C([2H])([2H])NC(=O)c1nnc(NC(=O)C2CC2)cc1Nc1cccc(-c2noc(CO)n2)c1OC ; [2H]C([2H])([2H])NC(=O)c1nnc(NC(=O)C2CC2)cc1Nc1cccc(-c2noc(CF)n2)c1OC]

### Step 1

To a solution of (Z)-6-(cyclopropanecarboxamido)-4-((3-(N′-hydroxycarbamimidoyl)-2-methoxyphenyl)amino)-N-(methyl-d₃)pyridazine-3-carboxamide (0.18 g, 0.44 mmol) in DMF (1 mL) was added 2-hydroxyacetic acid (49.6 mg, 0.65 mmol) and DIC (0.14 mL, 0.87 mmol). The reaction mixture was stirred at rt for 1 h. To this reaction mixture was added TBAF (1.74 mL, 1.74 mmol, 1 M solution in THF) and the reaction was stirred at rt for 1 h. The reaction mixture was quenched with saturated aqueous NH₄Cl solution (10 mL) extracted with ethyl acetate (50 mL). The organic layer was dried over Na₂SO₄, filtered and concentrated under reduced pressure. The crude product was purified by silica gel column chromatography (10% MeOH in DCM) to afford 6-(cyclopropanecarboxamido)-4-((3-(5-(hydroxymethyl)-1,2,4-oxadiazol-3-yl)-2-methoxyphenyl)amino)-N-(methyl-d₃)pyridazine-3-carboxamide (105 mg, 54.6%) as an off-white solid. MS (M+1) m/z: 443.2 (M+H)⁺. LC retention time 1.67 min [F].

### Step 2

To a solution of 6-(cyclopropanecarboxamido)-4-((3-(5-(hydroxymethyl)-1,2,4-oxadiazol-3-yl)-2-methoxyphenyl)amino)-N-(methyl-d₃)pyridazine-3-carboxamide (0.1 g, 0.23 mmol) in THF (2.5 mL) was added perfluorobutanesulfonyl fluoride (0.081 mL, 0.452 mmol), DIPEA (0.237 mL, 1.356 mmol) and triethylamine trihydrofluoride (0.184 mL, 0.339 mmol, 30% solution) at rt. The reaction mixture was stirred at rt for 24 h. Another aliquot of perfluorobutanesulfonyl fluoride (0.081 mL, 0.452 mmol), DIPEA (0.237 mL, 1.356 mmol) and triethylamine trihydrofluoride (0.184 mL, 0.339 mmol, 30% solution) were added and stirred the reaction at rt for an additional 24 h. Added one more aliquot of reagents and stirring was continued at rt for 72 h. The reaction mixture was quenched with saturated aqueous NH₄Cl solution (10 mL) and extracted with ethyl acetate (2×25 mL). Combined organic extracts were collected, dried over anhydrous Na₂SO₄, filtered and concentrated under reduced pressure. The crude product was purified by prep HPLC to obtain desired product 6-(cyclopropanecarboxamido)-4-((3-(5-(fluoromethyl)-1,2,4-oxadiazol-3-yl)-2-methoxyphenyl)amino)-N-(methyl-d₃)pyridazine-3-carboxamide, formic acid salt (20 mg, 18% yield) as an off-white solid. MS (M+1) m/z: 445.2 (M+H)⁺. LC retention time 1.76 min [F].

¹H-NMR (400 MHz, DMSO-d₆): 11.37 (s, 1H), 11.05 (s, 1H), 9.18 (s, 1H), 8.16 (s, 1H), 7.75-7.70 (m, 2H), 7.40 (dd, J=5.20, 13.20 Hz, 1H), 5.98 (s, 1H), 5.83 (s, 1H), 3.75 (s, 3H), 2.09-2.08 (m, 1H), 0.84-0.81 (m, 4H).

### Example 2

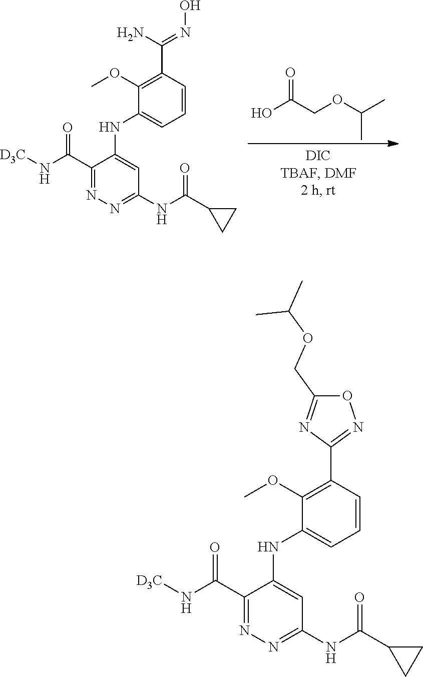

To a solution of (Z)-6-(cyclopropanecarboxamido)-4-((3-(N′-hydroxycarbamimidoyl)-2-methoxyphenyl)amino)-N-(methyl-d₃)pyridazine-3-carboxamide (0.2 g, 0.224 mmol) and 2-isopropoxyacetic acid (26.4 mg, 0.224 mmol) in DMF (0.3 mL) was added DIC (0.070 mL, 0.447 mmol). The reaction mixture was stirred at rt for 1 h. TBAF (0.67 mL, 0.67 mmol, 1M solution in THF) was added and stirring was continued at rt for another 1 h. The reaction mixture was quenched with saturated ammonium chloride solution (10 mL) and extracted with ethyl acetate (2×10 mL). The combined organic layers were dried over Na₂SO₄, filtered, and concentrated under reduced pressure. The crude compound was purified by prep HPLC to obtain desired 6-(cyclopropanecarboxamido)-4-((3-(5-(isopropoxymethyl)-1,2,4-oxadiazol-3-yl)-2-methoxyphenyl)amino)-N-(methyl-d₃)pyridazine-3-carboxamide, TFA (37 mg, 0.061 mmol, 27.4% yield) as an off-white solid. MS (M+1) m/z: 485.2 (M+H)⁺. LC retention time 2.41 min [F].

¹H-NMR (400 MHz, DMSO-d₆): δ 11.37 (s, 1H), 11.05 (s, 1H), 9.18 (s, 1H), 8.15 (s, 1H), 7.73-7.68 (m, 2H), 7.42-7.38 (m, 1H), 4.88 (s, 2H), 3.85-3.79 (m, 1H), 3.75 (s, 3H), 2.10-2.07 (m, 1H), 1.19-1.18 (m, 6H), 0.84-0.82 (m, 4H).

The following examples (3-4) were prepared in a similar manner to the preparation of example 2.

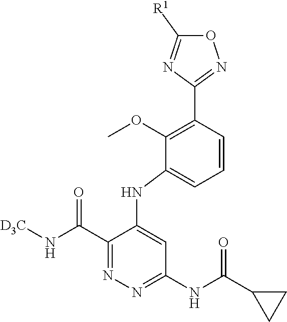

| Example | | | m/z | Rt (min) |
|---|---|---|---|---|
| No. | R¹ | MW | [M + H]⁺ | [Method] |
| 3 | 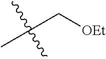 [SMILES: *COCC] | 470.5 | 471.2 | 2.19 [F] |
| 4 | 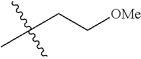 [SMILES: *CCOC] | 470.5 | 471.2 | 1.97 [F] |

### Example 5

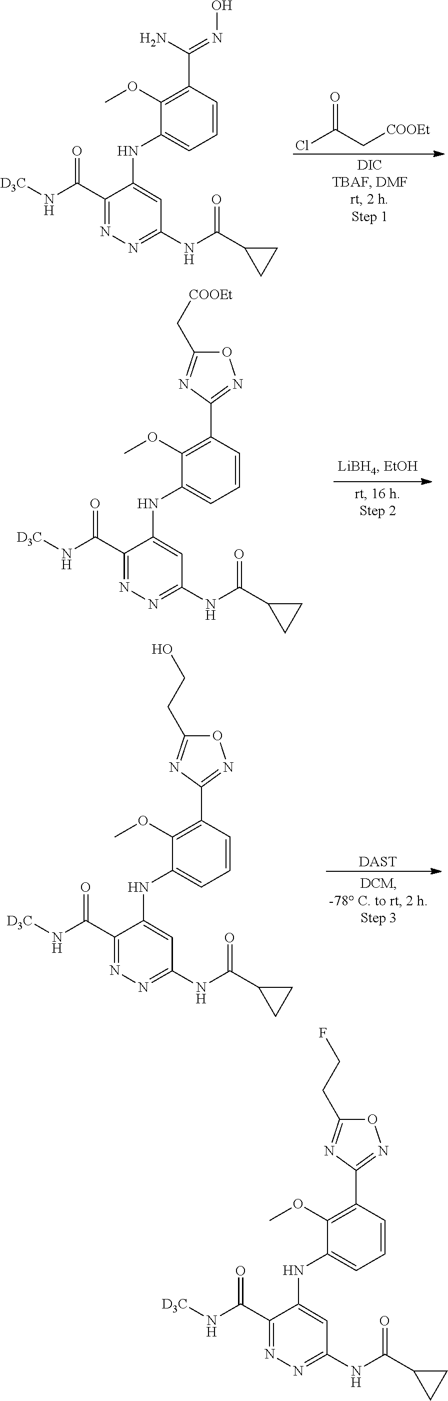 [SMILES: [2H]C([2H])([2H])NC(=O)c1nnc(NC(=O)C2CC2)cc1Nc1cccc(/C(N)=N/O)c1OC ; CCOC(=O)CC(=O)Cl ; [2H]C([2H])([2H])NC(=O)c1nnc(NC(=O)C2CC2)cc1Nc1cccc(-c2noc(CC(=O)OCC)n2)c1OC ; [2H]C([2H])([2H])NC(=O)c1nnc(NC(=O)C2CC2)cc1Nc1cccc(-c2noc(CCO)n2)c1OC ; "DAST" ; [2H]C([2H])([2H])NC(=O)c1nnc(NC(=O)C2CC2)cc1Nc1cccc(-c2noc(CCF)n2)c1OC]

### Step 1

To a solution of (Z)-6-(cyclopropanecarboxamido)-4-((3-(N′-hydroxycarbamimidoyl)-2-methoxyphenyl)amino)-N-(methyl-d₃)pyridazine-3-carboxamide (950 mg, 1.369 mmol) and 3-ethoxy-3-oxopropanoic acid (0.178 mL, 1.506 mmol) in DMF (5 mL) was added DIC (0.213 mL, 1.369 mmol). The reaction mixture was stirred at rt for 1 h. TBAF (1.369 mL, 1.369 mmol) was added and stirring was continued at rt for another 1 h. The reaction mixture was quenched with saturated ammonium chloride solution (20 mL) and extracted with ethyl acetate (2×50 mL). The combined organic layers were dried over Na₂SO₄, filtered, and concentrated under reduced pressure. The crude product was purified by silica gel column chromatography using ethyl acetate:hexane (4:1). column fractions were collected and solvent removed under reduced pressure. The pure product was isolated as an off-white solid. MS (M+1) m/z: 499.2 (M+H)⁺. LC retention time 2.1 min [F].

### Step 2

To a solution of ethyl 2-(3-(3-((6-(cyclopropanecarboxamido)-3-((methyl-d₃)carbamoyl)pyridazin-4-yl)amino)-2-methoxyphenyl)-1,2,4-oxadiazol-5-yl)acetate (280 mg, 0.562 mmol) in ethanol (5 mL) was added LiBH₄ (0.562 mL, 1.123 mmol) as a solution in THF at 0° C. The reaction mixture was warmed to rt and stirred for 16 h. The reaction was quenched via the addition of saturated aqueous NH₄Cl (10 mL). The ethanol was removed under reduced pressure. The crude product was partitioned between ethyl acetate (50 mL) and water (50 mL). The organic layer was collected, dried over anhydrous Na₂SO₄, filtered and concentrated. The crude product was purified by silica gel column chromatography using 10% MeOH:DCM to obtain the desired product as an off-white solid. MS (M+1) m/z: 457.2 (M+H)⁺. LC retention time 1.43 min [F].

### Step 3

To a stirred solution of 6-(cyclopropanecarboxamido)-4-((3-(5-(2-hydroxyethyl)-1,2,4-oxadiazol-3-yl)-2-methoxyphenyl)amino)-N-(methyl-d₃)pyridazine-3-carboxamide (70 mg, 0.153 mmol) in DCM (1 mL) was added DAST (0.041 mL, 0.307 mmol) in DCM (4 mL) at −78° C. under N₂. The reaction was stirred at this temperature for 1 h and then allowed to reach to rt, then stirred at rt for 1 h. The reaction mixture was quenched with saturated aqueous NH₄Cl solution (10 mL) and extracted with DCM (2×20 mL). Combined organic layer were dried, concentrated under reduced pressure and purified by prep HPLC to provide 6-(cyclopropanecarboxamido)-4-((3-(5-(2-fluoroethyl)-1,2,4-oxadiazol-3-yl)-2-methoxyphenyl)amino)-N-(methyl-d₃)pyridazine-3-carboxamide, formic acid salt (3 mg, 5.35 μmol, 3.49% yield) as an off white solid.

MS (M+1) m/z: 459.2 (M+H)⁺. LC retention time 2.30 min [F]. ¹H-NMR (400 MHz, MeOH-d₄): δ 8.25 (s, 1H), 7.83 (dd, J=1.60, 8.00 Hz, 1H), 7.73 (dd, J=1.60, 8.00 Hz, 1H), 7.39 (t, J=8.00 Hz, 1H), 4.99 (t, J=6.40 Hz, 1H), 4.91 (t, J=17.60 Hz, 1H), 3.85 (s, 3H), 3.51 (t, J=1.60 Hz, 1H), 3.48 (t, J=13.20 Hz, 1H), 1.97-1.93 (m, 1H), 0.95-0.91 (m, 4H).

### Example 6

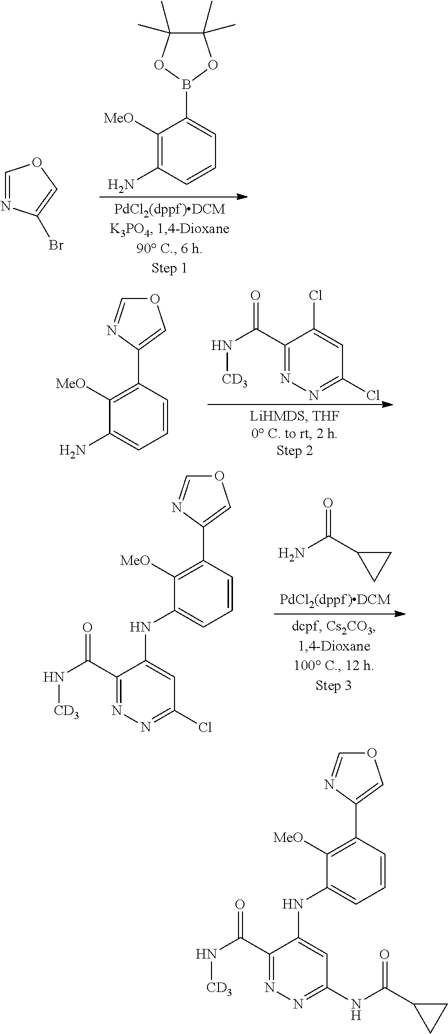

### Step 1

To the stirred solution of 4-bromooxazole·HCl (300 mg, 1.627 mmol) and 2-methoxy-3-(4,4,5,5-tetramethyl-1,3,2-dioxaborolan-2-yl)aniline (446 mg, 1.790 mmol) in 1,4-dioxane (10 mL) was added aqueous 2N K₃PO₄ (2.440 mL, 4.88 mmol) at ambient temperature and the reaction was purged under N₂ gas for 10 min. PdCl₂(dppf)·DCM (133 mg, 0.163 mmol) was subsequently added to the reaction mixture and the reaction was warmed to 90° C. and stirred for 6 h in a sealed tube. After completion, the reaction mixture was diluted with ethyl acetate (30 mL) and filtered through a celite pad, washing with ethyl acetate (50 mL). The filtrate was concentrated under reduced pressure to afford crude product. The crude product was purified by silica gel (100-200 mesh) column chromatography using 40-45% of EtOAc in petroleum ether as an eluent to afford desired product 2-methoxy-3-(oxazol-4-yl)aniline (220 mg, 1.073 mmol, 66.0% yield) as a brown solid. MS (M+1) m/z: 191.0 (M+H)⁺. LC retention time 1.05 min [F].

### Step 2

To the stirred solution of 4,6-dichloro-N-(methyl-d₃)pyridazine-3-carboxamide (130 mg, 0.622 mmol) and 2-methoxy-3-(oxazol-4-yl)aniline (130 mg, 0.684 mmol) in THE (10 mL) was added a 1 M solution of LiHMDS in THE (2.488 mL, 2.488 mmol) dropwise at 0° C. Reaction mixture was slowly warmed to ambient temperature and stirred for 2 h at which point it was quenched with saturated aqueous NH₄Cl solution and extracted with EtOAc (2×100 mL). Combined organic layers were washed with water (50 mL) and brine (50 mL), dried over anhydrous Na₂SO₄ and concentrated under reduced pressure. The crude product was purified by silica gel column chromatography using 30-35% EtOAc in petroleum ether as an eluent to afford 6-chloro-4-((2-methoxy-3-(oxazol-4-yl)phenyl)amino)-N-(methyl-d₃)pyridazine-3-carboxamide (140 mg, 0.367 mmol, 59.0% yield) as an off white solid. MS (M+1) m/z: 363.0 (M+H)⁺. LC retention time 2.33 min [F].

### Step 3

To the stirred solution of 6-chloro-4-((2-methoxy-3-(oxazol-4-yl)phenyl)amino)-N-(methyl-d₃)pyridazine-3-carboxamide (120 mg, 0.331 mmol) and cyclopropanecarboxamide (84 mg, 0.992 mmol) in 1,4-dioxane (3 mL) was added Cs₂CO₃ (269 mg, 0.827 mmol) and the vessel purged under N₂ for 10 min. 1,1′-bis(di-cyclohexylphosphino)ferrocene (38.3 mg, 0.066 mmol) and Pd₂dba₃ (30.3 mg, 0.033 mmol) were added to the reaction mixture and the vessel was sealed. The reaction was stirred at 100° C. for 12 h. Reaction mixture was diluted with ethyl acetate (20 mL), filtered through a celite pad and washed with ethyl acetate (50 mL). The filtrate was concentrated under reduced pressure and purified by silica gel column chromatography (100-200 mesh) using 85-90% EtOAc in petroleum ether as an eluent to afford product 6-(cyclopropanecarboxamido)-4-((2-methoxy-3-(oxazol-4-yl)phenyl)amino)-N-(methyl-d₃)pyridazine-3-carboxamide (43.74 mg, 0.106 mmol, 32.0% yield) as a white solid. MS (M+1) m/z: 412.0 (M+H)⁺. LC retention time 1.92 min [F]. ¹H-NMR (400 MHz, DMSO-d₆): 11.33 (s, 1H), 10.97 (s, 1H), 9.16 (s, 1H), 8.55-8.52 (m, 1H), 8.12 (s, 1H), 7.84-7.82 (m, 1H), 7.44-7.42 (m, 1H), 7.33-7.29 (m, 1H), 3.70 (s, 3H), 2.39-2.33 (m, 1H), 2.11-2.07 (m, 4H).

### Example 7

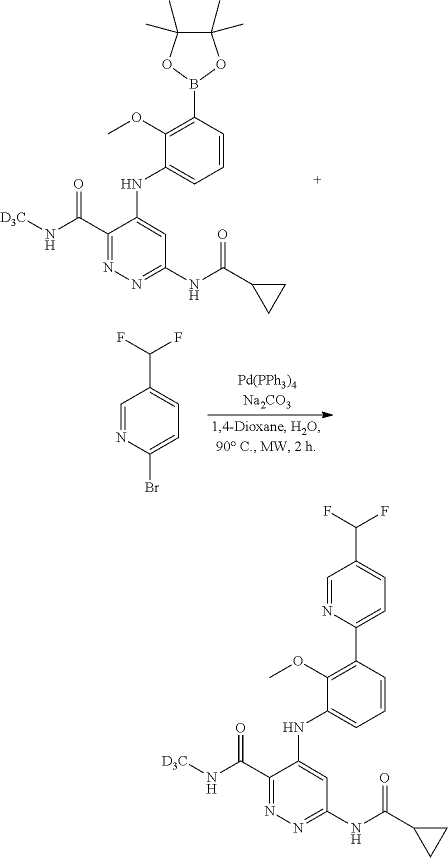 [SMILES: [2H]C([2H])([2H])NC(=O)c1nnc(NC(=O)C2CC2)cc1Nc1cccc(B2OC(C)(C)C(C)(C)O2)c1OC ; FC(F)c1ccc(Br)nc1 ; [2H]C([2H])([2H])NC(=O)c1nnc(NC(=O)C2CC2)cc1Nc1cccc(-c2ccc(C(F)F)cn2)c1OC]

To a stirred solution of 6-(cyclopropanecarboxamido)-4-((2-methoxy-3-(4,4,5,5-tetramethyl-1,3,2-dioxaborolan-2-yl)phenyl)amino)-N-(methyl-d₃)pyridazine-3-carboxamide (100 mg, 0.21 mmol) and 2-bromo-5-(difluoromethyl)pyridine (0.044 g, 0.213 mmol) in 1,4-dioxane (2 mL) and H₂O (0.5 mL) was added Na₂CO₃ (45.0 mg, 0.425 mmol). The reaction mixture was degassed under N₂ gas for 10 min. Tris(triphenylphosphine)palladium (25.0 mg, 0.021 mmol) was added to the reaction mixture which was then heated to 90° C. for 2 h under microwave irradiation. After completion, the reaction was filtered through a celite pad and the filtrate concentrated under reduced pressure. The crude product was purified by prep HPLC to obtain methyl 6-(cyclopropanecarboxamido)-4-((3-(5-(difluoromethyl)pyridin-2-yl)-2-methoxyphenyl)amino)-N-(methyl-d₃)pyridazine-3-carboxamide, formic acid salt (15 mg, yield 13.5%) as an off-white solid. MS (M+1) m/z: 472.2 (M+H)⁺. LC retention time 2.54 min [F]. ¹H NMR (400 MHz, DMSO-d6): δ 11.36 (s, 1H), 11.01 (s, 1H), 9.17 (s, 1H), 8.92 (s, 1H), 8.13 (s, 1H), 8.11-8.11 (m, 1H), 8.01-8.03 (m, 1H), 7.56-7.59 (m, 2H), 7.33-7.37 (m, 1H), 7.16 (s, 1H), 3.50 (s, 3H), 2.07-2.13 (m, 1H), 0.83-0.88 (m, 4H).

The following examples 8-13 were prepared in a similar manner to the preparation of example 7.

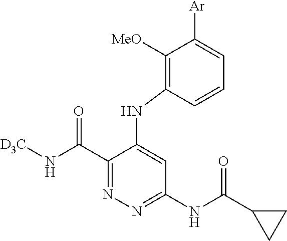

| Example | | | m/z | Rt (min) |
|---|---|---|---|---|
| No. | Ar | MW | [M + H]⁺ | [Method] |
| 8 | 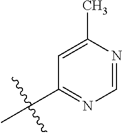 [SMILES: *c1cc(C)ncn1] | 436.49 | 437.2 | 1.70 [F] |
| 9 | 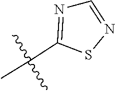 [SMILES: *c1ncns1] | 428.485 | 429.0 | 2.03 [F] |
| 10 | 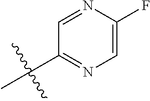 [SMILES: *c1cnc(F)cn1] | 440.18 | 441.2 | 2.49 [F] |
| 11 | 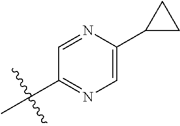 [SMILES: *c1cnc(C2CC2)cn1] | 462.53 | 463.2 | 2.74 [F] |
| 12 | 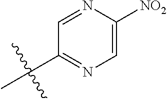 [SMILES: *c1cnc([N+](=O)[O-])cn1] | 467.46 | 468.0 | 2.23 [F] |
| 13 | 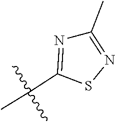 [SMILES: *c1nc(C)ns1] | 442.51 | 443.2 | 2.47 [F] |

### Example 14

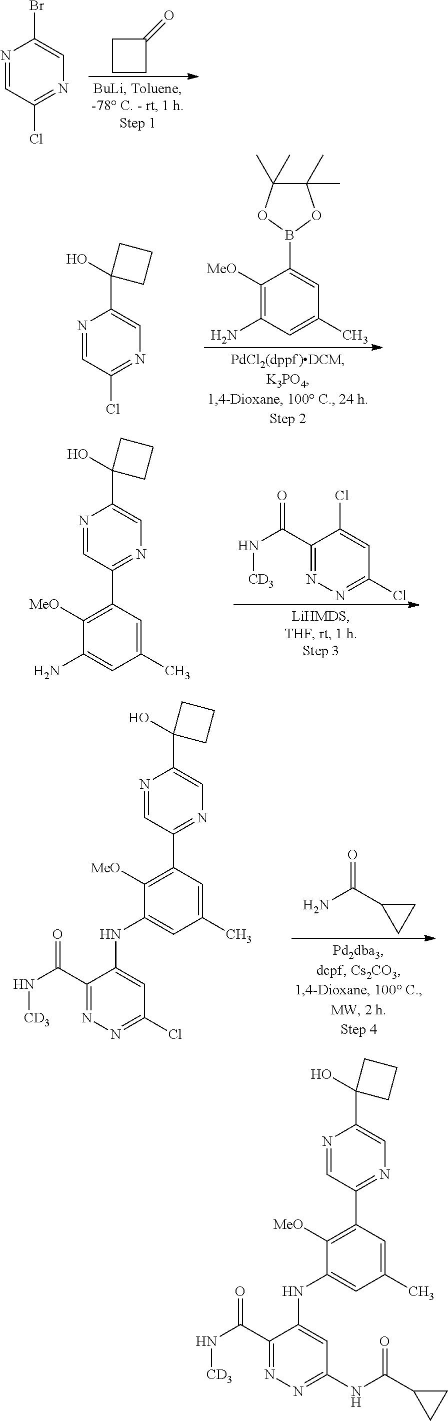

### Step 1

To a stirred solution of 2-bromo-5-chloropyrazine (1.0 g, 5.17 mmol) in toluene (15 mL) under N2 atmosphere was added n-butyllithium (2.58 mL, 5.17 mmol, 2M solution in hexane) at −78° C. The reaction solution became dark red and was allow to stir an additional 20 min at −78° C. To this solution cyclobutanone (0.4 g, 5.69 mmol) in toluene (2 mL) was added dropwise and the reaction stirred for 30 mins at −78° C. The reaction was allowed to reach rt and stirred for about 1 h. The reaction mixture was quenched with saturated aqueous NH₄Cl solution (20 mL) and extracted with ethyl acetate (2×30 mL). The combined organic layers were dried over anhydrous Na₂SO₄. The crude residue was purified by silica gel column chromatography (10% EtOAc in hexane) to obtain 1-(5-chloropyrazin-2-yl)cyclobutan-1-ol (800 mg, 4.33 mmol, 84% yield) as an orange oil. GCMS (M) m/z: 184.1 (M)⁺. GC retention time 7.43 min [H].

### Step 2

To a stirred solution of 1-(5-chloropyrazin-2-yl)cyclobutan-1-ol (500 mg, 2.71 mmol) in 1,4-dioxane (30 mL) was added 2-methoxy-5-methyl-3-(4,4,5,5-tetramethyl-1,3,2-dioxaborolan-2-yl)aniline (750 mg, 2.85 mmol) and 2M K₃PO₄ (5 mL, 10.00 mmol). The reaction mixture was purged under N₂ for 5 min and PdCl₂(dppf)·DCM (200 mg, 0.245 mmol) was added. The reaction mixture was sealed and stirred at 90° C. for 16 h, then cooled to rt and diluted with ethyl acetate (100 mL). The reaction mixture was filtered through a celite pad, and the filtrate was washed with water (2×50 mL) and brine (2×50 mL). Collected organic extract was dried over Na₂SO₄ and concentrated under reduced pressure to afford crude product which was purified by silica gel column chromatography (30% EtOAc in petroleum ether) to afford 1-(5-(3-amino-2-methoxy-5-methylphenyl)pyrazin-2-yl)cyclobutan-1-ol (440 mg, 1.542 mmol, 56.9% yield) as a pale brown semi solid. MS (M+1) m/z: 286.2 (M+H)⁺. LC retention time 1.42 min [F]

### Step 3

To a stirred solution of 4,6-dichloro-N-(methyl-d₃)pyridazine-3-carboxamide (250 mg, 1.196 mmol) and 1-(5-(3-amino-2-methoxy-5-methylphenyl)pyrazin-2-yl)cyclobutan-1-ol (300 mg, 1.051 mmol) in THE (10 mL) was added LiHMDS (3.6 mL, 3.60 mmol, 1M solution in THF) at 0° C. The reaction mixture was stirred at 0° C. for 4 h and then allowed to reach to rt. The reaction mixture was quenched with saturated NH₄Cl solution (20 mL) and the product extracted with ethyl acetate (2×50 mL). The combined organic layers were washed with brine (25 mL), dried over anhydrous Na₂SO₄, filtered and concentrated. The crude product was purified by silica gel column chromatography using 70% EtOAc in petroleum ether to obtain 6-chloro-4-((3-(5-(1-hydroxycyclobutyl)pyrazin-2-yl)-2-methoxy-5-methylphenyl)amino)-N-(methyl-d₃)pyridazine-3-carboxamide (200 mg, 0.336 mmol, 28.1% yield) as a pale-yellow solid. MS (M+1) m/z: 458.2 (M+H)⁺. LC retention time 2.26 min [F]

### Step 4

To a stirred solution of 6-chloro-4-((3-(5-(1-hydroxycyclobutyl)pyrazin-2-yl)-2-methoxy-5-methylphenyl)amino)-N-(methyl-d₃)pyridazine-3-carboxamide (200 mg, 0.437 mmol) in 1,4-dioxane (10 mL) in a microwave vial and was added cyclopropanecarboxamide (37.2 mg, 0.437 mmol) and Cs₂CO₃ (427 mg, 1.310 mmol). The reaction mixture was degassed under N₂ for 10 min, then 1,1′-bis(dicyclohexylphosphino)ferrocene (50.5 mg, 0.087 mmol) and Pd₂(dba)₃ (40.0 mg, 0.044 mmol) were added and the vessel degassed (via N₂) for another 10 min. The reaction vessel was sealed and stirred under microwave irradiation at 100° C. for 2 h. The reaction mixture was filtered through a celite pad and washed with ethyl acetate (20 mL). The filtrate was concentrated under reduced pressure and the crude compound was purified by silica gel (230-400 mesh) column chromatography using 50-60% EtOAc in hexane as an eluent to afford desired 6-(cyclopropanecarboxamido)-4-((3-(5-(1-hydroxycyclobutyl)pyrazin-2-yl)-2-methoxy-5-methylphenyl)amino)-N-(methyl-d₃)pyridazine-3-carboxamide (26 mg, 0.050 mmol, 11.52% yield) as a pale-yellow solid. MS (M+1) m/z: 507.2 (M+H)⁺. LC retention time 2.14 min [F]. ¹H-NMR (400 MHz, DMSO-d6): δ 11.33 (s, 1H), 10.91 (s, 1H), 9.16 (s, 1H), 9.02 (s, 1H), 8.91 (s, 1H), 8.14 (s, 1H), 7.41 (d, J=8.80 Hz, 1H), 6.06 (s, 2H), 3.50 (s, 3H), 2.68-2.61 (m, 3H), 2.53-2.50 (m, 2H), 2.35 (d, J=1.60 Hz, 2H), 2.34-2.33 (m, 1H), 2.08 (m, 1H), 1.86 (m, 1H), 0.84 (s, 4H).

The following example 15 was prepared in a similar manner to the preparation of example 14.

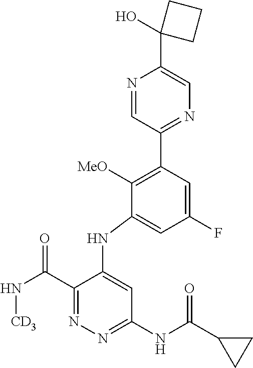 [SMILES: [2H]C([2H])([2H])NC(=O)c1nnc(NC(=O)C2CC2)cc1Nc1cc(F)cc(-c2cnc(C3(O)CCC3)cn2)c1OC]

| Example No. | MW | m/z [M + H]⁺ | Rt (min) [Method] |
|---|---|---|---|
| 15 | 510.54 | 511.2 | 2.49 [F] |

### Example 16

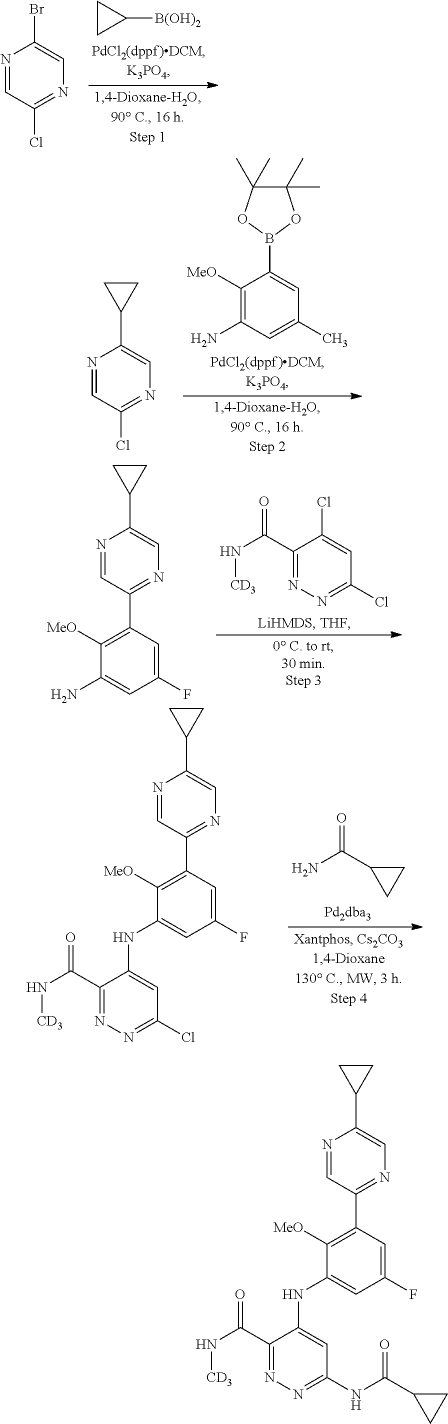

### Step 1

To a stirred solution of 2-bromo-5-chloropyrazine (2.5 g, 12.92 mmol) in 1,4-dioxane (40 mL) and water (10 mL) was added cyclopropylboronic acid (1.11 g, 12.92 mmol) and K₃PO₄ (8.23 g, 38.8 mmol) followed by PdCl₂(dppf)·DCM (0.528 g, 0.646 mmol). The reaction mixture was degassed under N₂ for 10 min. The resultant reaction mixture was heated to 90° C. for 16 h and, after completion of the reaction, the reaction mixture was diluted with ethyl acetate (100 mL), filtered through a celite pad and washed with ethyl acetate (100 mL). The filtrate was washed with water (100 mL) followed by brine (100 mL). The organic layer was dried over Na₂SO₄ and filtered. Solvent was evaporated under reduced pressure and the resultant crude residue was purified by column chromatography on silica gel (230-400 mesh) using 5-10% EtOAc in petroleum ether to afford 2-chloro-5-cyclopropylpyrazine (800 mg, 5.17 mmol, 40.0% yield) as a pale yellow solid.

¹H-NMR (400 MHz, DMSO-d₆): δ 8.28 (d, J=1.20 Hz, 1H), 8.41 (dd, J=1.60, 5.80 Hz, 1H), 2.10-2.04 (m, 1H), 1.13-1.07 (m, 4H).

### Step 2

To a stirred solution of 5-fluoro-2-methoxy-3-(4,4,5,5-tetramethyl-1,3,2-dioxaborolan-2-yl)aniline (0.5 g, 1.872 mmol) in 1,4-dioxane (20 mL) and water (5 mL) in a 100 mL sealed tube were added 2-chloro-5-cyclopropylpyrazine (0.405 g, 2.62 mmol) and K₃PO₄ (1.192 g, 5.62 mmol) followed by PdCl₂(dppf)·DCM (0.076 g, 0.094 mmol). The reaction mixture was degassed under N₂ for 10 min. The resultant reaction mixture was heated to 90° C. for 16 h. The reaction mixture was diluted with ethyl acetate (100 mL), filtered through a celite pad and washed with ethyl acetate (100 mL). The filtrate was washed with water (100 mL) followed by brine (100 mL), dried over Na₂SO₄, filtered and concentrated under reduced pressure. The resultant crude residue was purified by column chromatography on silica gel (230-400 mesh) using 5-10% EtOAc in petroleum ether to afford 3-(5-cyclopropylpyrazin-2-yl)-5-fluoro-2-methoxyaniline (0.12 g, 0.435 mmol, 23.24% yield) as a pale yellow liquid. MS (M+1) m/z: 260.2 (M+H)⁺. LC retention time 2.43 min [F].

### Step 3

To a solution of 3-(5-cyclopropylpyrazin-2-yl)-5-fluoro-2-methoxyaniline (0.12 g, 0.463 mmol) in THE (5 mL) was added 4,6-dichloro-N-(methyl-d₃)pyridazine-3-carboxamide (0.116 g, 0.555 mmol) followed by LiHMDS (1.157 mL, 1.157 mmol, 1M solution in THF) dropwise over 5 min at 0° C. The reaction was stirred at rt for 30 min and then quenched with saturated aqueous NH₄Cl solution (15 mL). The reaction mixture was partitioned between EtOAc (100 mL) and water (50 mL). The organic layer was washed with brine solution (50 mL), dried over anhydrous Na₂SO₄, filtered and concentrated to afford a brown solid. The resultant crude product was triturated with n-pentane:diethyl ether (1:1) to give 6-chloro-4-((3-(5-cyclopropylpyrazin-2-yl)-5-fluoro-2-methoxyphenyl)amino)-N-(methyl-d3)pyridazine-3-carboxamide (0.13 g, 0.217 mmol, 46.8% yield) as an off-white solid. MS (M+1) m/z: 432.2 (M+H)⁺. LC retention time 2.81 min [F].

### Step 4

To a stirred solution of 6-chloro-4-((3-(5-cyclopropylpyrazin-2-yl)-5-fluoro-2-methoxyphenyl)amino)-N-(methyl-d₃)pyridazine-3-carboxamide (0.15 g, 0.347 mmol) in 1,4-dioxane (10 mL) in a 20 mL microwave vial were added cyclopropanecarboxamide (0.03 g, 0.347 mmol), Pd₂(dba)₃ (0.016 g, 0.017 mmol), xantphos (10.05 mg, 0.017 mmol) and Cs₂CO₃ (0.113 g, 0.347 mmol). The reaction mixture was degassed with N₂ for 5 min. The resultant reaction mixture was heated to 130° C. for 3 h under microwave irradiation. The reaction mixture was diluted with ethyl acetate (100 mL), filtered through a celite pad and washed with ethyl acetate (100 mL). The filtrate was washed with water (100 mL) and brine (100 mL) and dried over Na₂SO₄. Solvent was evaporated under reduced pressure and the resultant crude residue was purified by reverse phase column chromatography (c18 column) using 70% acetonitrile in 0.1% aqueous ammonium acetate solution to afford 6-(cyclopropanecarboxamido)-4-((3-(5-cyclopropylpyrazin-2-yl)-5-fluoro-2-methoxyphenyl)amino)-N-(methyl-d₃)pyridazine-3-carboxamide (50 mg, 0.100 mmol, 28.8% yield) as an off-white solid. MS (M+1) m/z: 481.1 (M+H)⁺. LC retention time 2.14 min [G]. ¹H-NMR (400 MHz, DMSO-d6): δ 11.42 (s, 1H), 11.17 (s, 1H), 9.21 (s, 1H), 8.95 (d, J=0.80 Hz, 1H), 8.77 (d, J=0.80 Hz, 1H), 8.27 (s, 1H), 7.49-7.45 (m, 1H), 7.37-7.34 (m, 1H), 3.52 (s, 3H), 2.31-2.27 (m, 1H), 2.12-2.09 (m, 1H), 1.14-1.09 (m, 2H), 1.06-1.03 (m, 2H), 0.86 (d, J=6.00 Hz, 4H).

### Example 17

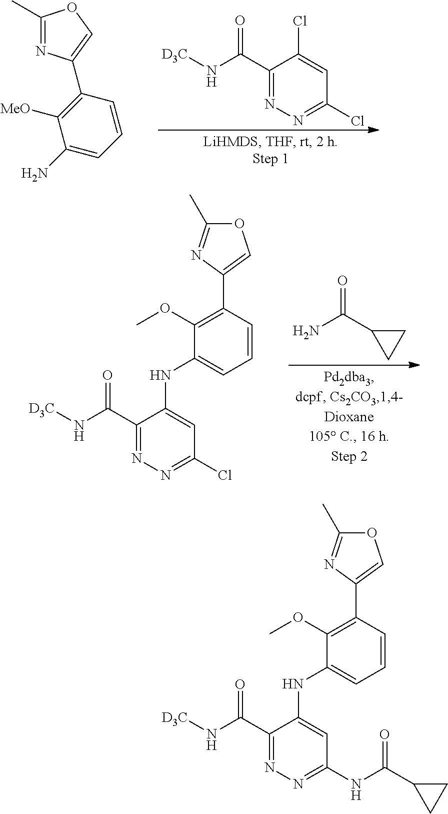 [SMILES: COc1c(N)cccc1-c1coc(C)n1 ; [2H]C([2H])([2H])NC(=O)c1nnc(Cl)cc1Cl ; [2H]C([2H])([2H])NC(=O)c1nnc(Cl)cc1Nc1cccc(-c2coc(C)n2)c1OC ; NC(=O)C1CC1 ; [2H]C([2H])([2H])NC(=O)c1nnc(NC(=O)C2CC2)cc1Nc1cccc(-c2coc(C)n2)c1OC]

### Step 1

To a stirred solution of 4,6-dichloro-N-(methyl-d₃)pyridazine-3-carboxamide (1.0 g, 4.78 mmol) and 2-methoxy-3-(2-methyloxazol-4-yl)aniline (0.586 g, 2.87 mmol) in THF (20 mL) was added LiHMDS (9.57 mL, 14.35 mmol, 1M Solution in THF) at rt. The reaction mixture was stirred at rt for 2 h. After the completion of the reaction the reaction mixture was diluted with 50 mL water and extracted with ethyl acetate (2×50 mL). Combined organic layers were dried over anhydrous Na₂SO₄ and concentrated under reduced pressure to afford crude product. The crude residue was purified by column chromatography using silica gel 100-200 mesh (30% EtOAc in petroleum ether) to afford 6-chloro-4-((2-methoxy-3-(2-methyloxazol-4-yl)phenyl)amino)-N-(methyl-d₃) pyridazine-3-carboxamide (0.66 g, 1.156 mmol, 24.17% yield) as a brown solid. MS (M+1) m/z: 377.0 (M+H)⁺. LC retention time 2.12 min [F].

### Step 2

To a solution of 6-chloro-4-((2-methoxy-3-(2-methyloxazol-4-yl)phenyl)amino)-N-(methyl-d₃) pyridazine-3-carboxamide (0.2 g, 0.531 mmol) in 1,4-dioxane (10 mL) was added cyclopropanecarboxamide (0.090 g, 1.062 mmol), Cs₂CO₃ (0.346 g, 1.062 mmol) and 1,1′-bis(dicyclohexylphosphino)ferrocene (0.061 g, 0.106 mmol). The vessel was purged under N₂ for 10 min and Pd₂(dba)₃ (0.097 g, 0.106 mmol) was added. The vessel was heated to 105° C. and stirred for 16 h. The reaction mixture was filtered through a celite pad and washed with ethyl acetate (50 mL). The collected filtrate was concentrated under reduced pressure and purified by reverse phase preparatory HPLC to afford 6-(cyclopropanecarboxamido)-4-((2-methoxy-3-(2-methyloxazol-4-yl)phenyl)amino)-N-(methyl-d₃)pyridazine-3-carboxamide (0.020 g, 0.043 mmol, 8.09% yield) as an off-white solid. MS (M+1) m/z: 426.0 (M+H)⁺. LC retention time 1.94 min [F]. ¹H-NMR (400 MHz, DMSO-d₆): δ 11.33 (s, 1H), 10.96 (s, 1H), 9.17 (s, 1H), 8.38 (s, 1H), 8.12 (s, 1H), 7.78 (d, J=1.60 Hz, 1H), 7.40 (d, J=1.20 Hz, 1H), 7.26-7.30 (m, 1H), 3.68 (s, 3H), 2.51 (s, 3H), 2.09-2.07 (m, 1H), 0.83-0.81 (m, 4H).

The following example 18 was prepared from intermediate 4 in a similar manner to the preparation of example 17.

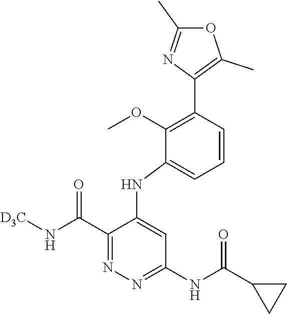 [SMILES: [2H]C([2H])([2H])NC(=O)c1nnc(NC(=O)C2CC2)cc1Nc1cccc(-c2nc(C)oc2C)c1OC]

| Example | | m/z | Rt (min) |
|---|---|---|---|
| No. | MW | [M + H]⁺ | [Method] |
| 18 | 439.49 | 440.0 | 1.97 [F] |

### Example 19

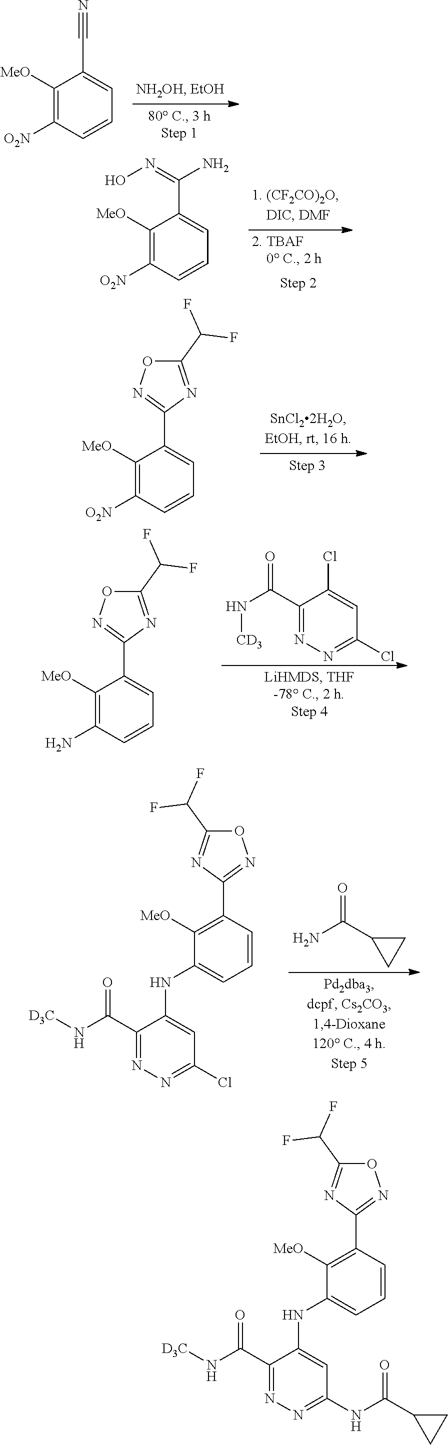

### Step 1

To a stirred solution of 2-methoxy-3-nitrobenzonitrile (3 g, 16.84 mmol) in ethanol (50 mL) was added hydroxyl amine (50% in water) (2.223 g, 33.7 mmol). The reaction mixture was allowed to stir at 80° C. for 3 h. After completion the reaction mixture was concentrated under reduced pressure and quenched with ice cold water (50 mL). The crude product was extracted with ethyl acetate (2×100 mL) and the combined organic layers were washed with water (100 mL) and brine (100 mL) and dried over Na₂SO₄. The organic solvent was evaporated under reduced pressure and the resultant crude product was triturated with diethyl ether to get desired (Z)—N′-hydroxy-2-methoxy-3-nitrobenzimidamide (3.00 g, 11.36 mmol, 67.5% yield) as a brown solid. MS (M+1) m/z: 212.1 (M+H)⁺. LC retention time 1.04 min [G].

### Step 2

To a stirred solution of (E)-N′-hydroxy-2-methoxy-3-nitrobenzimidamide (2.50 g, 11.8 mmol) in DMF (50 mL) was added N,N-diisopropylcarbodiimide (2.99 g, 23.68 mmol) and difluoro acetic anhydride (1.089 g, 11.84 mmol) at 0° C. The reaction mixture was warmed to rt and stirred for 1 h, then TBAF (11.84 mL, 11.84 mmol) was added and stirring continued for 1 h. The reaction mixture was concentrated and partitioned between water (50 mL) and ethyl acetate (100 mL). The organic layer was separated, washed with brine (100 mL) and then dried over Na₂SO₄. The organic solvent was evaporated under reduced pressure and the resultant crude product was purified by silica gel column chromatography by using 100-200 mesh to obtain desired 5-(difluoromethyl)-3-(2-methoxy-3-nitrophenyl)-1,2,4-oxadiazole (3.0 g, 10.51 mmol, 89% yield) as a brown solid.

MS (M+1) m/z: 273.0 (M+H)⁺. LC retention time 2.27 min [F].

### Step 3

To a stirred solution of 5-(difluoromethyl)-3-(2-methoxy-3-nitrophenyl)-1,2,4-oxadiazole (1.5 g, 5.53 mmol) in ethanol (25 mL) was added tin(II) chloride dihydrate (2.496 g, 11.06 mmol). The reaction mixture was stirred at rt for 16 h. After completion of the reaction, the reaction mixture was filtered through a celite pad and washed with methanol (100 mL). The filtrate was concentrated and partitioned between ethyl acetate (100 mL) and water (50 mL). The organic layer was washed with water (100 mL) and brine (100 mL), and dried over Na₂SO₄. The Na₂SO₄ was removed via filtration and the filtrate was concentrated under reduced pressure to afford the crude product 3-(5-(difluoromethyl)-1,2,4-oxadiazol-3-yl)-2-methoxyaniline (1.0 g, 3.48 mmol, 63.0% yield) which was used in subsequent steps without further purification. MS (M+1) m/z: 242.2 (M+H)⁺. LC retention time 2.01 min [F].

### Step 4

To a stirred solution of 4,6-dichloro-N-(methyl-d₃)pyridazine-3-carboxamide (500 mg, 2.392 mmol) in THE (15 mL) was added 3-(5-(difluoromethyl)-1,2,4-oxadiazol-3-yl)-2-methoxyaniline (692 mg, 2.87 mmol). The mixture was cooled to −78° C. and then LiHMDS (7.18 mL, 7.18 mmol, 1M solution in THF) was added. The reaction mixture was stirred for 2 h. After completion of the reaction, the reaction mixture was quenched with ice cold water (100 mL) and the product extracted with ethyl acetate (200 mL). The organic layer was washed with water (100 mL) and brine (100 mL), and dried over anhydrous Na₂SO₄. The Na₂SO₄ was removed via filtration and the filtrate was concentrated under reduced pressure to afford the crude product which was triturated with diethyl ether and pet ether (1:1, 50 mL) to get desired 6-chloro-4-((3-(5-(difluoromethyl)-1,2,4-oxadiazol-3-yl)-2-methoxyphenyl)amino)-N-(methyl-d₃)pyridazine-3-carboxamide (500 mg, 0.834 mmol, 34.9% yield) as a brown solid. MS (M+1) m/z: 414.0 (M+H)⁺. LC retention time 2.72 min [F]. ¹H-NMR (400 MHz, DMSO-d6): δ 11.20 (s, 1H), 9.41 (s, 1H), 7.85-7.81 (m, 2H), 7.59 (t, J=51.20 Hz, 1H), 7.43 (d, J=8.00 Hz, 1H), 7.26 (s, 1H), 3.76 (s, 3H).

### Step 5

To a stirred solution of 6-chloro-4-((3-(5-(difluoromethyl)-1,2,4-oxadiazol-3-yl)-2-methoxyphenyl)amino)-N-(methyl-d₃)pyridazine-3-carboxamide (200 mg, 0.483 mmol) in 1,4-dioxane (5 mL) was added Cs₂CO₃ (472 mg, 1.450 mmol) and cyclopropanecarboxamide (82 mg, 0.967 mmol). The reaction mixture was purged with N₂ for 10 min and then 1,1-bis(di-cyclohexylphosphino)ferrocene (28.0 mg, 0.048 mmol) and Pd₂(dba)₃ (44.3 mg, 0.048 mmol) were added. The resultant reaction mixture was heated to 120° C. and stirred for 4 h in a sealed tube. The reaction mixture was filtered through a celite pad and washed with ethyl acetate (100 mL). The organic solvent was evaporated under reduced pressure and the resultant crude product was purified through reverse phase preparatory HPLC to obtain desired 6-(cyclopropanecarboxamido)-4-((3-(5-(difluoromethyl)-1,2,4-oxadiazol-3-yl)-2-methoxyphenyl)amino)-N-(methyl-d₃)pyridazine-3-carboxamide (25 mg, 0.051 mmol, 10.48% yield) as an off-white solid. MS (M+1) m/z: 463.2 (M+H)⁺. LC retention time 2.58 min [F]. ¹H-NMR (400 MHz, DMSO-D₆): δ 11.38 (s, 1H), 11.08 (s, 1H), 9.19 (s, 1H), 8.17 (s, 1H), 7.84 (s, 1H), 7.78-7.76 (m, 2H), 7.74-7.42 (m, 2H), 3.77 (s, 3H), 2.13-2.06 (m, 1H), 0.84 (t, J=2.00 Hz, 3H).

### Example 20

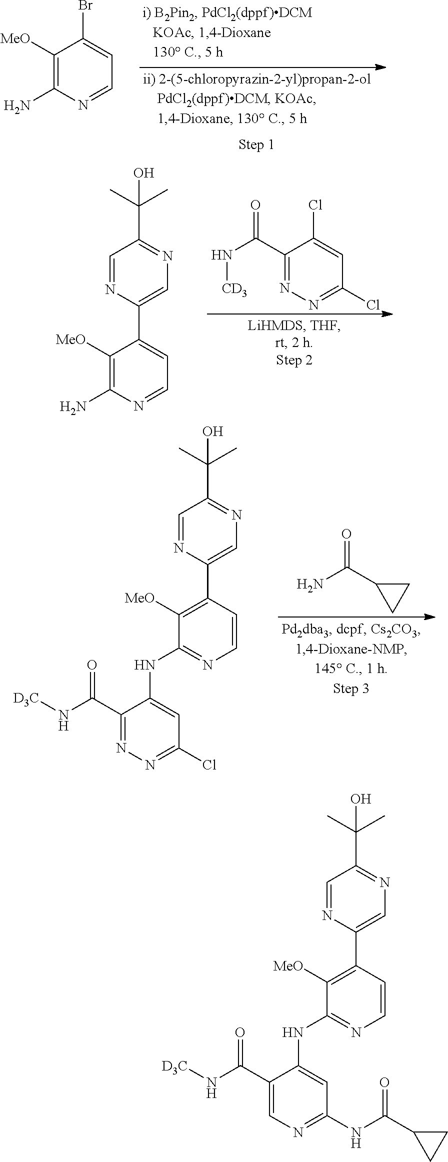 [SMILES: COc1c(Br)ccnc1N ; COc1c(-c2cnc(C(C)(C)O)cn2)ccnc1N ; [2H]C([2H])([2H])NC(=O)c1nnc(Cl)cc1Cl ; [2H]C([2H])([2H])NC(=O)c1nnc(Cl)cc1Nc1nccc(-c2cnc(C(C)(C)O)cn2)c1OC ; NC(=O)C1CC1 ; [2H]C([2H])([2H])NC(=O)c1cnc(NC(=O)C2CC2)cc1Nc1nccc(-c2cnc(C(C)(C)O)cn2)c1OC]

### Step 1

To a solution of 4-bromo-3-methoxypyridin-2-amine (370 mg, 1.822 mmol) and bis(pinacolato)diborane (972 mg, 3.83 mmol) in 1,4-dioxane (10 mL) was added potassium acetate (537 mg, 5.47 mmol) and the mixture purged with N₂ gas for 10 min. PdCl₂(dppf)·DCM (298 mg, 0.364 mmol) was added to the reaction and the mixture was heated to 130° C. and stirred for 5 h. The reaction mixture was filtered through a celite pad and washed with ethyl acetate (20 mL). The filtrate was concentrated under reduced pressure to afford crude product. The crude residue was dissolved in 1,4-dioxane (10 mL) and potassium acetate (537 mg, 5.47 mmol) was added at ambient temperature. The mixture was purged under N₂ gas for 10 min. PdCl₂(dppf)·DCM (298 mg, 0.364 mmol) and 2-(5-chloropyrazin-2-yl)propan-2-ol (315 mg, 1.822 mmol) were added and the mixture was heated to 130° C. and stirred for 5 h. The reaction mixture was diluted with ethyl acetate (50 mL) and filtered through a celite pad and washed with ethyl acetate (40 mL). The filtrate was concentrated under reduced pressure to afford crude product. The crude residue was purified by column chromatography using silica gel (100-200 mesh) with 100% of EtOAc as an eluent to obtain 2-(5-(2-amino-3-methoxypyridin-4-yl)pyrazin-2-yl)propan-2-ol (195 mg, 0.622 mmol, 34.1% yield) as a brown semisolid. MS (M+1) m/z: 260.7 (M+H)⁺. LC retention time 1.15 min [F].

### Step 2

To a stirred solution of 2-(5-(2-amino-3-methoxypyridin-4-yl)pyrazin-2-yl)propan-2-ol (195 mg, 0.749 mmol) and 4,6-dichloro-N-(methyl-d₃)pyridazine-3-carboxamide (157 mg, 0.749 mmol) in THE (10 mL) was added LiHMDS (2.24 mL, 2.24 mmol, 1 M solution in THF) at 0° C. The reaction mixture was stirred at rt for 1 h. The reaction was quenched via the addition of saturated aqueous NH₄Cl solution (20 mL). The product was extracted with ethyl acetate (50 mL). The organic layer was washed with water (20 mL) and brine (20 mL). The collected organic extract was dried over anhydrous Na₂SO₄, filtered and concentrated under reduced pressure. The crude product was triturated with diethylether (50 mL) and dried to obtain desired 6-chloro-4-((4-(5-(2-hydroxypropan-2-yl)pyrazin-2-yl)-3-methoxypyridin-2-yl)amino)-N-(methyl-d₃)pyridazine-3-carboxamide (100 mg, 22%) as a white solid. MS (M+1) m/z: 433.2 (M+H)⁺. LC retention time 2.42 min [F].

### Step 3

To a solution of 6-chloro-4-((4-(5-(2-hydroxypropan-2-yl)pyrazin-2-yl)-3-methoxypyridin-2-yl)amino)-N-(methyl-d₃)pyridazine-3-carboxamide (40 mg, 0.092 mmol) in 1,4-dioxane (2 mL) and NMP (0.2 mL) was added cyclopropanecarboxamide (15.73 mg, 0.185 mmol), xantphos (10.69 mg, 0.018 mmol), Cs₂CO₃ (75 mg, 0.231 mmol) and Pd₂dba₃ (16.92 mg, 0.018 mmol). The reaction mixture was stirred under microwave irradiation at 145° C. for 1 h. Reaction mixture was diluted with ethyl acetate (50 mL) and filtered through a celite pad and washed with ethyl acetate (40 mL). The filtrate was concentrated under reduced pressure to afford crude residue. The crude product was purified by reverse phase preparatory HPLC to obtain desired 6-(cyclopropanecarboxamido)-4-((4-(5-(2-hydroxypropan-2-yl)pyrazin-2-yl)-3-methoxypyridin-2-yl)amino)-N-(methyl-d₃)pyridazine-3-carboxamide (5.6 mg, 0.011 mmol, 12.37% yield) as an off-white solid. MS (M+1) m/z: 482.2 (M+H)⁺. LC retention time 1.89 min [F]. ¹H-NMR (400 MHz, DMSO-d₆): δ 12.47 (s, 1H), 11.38 (s, 1H), 9.89 (s, 1H), 9.28 (s, 1H), 9.11 (dd, J=1.60, 17.20 Hz, 2H), 8.23 (d, J=5.20 Hz, 1H), 7.43 (d, J=5.20 Hz, 1H), 5.60 (s, 1H), 3.69 (s, 3H), 2.25-2.10 (m, 1H), 1.54 (s, 6H), 0.91-0.88 (m, 4H).

The following examples were prepared from described intermediates along with commercial reagents using conditions referred to in the table (described below) allowing for some minor modifications to solvent, reaction time, etc.

| | | Headpiece | SnAr | m/z | Rt (min) |
|---|---|---|---|---|---|
| Ex. | Structure | sequence | conditions | [M +H]⁺ | [Method] |
| 21 | 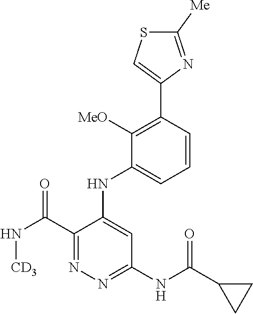 [SMILES: [2H]C([2H])([2H])NC(=O)c1nnc(NC(=O)C2CC2)cc1Nc1cccc(-c2csc(C)n2)c1OC] | α | C | 442.2 | 1.69 [B] |
| 22 | 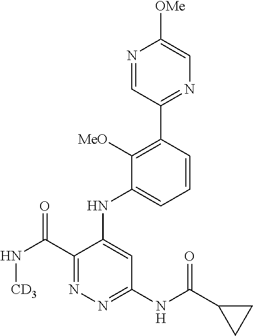 [SMILES: [2H]C([2H])([2H])NC(=O)c1nnc(NC(=O)C2CC2)cc1Nc1cccc(-c2cnc(OC)cn2)c1OC] | α | C | 453.3 | 1.70 [B] |
| 23 | 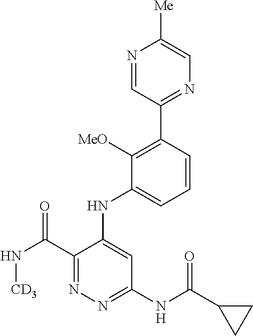 [SMILES: [2H]C([2H])([2H])NC(=O)c1nnc(NC(=O)C2CC2)cc1Nc1cccc(-c2cnc(C)cn2)c1OC] | α | C | 437.0 | 0.73 [D] |
| 24 | 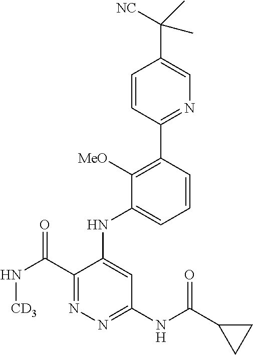 [SMILES: [2H]C([2H])([2H])NC(=O)c1nnc(NC(=O)C2CC2)cc1Nc1cccc(-c2ccc(C(C)(C)C#N)cn2)c1OC] | α | C | 489.2 | 1.71 [B] |
| 25 | 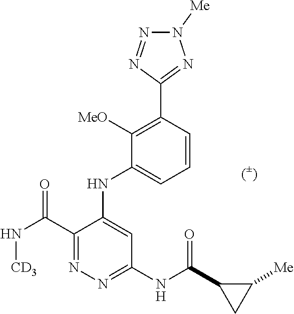 | (*) | C | 441.4 | 0.78 [D] |
| 26 | 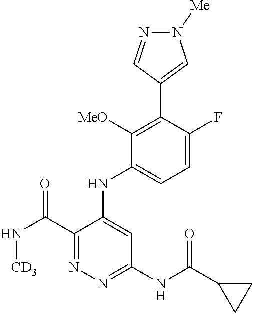 [SMILES: [2H]C([2H])([2H])NC(=O)c1nnc(NC(=O)C2CC2)cc1Nc1ccc(F)c(-c2cnn(C)c2)c1OC] | β | C | 443.2 | 1.54 [B] |
| 27 | 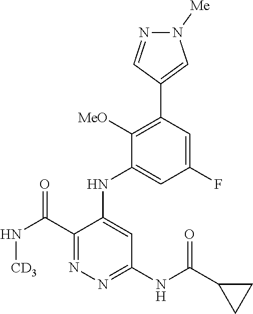 [SMILES: [2H]C([2H])([2H])NC(=O)c1nnc(NC(=O)C2CC2)cc1Nc1cc(F)cc(-c2cnn(C)c2)c1OC] | β | C | 443.1 | 1.65 [B] |
| 28 | 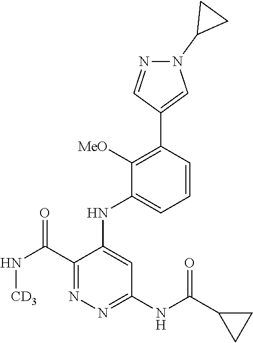 [SMILES: [2H]C([2H])([2H])NC(=O)c1nnc(NC(=O)C2CC2)cc1Nc1cccc(-c2cnn(C3CC3)c2)c1OC] | β | C | 451.1 | 1.72 [B] |
| 29 | 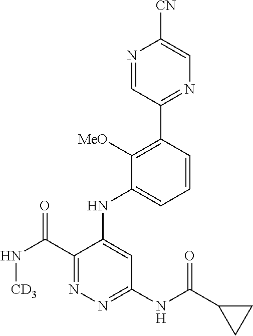 [SMILES: [2H]C([2H])([2H])NC(=O)c1nnc(NC(=O)C2CC2)cc1Nc1cccc(-c2cnc(C#N)cn2)c1OC] | α | C | 448.1 | 1.76 [B] |
| 30 | 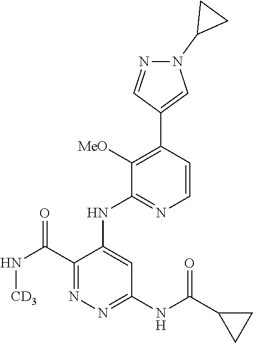 [SMILES: [2H]C([2H])([2H])NC(=O)c1nnc(NC(=O)C2CC2)cc1Nc1nccc(-c2cnn(C3CC3)c2)c1OC] | β | B | 452.1 | 1.65 [B] |
| 31 | 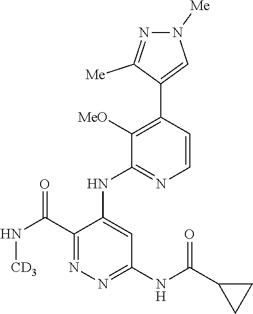 [SMILES: [2H]C([2H])([2H])NC(=O)c1nnc(NC(=O)C2CC2)cc1Nc1nccc(-c2cn(C)nc2C)c1OC] | β | B | 440.2 | 1.55 [B] |
| 32 | 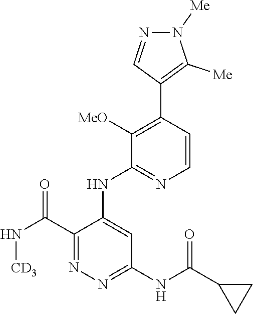 [SMILES: [2H]C([2H])([2H])NC(=O)c1nnc(NC(=O)C2CC2)cc1Nc1nccc(-c2cnn(C)c2C)c1OC] | β | B | 440.2 | 1.51 [B] |
| 33 | 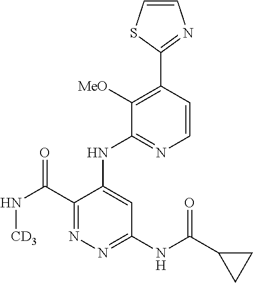 [SMILES: [2H]C([2H])([2H])NC(=O)c1nnc(NC(=O)C2CC2)cc1Nc1nccc(-c2nccs2)c1OC] | γ | A | 429.0 | 1.70 [B] |
| 34 | 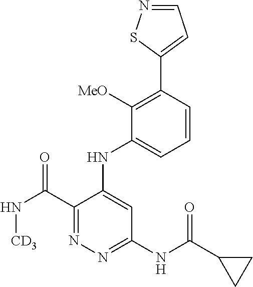 [SMILES: [2H]C([2H])([2H])NC(=O)c1nnc(NC(=O)C2CC2)cc1Nc1cccc(-c2ccns2)c1OC] | α | C | 428.2 | 1.71 [B] |
| 35 | 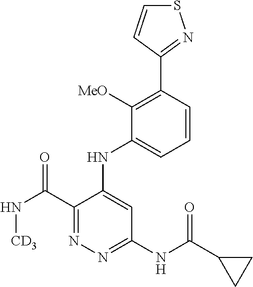 [SMILES: [2H]C([2H])([2H])NC(=O)c1nnc(NC(=O)C2CC2)cc1Nc1cccc(-c2ccsn2)c1OC] | α | C | 428.2 | 1.78 [B] |
| 36 | 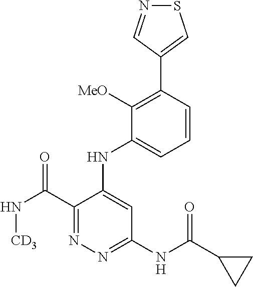 [SMILES: [2H]C([2H])([2H])NC(=O)c1nnc(NC(=O)C2CC2)cc1Nc1cccc(-c2cnsc2)c1OC] | β | C | 428.2 | 1.72 [B] |
| 37 | 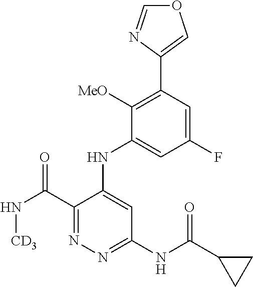 [SMILES: [2H]C([2H])([2H])NC(=O)c1nnc(NC(=O)C2CC2)cc1Nc1cc(F)cc(-c2cocn2)c1OC] | α | C | 430.2 | 1.72 [B] |
| 38 | 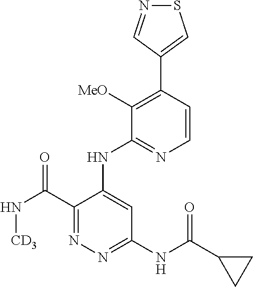 [SMILES: [2H]C([2H])([2H])NC(=O)c1nnc(NC(=O)C2CC2)cc1Nc1nccc(-c2cnsc2)c1OC] | β | B | 429.1 | 1.67 [B] |
| 39 | 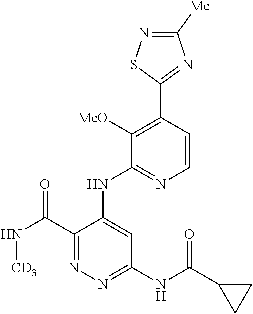 [SMILES: [2H]C([2H])([2H])NC(=O)c1nnc(NC(=O)C2CC2)cc1Nc1nccc(-c2nc(C)ns2)c1OC] | γ | A | 444.2 | 1.83 [B] |
| 40 | 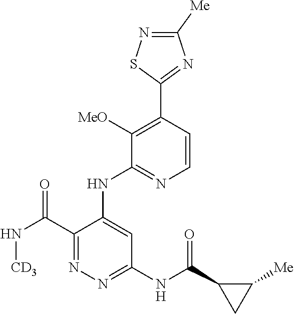 [SMILES: [2H]C([2H])([2H])NC(=O)c1nnc(NC(=O)[C@@H]2C[C@H]2C)cc1Nc1nccc(-c2nc(C)ns2)c1OC] | γ | A | 458.2 | 1.98 [B] |
| 41 | 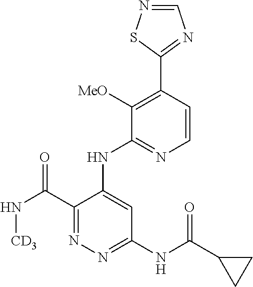 [SMILES: [2H]C([2H])([2H])NC(=O)c1nnc(NC(=O)C2CC2)cc1Nc1nccc(-c2ncns2)c1OC] | γ | A | 430.1 | 1.65 [B] |
| 42 | 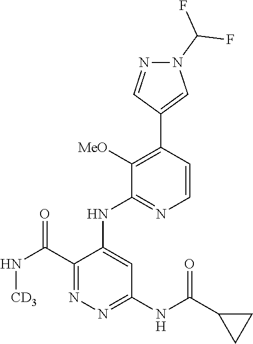 [SMILES: [2H]C([2H])([2H])NC(=O)c1nnc(NC(=O)C2CC2)cc1Nc1nccc(-c2cnn(C(F)F)c2)c1OC] | β | A | 462.3 | 1.61 [I] |

(*)—used the procedure from U.S. Patent RE47929

### Headpiece Sequence α

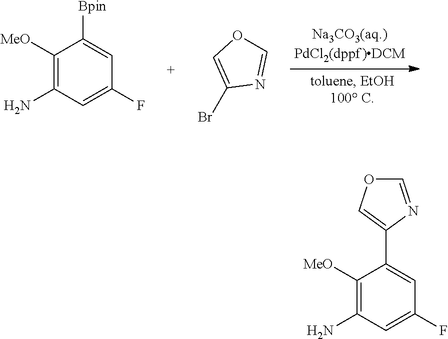

A solution of 4-bromooxazole, HCl (78 mg, 0.424 mmol), 5-fluoro-2-methoxy-3-(4,4,5,5-tetramethyl-1,3,2-dioxaborolan-2-yl)aniline (103 mg, 0.386 mmol), and PdCl₂(dppf)·DCM (15.75 mg, 0.019 mmol) in toluene (3 ml) was degassed by bubbling nitrogen through the reaction solution for 5 minutes, then sodium carbonate (10% aq. solution) (1.23 ml, 1.16 mmol) was added and the resulting mixture was stirred at 100° C. for 2 h. The two layers were partitioned, the organic layer was collected and then purified via automated chromatography eluting with 0-100% EtOAc in hexanes. Afforded 5-fluoro-2-methoxy-3-(oxazol-4-yl)aniline (17 mg, 0.082 mmol, 21.18% yield), a light yellow oil which appears to be quite volatile under vacuum. m/z (M+H)=209.1. RT=1.06 min [C]

### Headpiece Sequence β

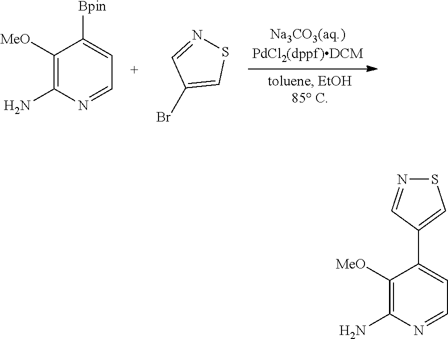

A mixture of 4-(4,4,5,5-tetramethyl-1,3,2-dioxaborolan-2-yl)isothiazole (135 mg, 0.640 mmol), 4-bromo-3-methoxypyridin-2-amine (100 mg, 0.493 mmol), and PdCl₂(dppf)·DCM (20.11 mg, 0.025 mmol) in toluene (3 m) and ethanol (1 mL) was degassed by bubbling nitrogen through the reaction solution for 5 minutes. Sodium carbonate (10% aq. solution) (1.8 mL, 1.7 mmol) was added, the vessel was sealed, and the reaction was stirred at 85° C. for 4 h. The two layers of the biphasic reaction mixture were separated, and the organic layer was concentrated to an oil and purified by automated chromatography eluting with 0-100% EtOAc in hexanes. Afforded 4-(isothiazol-4-yl)-3-methoxypyridin-2-amine (58 mg, 0.280 mmol, 56.8% yield) as an off white waxy solid. m/z (M+H)=208.1. RT=0.62 min [C]

### Headpiece Sequence γ

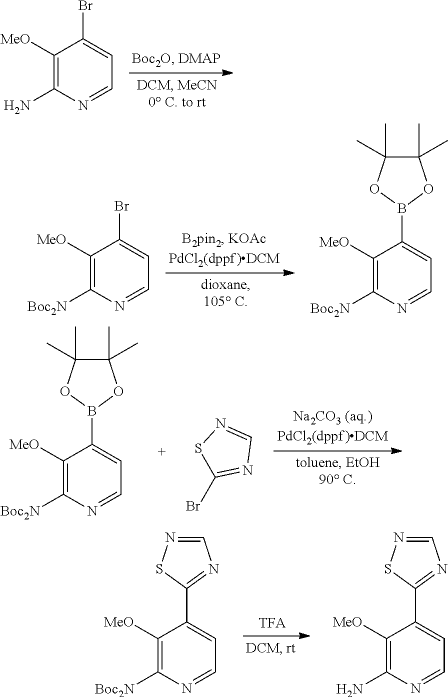

### Step 1

DMAP (0.277 g, 2.266 mmol) and BOC-anhydride (3.29 mL, 14.16 mmol) in DCM (24 mL) were added to a solution of 4-bromo-3-methoxypyridin-2-amine (1.15 g, 5.66 mmol) in acetonitrile (24 mL) at 0° C. The resulting mixture was allowed to warm to room temperature and, after an hour, an additional 200 mg of BOC-anhydride was added in 2 mL DCM and the reaction was stirred overnight. The crude reaction was concentrated under reduced pressure and purified via automated chromatography eluting with 0-50% EtOAc in hexanes. Pure fractions were combined and concentrated to provide tert-butyl (4-bromo-3-methoxypyridin-2-yl)(tert-butoxycarbonyl)carbamate (2.29 g, 5.39 mmol, 95% yield) as a crystalline off-white solid. m/z (M+H)=404.9. RT=1.66 min [C].

### Step 2

A solution of tert-butyl (4-bromo-3-methoxypyridin-2-yl)(tert-butoxycarbonyl)carbamate (800 mg, 1.984 mmol), 4,4,4′,4′,5,5,5′,5′-octamethyl-2,2′-bi(1,3,2-dioxaborolane) (655 mg, 2.58 mmol), PdCl₂(dppf)·DCM (81 mg, 0.099 mmol) and potassium acetate (681 mg, 6.94 mmol) in dioxane (8 mL) in a pressure vial was heated to reflux. Upon completion of the reaction, as determined via LCMS, the reaction was absorbed directly onto celite and the celite was dried under reduce pressure. The reaction was then purified via automated chromatography (dry loading), eluting with 0-100% EtOAc in hexanes. Afforded tert-butyl (tert-butoxycarbonyl)(3-methoxy-4-(4,4,5,5-tetramethyl-1,3,2-dioxaborolan-2-yl)pyridin-2-yl)carbamate (575 mg, 1.28 mmol, 64.4% yield) as a colorless oil. m/z (M+H)=369.0 (observed as boronic acid—formed in MS). RT=1.26 min [C]. ¹H NMR (400 MHz, DMSO-d₆) δ 9.15 (s, 1H), 8.49 (d, J=5.1 Hz, 1H), 8.21 (d, J=5.0 Hz, 1H), 4.01 (s, 3H), 1.39 (s, 18H).

### Step 3

A stirred mixture of 5-bromo-1,2,4-thiadiazole (274 mg, 1.660 mmol), tert-butyl (tert-butoxycarbonyl)(3-methoxy-4-(4,4,5,5-tetramethyl-1,3,2-dioxaborolan-2-yl)pyridin-2-yl)carbamate (575 mg, 1.28 mmol) and sodium carbonate (10% aq. solution) (4.06 mL, 3.83 mmol) in toluene (4 mL) and ethanol (2 mL) was degassed by bubbling nitrogen through the mixture for 5 minutes. PdCl₂(dppf)·DCM (52.1 mg, 0.064 mmol) was quickly added and the reaction mixture heated at 90° C. for 40 minutes. The reaction mixture was cooled to room temperature then diluted with EtOAc (75 mL). This solution was dried over sodium sulfate, filtered, concentrated under reduced pressure. The crude product was purified via automated chromatography, eluting with 0-100% EtOAc in hexanes. Afforded tert-butyl (tert-butoxycarbonyl)(3-methoxy-4-(1,2,4-thiadiazol-5-yl)pyridin-2-yl)carbamate (279 mg, 0.683 mmol, 53.5% yield) as an off-white solid. m/z (M+H)=409.2. RT=1.62 min [C]. ¹H NMR (400 MHz, DMSO-d₆) δ 9.15 (s, 1H), 8.49 (d, J=5.1 Hz, 1H), 8.21 (d, J=5.0 Hz, 1H), 4.01 (s, 3H), 1.39 (s, 18H).

### Step 4

A solution of tert-butyl (tert-butoxycarbonyl)(3-methoxy-4-(1,2,4-thiadiazol-5-yl)pyridin-2-yl)carbamate (278 mg, 0.681 mmol) in DCM (2 mL) and TFA (1 mL) was stirred at room temperature for 30 minutes. The crude reaction was concentrated under reduced pressure to afford a white solid. To this was added EtOAc and a 1.5 M (aq.) potassium phosphate solution. The layers were separated and the aq. layer was extracted twice with EtOAc. The organic layers were combined and washed once with brine. The organic layer was then dried over sodium sulfate, filtered and concentrated under reduced pressure to afford 3-methoxy-4-(1,2,4-thiadiazol-5-yl)pyridin-2-amine (130 mg, 0.624 mmol, 92% yield) as the free base, a light yellow solid. m/z (M+H)=209.0. RT=0.65 min [C].

### SnAr Conditions A:

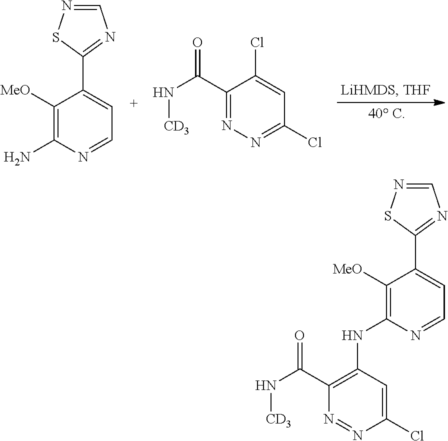 [SMILES: COc1c(-c2ncns2)ccnc1N ; [2H]C([2H])([2H])NC(=O)c1nnc(Cl)cc1Cl ; [2H]C([2H])([2H])NC(=O)c1nnc(Cl)cc1Nc1nccc(-c2ncns2)c1OC]

To a solution of 3-methoxy-4-(1,2,4-thiadiazol-5-yl)pyridin-2-amine (110 mg, 0.528 mmol) and 4,6-dichloro-N-(methyl-d₃)pyridazine-3-carboxamide (221 mg, 1.056 mmol) in THE (4 mL) at rt was added, dropwise over 5 minutes, LiHMDS (1M in THF, 2.38 mL, 2.38 mmol). The reaction mixture was allowed to stir at rt for 30 minutes. After quenching with saturated ammonium chloride solution, the THF was removed under reduced pressure and the reaction mixture was diluted with water. Filtration, washing the filter cake with water and ether and drying afforded a brown solid that was suspended in ether. After sonication the suspension was filtered and dried to afford 6-chloro-4-((3-methoxy-4-(1,2,4-thiadiazol-5-yl)pyridin-2-yl)amino)-N-(methyl-d₃)pyridazine-3-carboxamide (171 mg, 0.404 mmol, 77% yield) as a tan solid. m/z (M+H)=381.0. RT=1.49 min [C]

### SnAr Conditions B:

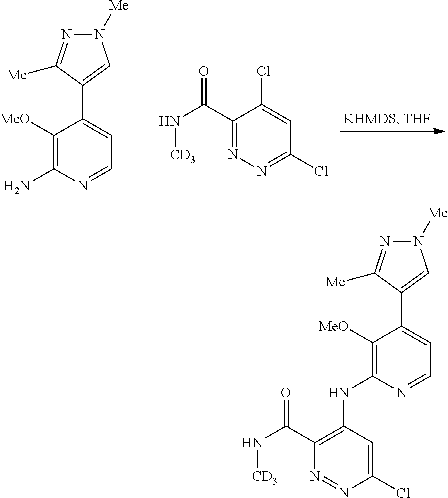 [SMILES: COc1c(-c2cn(C)nc2C)ccnc1N ; [2H]C([2H])([2H])NC(=O)c1nnc(Cl)cc1Cl ; [2H]C([2H])([2H])NC(=O)c1nnc(Cl)cc1Nc1nccc(-c2cn(C)nc2C)c1OC]

To a solution of 4,6-dichloro-N-(methyl-d₃)pyridazine-3-carboxamide (67.4 mg, 0.323 mmol) and 4-(1,3-dimethyl-1H-pyrazol-4-yl)-3-methoxypyridin-2-amine (44 mg, 0.202 mmol) in tetrahydrofuran (2 mL) was added potassium bis(trimethylsilyl)amide (0.907 mL, 0.907 mmol) in a dropwise manner and reaction stirred until complete (˜15 min). A solution of saturated ammonium chloride (aq.) was added to quench the residual base. Then the reaction was partitioned between EtOAc and water. The water layer was extracted once with EtOAc, and then the combined organic layers were washed once with a saturated aqueous ammonium chloride solution and once with brine. The combined organic layers were dried over sodium sulfate, filtered and concentrated under reduced pressure. The crude product was purified using automated chromatography eluting with 0-100% EtOAc in hexanes. Afforded 6-chloro-4-((4-(1,3-dimethyl-1H-pyrazol-4-yl)-3-methoxypyridin-2-yl)amino)-N-(methyl-d₃)pyridazine-3-carboxamide (27 mg, 0.069 mmol, 34.3% yield) as a pale yellow solid. m/z (M+H)=391.1. RT=1.30 min [C]

### SnAr Conditions C:

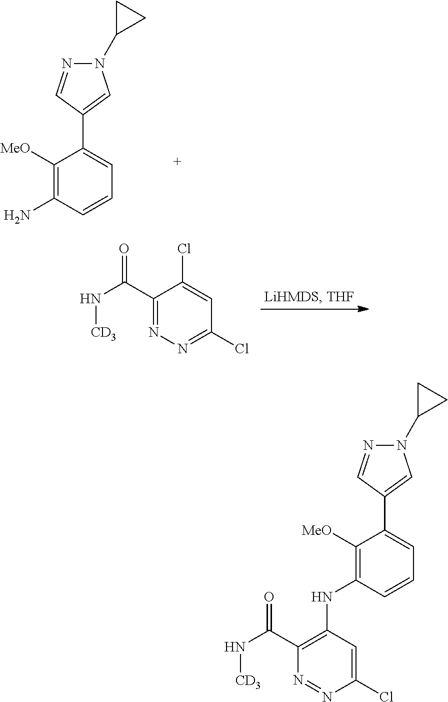 [SMILES: COc1c(N)cccc1-c1cnn(C2CC2)c1 ; [2H]C([2H])([2H])NC(=O)c1nnc(Cl)cc1Cl ; [2H]C([2H])([2H])NC(=O)c1nnc(Cl)cc1Nc1cccc(-c2cnn(C3CC3)c2)c1OC]

To a solution of 4,6-dichloro-N-(methyl-d₃)pyridazine-3-carboxamide (55 mg, 0.263 mmol) and 3-(1-cyclopropyl-1H-pyrazol-4-yl)-2-methoxyaniline (60.3 mg, 0.263 mmol) in tetrahydrofuran (2 mL) was added 1M lithium bis(trimethylsilyl)amide (0.658 mL, 0.658 mmol) in a dropwise manner (<5 min) using a syringe and the reaction stirred until complete (˜15 min). Saturated aqueous ammonium chloride was added to quench the residual base. The reaction was partitioned between EtOAc and water. The water layer was washed once with EtOAc, and the combined organic layers were washed once with saturated aqueous ammonium chloride solution and once with brine solution. The combined organic layers were then dried over sodium sulfate, filtered and concentrated under reduced pressure to provide 6-chloro-4-((3-(1-cyclopropyl-1H-pyrazol-4-yl)-2-methoxyphenyl)amino)-N-(methyl-d₃)pyridazine-3-carboxamide (69 mg, 0.172 mmol, 65.3% yield). m/z (M+H)=402.1, RT=1.39 min [C].

### Example 43

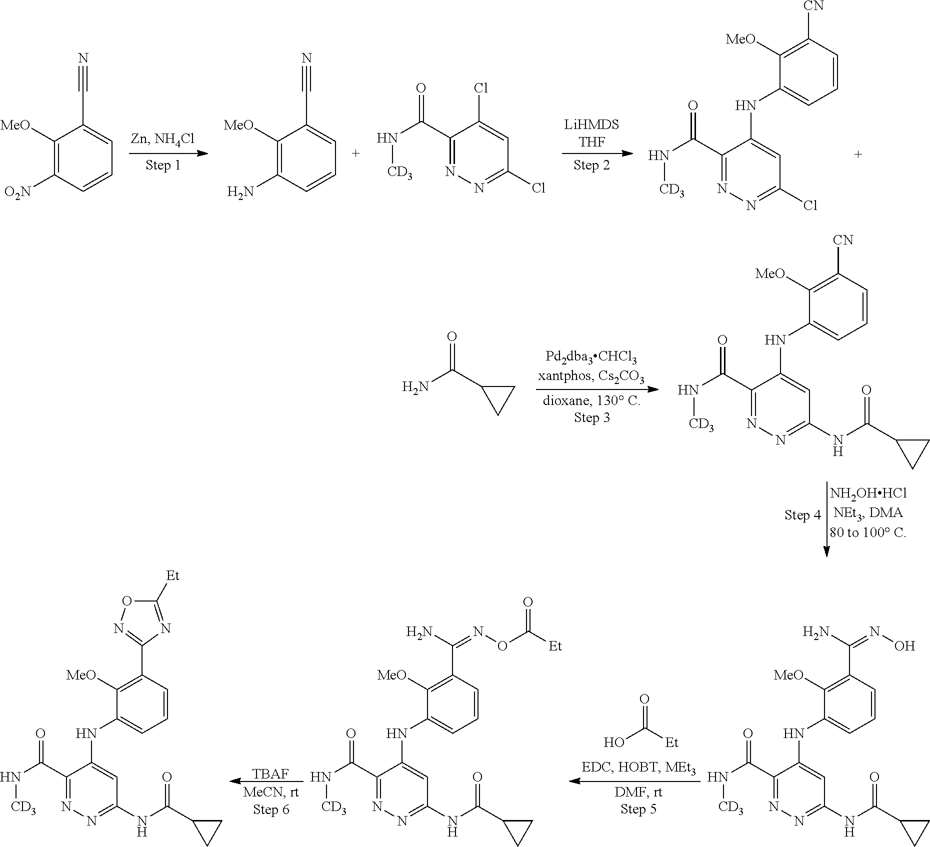

### Step 1

To a stirred solution of 2-methoxy-3-nitrobenzonitrile (4.0 g, 22.23 mmol) in THF: MeOH (1:1, 80 mL) was added zinc powder (14.7 g, 230.2 mmol) followed by ammonium chloride (6.0 g, 112.1 mmol) at room temperature. After stirring for 4 h at room temperature, the reaction mixture was filtered through a celite pad and washed with ethyl acetate (2×50 mL). The filtrate was concentrated under reduced pressure. Trituration of the crude product in diethyl ether (50 mL) yielded 3-amino-2-methoxybenzonitrile (3.3 g, 7.69 mmol, 97% yield) as off white solid.

¹H NMR (DMSO-d6): 6.97-6.96 (s, 2H), 6.85-6.82 (m, 1H), 5.45 (s, 2H), 3.80 (s, 3H).

### Step 2

To a stirred solution of 4,6-dichloro-N-trideutero-methylpyriazine-3-carboxamide (325 mg, 1.56 mmol) and 3-amino-2-methoxybenzonitrile (255 mg, 1.72 mmol) in THE (14 mL) was added LiHMDS (1M in THF, 3.9 mL, 3.9 mmol) in a dropwise manner. The resulting solution was stirred at room temperature for 1 h and then quenched via the addition of saturated (aq.) ammonium chloride (2 mL). The mixture was partitioned between EtOAc (40 ml) and saturated ammonium chloride solution (40 ml). the organic layer was washed with brine (40 ml), dried (Na₂SO₄) and concentrated to afford a solid residue that was purified using automated chromatography, eluting with a 0-100% EtOAc/Hex gradient. The pure fractions were concentrated to afford a partially purified product that was triturated with ether and dried to afford 6-chloro-4-((3-cyano-2-methoxyphenyl)amino)-N-trideutero-methylpyridazine-3-carboxamide (385 mg, 1.200 mmol, 77% yield) as a tan solid. ¹H NMR (400 MHz, DMSO-d₆) δ 11.10 (s, 1H), 9.39 (br. s., 1H), 7.87 (d, J=7.9 Hz, 1H), 7.67 (d, J=7.7 Hz, 1H), 7.35 (t, J=7.9 Hz, 1H), 7.22 (s, 1H), 3.91 (s, 3H). LC retention time=0.82 min [D]. m/z 321.2 (MH⁺).

### Step 3

A mixture of 6-chloro-4-((3-cyano-2-methoxyphenyl)amino)-N-trideutero-methylpyridazine-3-carboxamide (240 mg, 0.748 mmol), cyclopropanecarboxamide (127 mg, 1.496 mmol), Pd₂(dba)₃, chloroform adduct (77 mg, 0.075 mmol), xantphos (87 mg, 0.150 mmol) and Cs₂CO₃ (975 mg, 2.99 mmol) in dioxane (5 mL) was degassed by bubbling N2 through the mixture for 5 minutes. The reaction vessel was sealed and heated to 130° C. for 1.5 hr. The reaction mixture was filtered hot (˜90° C.) through celite and the filter cake was washed with EtOAc (100 mL). The filtrate was concentrated and the residue was triturated with MeOH. Filtration and drying afforded 4-((3-cyano-2-methoxyphenyl)amino)-6-(cyclopropanecarboxamido)-N-trideutero-methylpyridazine-3-carboxamide (215 mg, 0.582 mmol, 78% yield) as a tan solid. LC retention time=0.74 min [D]. m/z 370.4 (MH⁺).

### Step 4

A mixture of 4-((3-cyano-2-methoxyphenyl)amino)-6-(cyclopropanecarboxamido)-N-trideutero-methylpyridazine-3-carboxamide (215 mg, 0.582 mmol), hydroxylamine hydrochloride (121 mg, 1.746 mmol) and triethylamine (0.406 mL, 2.91 mmol) in DMA (5 mL) was heated to 80° C. for 2 weeks—conversion incomplete. Additional hydroxylamine hydrochloride (121 mg, 1.746 mmol) and triethylamine (0.406 mL, 2.91 mmol) were added and the reaction mixture was heated to 100° C. for 16 hr. After cooling to rt, the reaction mixture was partitioned between EtOAc (50 mL) and 10% (aq.) LiCl solution (50 mL). The organic layer was washed with 10% (aq.) LiCl solution (2×50 mL) and brine (50 mL). After drying (Na₂SO₄) and filtration the organic layer was concentrated to a yellow oil that was purified using automated chromatography to afford (Z)-6-(cyclopropanecarboxamido)-4-((3-(N′-hydroxycarbamimidoyl)-2-methoxyphenyl)amino)-N-trideutero-methylpyridazine-3-carboxamide (78 mg, 0.194 mmol, 33.3% yield) as a tan solid. LC retention time=0.51 min [D]. m/z 403.3 (MH⁺).

### Step 5

A mixture of (Z)-6-(cyclopropanecarboxamido)-4-((3-(N′-hydroxycarbamimidoyl)-2-methoxyphenyl)amino)-N-trideutero-methylpyridazine-3-carboxamide (25 mg, 0.062 mmol), propionic acid (9.30 μL, 0.124 mmol), HOBT (10.46 mg, 0.068 mmol), EDC (13.10 mg, 0.068 mmol) and triethylamine (0.035 mL, 0.248 mmol) was stirred at rt for 5 hr. The reaction mixture was partitioned between EtOAc (20 mL) and saturated (aq.) NaHCO₃ (20 mL). The organic layer was washed with 10% (aq.) LiCl solution (2×20 mL) and brine (20 mL). After drying (Na₂SO₄) and filtration the organic layer was concentrated to afford (Z)-6-(cyclopropanecarboxamido)-4-((2-methoxy-3-(N′-(propionyloxy)carbamimidoyl)phenyl)amino)-N-trideuteromethylpyridazine-3-carboxamide (27 mg, 0.059 mmol, 95% yield) as an off-white solid. LC retention time=0.76 min [D]. m/z 459.5 (MH⁺).

### Step 6

A mixture of (Z)-6-(cyclopropanecarboxamido)-4-((2-methoxy-3-(N′-(propionyloxy)carbamimidoyl)phenyl)amino)-N-trideuteromethylpyridazine-3-carboxamide (27 mg, 0.059 mmol) and TBAF 1M in THE (0.088 mL, 0.088 mmol) in acetonitrile (1 mL) was allowed to stir at rt for 3 hr. The solvent was removed in vacuo and the residue was dissolved in DMSO and purified by preparative HPLC to yield the desired product (13.8 mg, 50% yield). LC retention time=1.38 min [B]. m/z 441.3 (MH⁺). ¹H NMR (500 MHz, DMSO-d₆) δ 11.34 (s, 1H), 11.01 (s, 1H), 9.15 (s, 1H), 8.13 (s, 1H), 7.68 (dd, J=18.0, 7.9 Hz, 2H), 7.37 (t, J=7.9 Hz, 1H), 3.72 (s, 3H), 3.02 (q, J=7.4 Hz, 2H), 2.06 (br. s., 1H), 1.35 (t, J=7.6 Hz, 3H), 0.87-0.72 (m, 4H).

| Example No. | ¹H NMR |
|---|---|
| 3 | ¹H-NMR (400 MHz, DMSO-d₆): δ 11.37 (s, |
| | 1H), 11.05 (s, 1H), 9.18 (s, 1H), 8.15 (s, 1H), |
| | 7.74-7.69 (m, 2H), 7.42-7.38 (m, 1H), 4.89 |
| | (s, 2H), 3.75 (s, 3H), 3.69-3.64 (m, 2H), |
| | 2.12-2.06 (m, 1H), 1.21-1.18 |
| | (m, 3H), 0.84-0.82 (m, 4H). |
| 4 | ¹H-NMR (400 MHz, DMSO-d₆): δ |
| | 11.36 (s, 1H), 11.04 (s, 1H), 9.17 |
| | (s, 1H), 8.15 (s, 1H), 7.72-7.67 |
| | (m, 2H), 7.41-7.37 (m, 1H), 3.83- |
| | 3.80 (m, 2H), 3.75 (s, 3H), 3.31-3.27 |
| | (m, 5H), 2.12-2.06 (m, 1H), |
| | 0.84-0.82 (m, 4H). |
| 8 | ¹H-NMR (400 MHz, DMSO-d₆): δ |
| | 11.35 (s, 1H), 11.02 (s, 1H), 9.16 |
| | (s, 2H), 8.15 (s, 1H), 7.87 (s, 1H), |
| | 7.61 (m, 2H), 7.36 (m, 1H), 3.54 |
| | (s, 3H), 2.54 (s, 3H), 2.08-2.11 |
| | (m, 1H), 0.83-0.84 (m, 4H). |
| 9 | ¹H-NMR (400 MHz, DMSO-d₆): δ |
| | 11.37 (s, 1H), 10.91 (s, 1H), 9.21 |
| | (s, 1H), 9.00 (s, 1H), 8.15-8.17 |
| | (m, 1H), 8.03 (m, 1H), 7.69-7.71 (m, |
| | 1H), 7.44-7.47 (m, 1H), 3.90 (s, 3H), |
| | 2.04-2.11 (m, 1H), 0.78-0.82 |
| | (m, 4H). |
| 10 | ¹H-NMR (400 MHz, DMSO-d₆): δ |
| | 11.35 (s, 1H), 11.01 (s, 1H), 9.14 |
| | (s, 1H), 8.84 (d, J = 8.4 Hz, 1H), |
| | 8.76-8.76 (m, 2H), 7.59 (d, J = 8.4 |
| | Hz, 1H), 7.56 (d, J = 7.6 Hz, 1H), |
| | 7.37 (d, J = 15.6 Hz, 1H), 3.53 (s, |
| | 3H), 2.07-2.10 (m, 1H), 0.83-0.85 (m, 4H). |
| 11 | ¹H-NMR (400 MHz, DMSO-d₆): δ |
| | 11.33 (s, 1H), 10.97 (s, 1H), |
| | 9.15 (s, 1H), 8.88 (d, J = 1.6 Hz, 1H), |
| | 8.75 (d, J = 1.2 Hz, 1H), 8.17 |
| | (s, 1H), 7.56 (d, J = 1.6 Hz, 1H), 7.54 |
| | (d, J = 2.4 Hz, 1H), 7.35 (d, J = |
| | 8.0 Hz, 1H), 3.52 (s, 3H), 2.27-2.25 |
| | (m, 1H), 2.08-2.10 (m, 1H), |
| | 1.12-1.10 (m, 2H), 1.09-1.10 |
| | (m, 2H), 1.08-1.05 (m, 4H). |
| 12 | ¹H-NMR (400 MHz, DMSO-d₆): 11.38 |
| | (s, 1H), 11.05 (s, 1H), 9.67 |
| | (d, J = 1.2 Hz, 1H), 9.19 (d, J = 4.8 |
| | Hz, 2H), 8.17 (s, 1H), 7.69 (d, J = |
| | 8.0 Hz, 2H), 7.42-7.46 (m, 1H), 3.58 |
| | (s, 3H), 2.04-2.10 (m, 1H), |
| | 0.85-0.83 (m, 4H). |
| 13 | ¹H-NMR (400 MHz, DMSO-d₆): δ |
| | 11.37 (s, 1H), 10.97 (s, 1H), 9.21 |
| | (s, 1H), 8.10-8.12 (m, 1H), 8.03 |
| | (s, 1H), 7.67-7.69 (m, 1H), 7.41- |
| | 7.45 (m, 1H), 3.87 (s, 3H), 2.67 |
| | (s, 3H), 2.06-2.09 (m, 1H), 0.79- |
| | 0.85 (m, 4H). |
| 15 | ¹H-NMR (400 MHz, DMSO-d₆): δ |
| | 11.45 (s, 1H), 11.18 (s, 1H), 9.22 |
| | (s, 1H), 9.09 (d, J = 1.20 Hz, 1H), |
| | 8.94 (d, J = 1.60 Hz, 1H), 8.28 (s, |
| | 1H), 7.50 (dd, J = 2.80, 9.40 Hz, 1H), |
| | 7.39 (dd, J = 3.20, 9.20 Hz, |
| | 1H), 6.10 (s, 1H), 3.55 (s, 3H), |
| | 2.68-2.50 (m, 2H), 2.33-2.27 (m, |
| | 2H), 2.17-2.10 (m, 1H), 1.99-1.92 |
| | (m, 2H), 0.87-0.85 (m, 4H). |
| 18 | ¹H-NMR (400 MHz, DMSO-d₆): δ |
| | 11.33 (s, 1H), 10.95 (s, 1H), 9.14 |
| | (s, 1H), 8.15 (s, 1H), 7.45-7.43 (m, |
| | 1H), 7.30-7.23 (m, 2H), 3.47 (s, |
| | 3H), 2.42 (s, 3H), 2.29 (s, 3H), |
| | 2.09-2.07 (m, 1H), 0.83-0.81 (m, 4H). |
| 21 | ¹H NMR (500 MHz, DMSO-d₆) δ |
| | 11.31 (s, 1H), 10.97 (s, 1H), 9.13 |
| | (s, 1H), 8.14 (s, 1H), 7.96 (s, 1H), |
| | 7.87 (d, J = 7.7 Hz, 1H), 7.42 (d, |
| | J = 7.7 Hz, 1H), 7.26 (t, J = 7.9 Hz, 1H), |
| | 3.62 (s, 3H), 2.72 (s, 3H), 2.13- |
| | 1.94 (m, 1H), 0.89-0.77 (m, 4H). |
| 22 | ¹H NMR (500 MHz, DMSO-d₆) δ |
| | 11.32 (s, 1H), 10.97 (s, 1H), 9.14 |
| | (s, 1H), 8.66 (s, 1H), 8.44 (s, 1H), |
| | 8.16 (s, 1H), 7.52 (dd, J = 12.3, 7.9 |
| | Hz, 2H), 7.37-7.26 (m, 1H), 3.98 |
| | (s, 3H), 3.52 (s, 3H), 2.14-2.00 |
| | (m, 1H), 0.89-0.74 (m, 4H). |
| 23 | ¹H NMR (400 MHz, DMSO-d₆) δ |
| | 11.33 (s, 1H), 10.99 (s, 1H), 9.14 |
| | (s, 1H), 8.94 (d, J = 1.5 Hz, 1H), 8.67 |
| | (d, J = 1.0 Hz, 1H), 8.17 (s, 1H), |
| | 7.55 (d, J = 8.1 Hz, 2H), 7.42-7.26 |
| | (m, 1H), 3.51 (s, 3H), 3.31 (s, 3H), |
| | 2.17-2.00 (m, 1H), 0.92-0.72 (m, 4H). |
| 24 | ¹H NMR (500 MHz, DMSO-d₆) δ |
| | 11.34 (s, 1H), 10.98 (s, 1H), 9.15 |
| | (s, 1H), 8.88 (d, J = 2.2 Hz, 1H), 8.17 |
| | (s, 1H), 8.04 (dd, J = 8.4, 2.5 Hz, |
| | 1H), 7.93 (d, J = 8.3 Hz, 1H), 7.54 |
| | (dd, J = 12.5, 7.9 Hz, 2H), 7.32 (t, |
| | J = 7.9 Hz, 1H), 3.49 (s, 1H), 2.15-2.00 |
| | (m, 1H), 1.78 (s, 6H), 0.87- |
| | 0.75 (m, 4H) |
| 25 | ¹H NMR (400 MHz, DMSO-d₆) δ |
| | 11.31 (s, 1H), 11.05 (s, 1H), 9.15 |
| | (s, 1H), 8.09 (s, 1H), 7.71 (dd, J = 7.8, |
| | 1.5 Hz, 1H), 7.63 (dd, J = 8.0, |
| | 1.4 Hz, 1H), 7.37 (t, J = 7.9 Hz, 1H), |
| | 4.46 (s, 3H), 3.74 (s, 3H), 1.83 |
| | (dt, J = 7.9, 4.2 Hz, 1H), 1.33-1.17 |
| | (m, 1H), 1.07 (d, J = 6.0 Hz, 3H), |
| | 1.05-0.98 (m, 1H), 0.75-0.62 (m, 1H). |
| 26 | ¹H NMR (500 MHz, DMSO-d6) δ |
| | 11.28 (s, 1H), 10.76 (s, 1H), 9.11 |
| | (s, 1H), 8.15 (s, 1H), 7.97 (s, 1H), |
| | 7.86 (s, 1H), 7.31 (dd, J = 8.9, 5.8 |
| | Hz, 1H), 7.17 (t, J = 9.6 Hz, 1H), 3.93 |
| | (s, 3H), 2.11-2.03 (m, 1H), 0.88- |
| | 0.77 (m, 4H). |
| 27 | ¹H NMR (500 MHz, DMSO-d6) δ11.36 |
| | (s, 1H), 11.12 (s, 1H), 9.16 |
| | (s, 1H), 8.24 (d, J = 6.7 Hz, 2H), 7.99 |
| | (s, 1H), 7.30 (dd, J = 10.1, 2.7 Hz, |
| | 1H), 7.16 (dd, J = 9.8, 2.7 Hz, 1H), |
| | 3.91 (s, 3H), 3.60 (s, 3H), 2.15- |
| | 2.06 (m, 1H), 0.85 (d, J = 6.1 Hz, 4H). |
| 28 | ¹H NMR (500 MHz, DMSO-d6) δ |
| | 11.30 (s, 1H), 10.97 (s, 1H), 9.13 |
| | (s, 1H), 8.23 (s, 1H), 8.15 (s, 1H), |
| | 7.91 (s, 1H), 7.45 (d, J = 7.0 Hz, |
| | 1H), 7.29 (d, J = 7.9 Hz, 1H), 7.19 |
| | (t, J = 7.9 Hz, 1H), 3.80 (dt, J = 7.2, |
| | 3.5 Hz, 1H), 3.59 (s, 3H), 2.18-2.01 |
| | (m, 1H), 1.15-1.06 (m, 2H), |
| | 1.05-0.94 (m, 2H), 0.87-0.77 (m, 4H). |
| 29 | ¹H NMR (500 MHz, DMSO-d6) δ |
| | 11.35 (s, 1H), 11.00 (s, 1H), 9.35 |
| | (s, 1H), 9.27 (d, J = 0.9 Hz, 1H), |
| | 9.15 (s, 1H), 8.15 (s, 1H), 7.70- |
| | 7.63 (m, 2H), 7.42 (t, J = 7.9 Hz, 1H), |
| | 3.56 (s, 3H), 2.09 (br t, J = 4.6 |
| | Hz, 1H), 0.89-0.77 (m, 4H). |
| 30 | ¹H NMR (500 MHz, DMSO-d6) δ |
| | 12.32 (s, 1H), 11.30 (s, 1H), 9.84 |
| | (s, 1H), 9.22 (s, 1H), 8.45 (s, 1H), |
| | 8.07 (s, 1H), 8.03 (d, J = 5.2 Hz, |
| | 1H), 7.30 (d, J = 5.2 Hz, 1H), 3.85 |
| | (tt, J = 7.4, 3.8 Hz, 1H), 3.75 (s, |
| | 3H), 2.19-2.10 (m, 1H), 1.20-1.11 |
| | (m, 2H), 1.06-0.97 (m, 2H), |
| | 0.94-0.80 (m, 4H). |
| 31 | ¹H NMR (500 MHz, DMSO-d6) δ |
| | 12.32 (s, 1H), 11.31 (s, 1H), 9.83 |
| | (s, 1H), 9.21 (s, 1H), 8.13-7.97 (m, |
| | 2H), 7.05-7.00 (m, 1H), 3.88- |
| | 3.82 (m, 3H), 3.61-3.55 (m, 3H), |
| | 2.30-2.25 (m, 3H), 2.17-2.10 |
| | (m, 1H), 0.95-0.83 (m, 4H). |
| 32 | ¹H NMR (500 MHz, DMSO-d6) δ |
| | 12.31 (s, 1H), 11.30 (s, 1H), 9.85 |
| | (s, 1H), 9.20 (s, 1H), 8.07 (d, J = 5.2 |
| | Hz, 1H), 7.66 (s, 1H), 6.96 (d, |
| | J = 5.2 Hz, 1H), 3.83 (s, 3H), 3.53 |
| | (s, 3H), 2.34 (s, 3H), 2.18-2.06 (m, |
| | 1H), 0.95-0.83 (m, 4H). |
| 33 | ¹H NMR (500 MHz, DMSO-d6) δ |
| | 12.46 (s, 1H), 11.36 (s, 1H), 9.85 |
| | (s, 1H), 9.29 (s, 1H), 8.22 (d, J = 5.2 |
| | Hz, 1H), 8.12 (d, J = 3.1 Hz, 1H), |
| | 8.09 (d, J = 3.1 Hz, 1H), 7.76 (d, |
| | J = 5.5 Hz, 1H), 3.93 (s, 3H), 2.14 (br |
| | dd, J = 8.2, 4.0 Hz, 1H), 0.93-0.79 (m, 4H). |
| 34 | ¹H NMR (500 MHz, DMSO-d6) δ |
| | 11.34 (s, 1H), 10.97 (s, 1H), 9.17 |
| | (s, 1H), 8.60 (d, J = 1.5 Hz, 1H), 8.08 |
| | (s, 1H), 8.02 (d, J = 1.5 Hz, 1H), |
| | 7.87 (d, J = 7.9 Hz, 1H), 7.52 (d, J = |
| | 7.6 Hz, 1H), 7.36 (t, J = 7.9 Hz, |
| | 1H), 3.76 (s, 3H), 2.11-2.04 |
| | (m, 1H), 0.86-0.77 (m, 4H). |
| 35 | ¹H NMR (500 MHz, DMSO-d6) δ |
| | 11.38-11.26 (m, 1H), 10.99 (s, |
| | 1H), 9.19 (d, J = 4.6 Hz, 1H), 9.13 |
| | (s, 1H), 8.16 (s, 1H), 7.85 (d, |
| | J = 4.6 Hz, 1H), 7.72 (d, J = 7.0 Hz, |
| | 1H), 7.54 (d, J = 7.3 Hz, 1H), 7.32 |
| | (t, J = 7.8 Hz, 1H), 3.56 (s, 3H), |
| | 2.11-2.05 (m, 1H), 0.83 (br d, J = 3.1 |
| | Hz, 4H). |
| 36 | ¹H NMR (500 MHz, DMSO-d6) δ |
| | 11.32 (s, 1H), 10.99 (s, 1H), 9.34 |
| | (s, 1H), 9.13 (s, 1H), 8.96 (s, 1H), |
| | 8.17 (s, 1H), 7.50 (d, J = 7.6 Hz, |
| | 1H), 7.47 (d, J = 7.9 Hz, 1H), 7.33- |
| | 7.28 (m, 1H), 3.51 (s, 3H), 2.12- |
| | 2.05 (m, 1H), 0.89-0.78 (m, 4H). |
| 37 | ¹H NMR (500 MHz, DMSO-d6) δ |
| | 11.45-11.33 (m, 1H), 11.13 (s, |
| | 1H), 9.16 (s, 1H), 8.61 (s, 1H), 8.54 |
| | (s, 1H), 8.20 (s, 1H), 7.50 (dd, |
| | J = 9.6, 2.9 Hz, 1H), 7.33 (dd, J = 9.5, |
| | 2.7 Hz, 1H), 3.69 (s, 3H), 2.08 |
| | (quin, J = 6.0 Hz, 1H), 0.88-0.78 (m, 4H). |
| 38 | ¹H NMR (500 MHz, DMSO-d6) δ |
| | 12.42 (s, 1H), 11.33 (s, 1H), 9.86 |
| | (s, 1H), 9.60 (s, 1H), 9.23 (s, 1H), |
| | 9.11 (s, 1H), 8.16 (d, J = 5.2 Hz, |
| | 1H), 7.38 (d, J = 5.2 Hz, 1H), 3.67 |
| | (s, 3H), 2.13 (br s, 1H), 0.95-0.85 |
| | (m, 4H). |
| 39 | ¹H NMR (500 MHz, DMSO-d₆) δ |
| | 12.48 (s, 1H), 11.37 (s, 1H), 9.81 |
| | (s, 1H), 9.29 (s, 1H), 8.30 (d, J = 5.3 |
| | Hz, 1H), 7.71 (d, J = 5.2 Hz, 1H), |
| | 4.00 (s, 3H), 2.70 (s, 3H), 2.18-2.09 |
| | (m, 1H), 0.94-0.85 (m, 4H). |
| 40 | ¹H NMR (500 MHz, DMSO-d₆) δ |
| | 12.42 (br d, J = 3.1 Hz, 1H), 11.25 |
| | (br s, 1H), 9.77 (s, 1H), 9.24 (br s, |
| | 1H), 8.27 (d, J = 5.0 Hz, 1H), 7.68 |
| | (br d, J = 4.4 Hz, 1H), 3.98 (s, 3H), |
| | 2.68 (s, 3H), 1.89-1.83 (m, 1H), |
| | 1.31 (br d, J = 4.3 Hz, 1H), 1.11 |
| | (br d, J = 5.8 Hz, 3H), 1.09 (br s, 1H), |
| | 0.74 (br s, 1H). |
| 41 | ¹H NMR (500 MHz, DMSO-d₆) δ |
| | 12.53 (s, 1H), 11.39 (s, 1H), 9.83 |
| | (s, 1H), 9.32 (s, 1H), 9.13 (s, 1H), |
| | 8.33 (d, J = 5.2 Hz, 1H), 7.76 (d, |
| | J = 5.2 Hz, 1H), 4.03 (s, 3H), 2.19- |
| | 2.11 (m, 1H), 0.95-0.85 (m, 4H). |
| 42 | ¹H NMR (400 MHz, DMSO-d₆) δ |
| | 12.43 (s, 1H), 11.34 (s, 1H), 9.88 |
| | (s, 1H), 9.25 (s, 1H), 8.45 (d, J = 2.7 |
| | Hz, 1H), 8.15 (d, J = 5.3 Hz, 1H), |
| | 7.94 (s, 1H), 7.48 (d, J = 5.3 Hz, 1H), |
| | 7.13 (d, J = 2.6 Hz, 1H), 3.79 (s, |
| | 3H), 2.19-2.10 (m, 1H), 0.96-0.85 (m, 4H). |

### Biological Assays

The following assays are used to show the activity for compounds of the invention.

### IFNα-Induced STAT Phosphorylation in Human Whole Blood

After an hour long incubation with compound, human whole blood (drawn with ACD-A as anticoagulant) was stimulated with 1000 U/mL recombinant human IFNα A/D (R&D Systems 11200-2) for 15 min. The stimulation was stopped by adding Fix/Lyse buffer (BD 558049). Cells were stained with a CD3 FITC antibody (BD 555916), washed, and permeabilized on ice using Perm III buffer (BD 558050). Cells were then stained with an Alexa-Fluor 647 pSTAT5 (pY694) antibody (BD 612599) for 60 min prior to analysis on the iQue Plus. The amount of pSTAT5 expression was quantitated by median fluorescence intensity after gating on the CD3 positive population.

### Bidirectional Permeability Assay in Caco-2 Cells

### Overview

Compounds described were tested in the Caco-2 Bidirectional Permeability Assay to assess its permeability and efflux substrate potential. Compounds (at 3 μM in triplicate) were incubated with Caco-2 cells in the assay buffer at pH 7.4 (containing 0.5% bovine serum albumin [BSA]) for 2 hours at 37° C. and then was extracted for LC-MS analysis to determine its concentration in reaction mixtures and to calculate permeability coefficient, efflux ratio, and recovery.

### Materials and Methods

Caco-2 (Caucasian colon adenocarcinoma) cells were obtained from the American Type Culture Collection (Manassas, Virginia). Dulbecco's Modified Eagle's Medium (DMEM), N-2-hydroxyethylpiperazine-N′-2-ethanesulfonic acid (HEPES) buffer, nonessential amino acids, L-glutamine, penicillin-G-streptomycin, and heat-inactivated fetal bovine serum (FBS) were purchased from GIBCO/Invitrogen (Carlsbad, California). Transwell plates with 96 wells (surface area: 0.11 cm²) with a 0.4-μm pore size polycarbonate membrane and low-binding transwell cluster plates, were purchased from Sigma Aldrich (Saint Louis, Missouri). Low binding 96-well plates were purchased from Corning (Corning, New York). Modified Hank's Balanced Salt Solution (MHBSS) was prepared by adjusting Hank's Balanced Salt Solution (HBSS) with HEPES to pH 7.4. HBSS, digoxin, and bovine serum albumin (BSA) were purchased from Sigma (Saint Louis, Missouri). Filtration blocks (2 mL, 96 well) were purchased from Whatman (Freiburg, Germany). All solvents were analytical grade.

### Cell Preparation

Fourteen (14) to 28 days prior to assay, Caco-2 cells were seeded onto polycarbonate filter membranes in 96-well transwell plates at a density of 1.8×10⁵ cells/cm², approximately 2.0×10⁴ cells per well. The cells were grown in culture medium consisting of DMEM supplemented with 10% fetal bovine serum, 10 mM HEPES, 1% nonessential amino acids, 2 mM L-glutamine, 100 U/mL penicillin-G, and 100 μg/mL streptomycin. The culture medium was replaced every 3 days and the cells maintained at 37° C. in a 95% relative humidity and 5% CO₂ atmosphere. The cells were evaluated for tight junction formation just prior to assay (see Quality Control section below).

### Compound Preparation

Compounds were solubilized to 10 mM in 100% DMSO. Following visual confirmation of complete solubilization, 10 mM stock of the compounds were plated into a 96-well plate and further serially diluted in 100% DMSO to create a 100× stock concentration of 0.3 mM. Four (4) control compounds were tested alongside described compounds, they were plated at a 100× concentration of 0.3 mM in quadruplicate.

### Permeability Assessment

Compounds described were tested in triplicate in a single experiment at a final concentration of 3 μM. Cell passage used in the assay have passed QC criteria (see Quality Control section below). The study was conducted with Caco-2 cell monolayers cultured for 14 to 28 days, with cell passage numbers between 20 and 80. Assay (transport) buffer consisted of MHBSS, adjusted to pH 7.4, and 0.5% BSA. From the 100× compound plate, 8 μL of 100% DMSO stock solution of compounds were added to 800 μL assay buffer, mixed well, and filtered to remove any precipitate as a final preparation step before assay incubation. Targeted final test concentration of compounds described and control compounds was 3 μM. The filtrate represented the initial stock compound solution that was used as the donor solution for the assay (in both directions). The receiver solution was the assay buffer only.

Prior to assay execution, each cell monolayer was washed 3 times with assay buffer to remove all traces of culture media. Permeability studies were initiated by adding 100 μL assay buffer plus/minus compound to the apical transwell compartment and 200 μL assay buffer plus/minus compound to the basolateral compartment of the 96-well transwell low-binding cluster plate. For apical-to-basolateral (A→B) permeability (absorptive direction), buffer containing compounds or control compounds (1× donor solution) were placed in the apical compartments (donor wells), while buffer alone was placed in the corresponding basolateral compartments (receiver wells). For basolateral-to-apical (B→A) permeability (secretive direction), buffer containing compounds or control compounds (1× donor solution) were placed in the basolateral compartments (donor wells), while buffer alone was placed in the corresponding apical compartments (receiver wells). Transwells were then incubated for 2 hours at 37° C. in a 95% relative humidity and 5% CO₂ atmosphere. Following incubation, 75 μL was removed from each apical and basolateral compartment and transferred to 96-well low-binding plates that had been previously loaded with 75 μL/well of acetonitrile containing 250 nM propranolol, 250 nM diclofenac, and 500 nM tolbutamide as internal standards. The samples were subsequently analyzed by LC-MS/MS to determine concentrations of compounds described and control compounds.

### Analysis of Assay Samples

The concentrations of compounds described and control compounds in the assay samples were determined by LC-MS/MS. The AB Sciex 4500/5500/6500 multiplexed systems consisted of 2 sets of binary Shimadzu 20ADvp pumps with SCL-20Avp controllers for gradient elution, and LS1 autosampler, and an AB Sciex 4500/5500/6500 triple quadrupole mass spectrometer operated under electrospray ionization (ESI) mode. To obtain the optimum SRM conditions for sample analysis, MS/MS optimization for each compound was performed using DiscoveryQuant™ (AB Sciex) featuring saturation control with 5 μM standard solutions in a mixture of methanol and water (1:1, v/v) prepared from compound stock solutions. The optimization was performed using a flow injection analysis with an injection volume of 40 μL under isocratic elution of 75% of mobile phase B (0.2% formic acid in acetonitrile) and 25% mobile phase A (0.2% formic acid in water).

A 5-μL aliquot of sample was injected and then separated on a Kinetex XB-C18, 2.6 μm, 2.1×30 mm column under a gradient elution using mobile phase consisting of A (0.2% formic acid in water) and B (0.2% formic acid in acetonitrile).

TABLE A
Bidirectional Permeability in Caco-2 Cells Assay-Mobile Phase
Gradient for Sample Analysis

| Time (s) | Length (s) | Flow (mL/min) | Gradient | % A | % B |
|---|---|---|---|---|---|
| 0 | 5 | 0.7 | Step | 98 | 2 |
| 5 | 25 | 0.7 | Ramp | 2 | 98 |
| 30 | 20 | 0.7 | Step | 2 | 98 |
| 50 | 30 | 0.7 | Step | 98 | 2 |

A = 0.2% formic acid in water;
B = 0.2% formic acid in acetonitrile

DiscoveryQuant™ automatically determined the optimal ionization polarity (positive or negative), precursor and product ions, declustering potential, and collision energy for compounds described and reference compounds. The optimized SRM MS/MS conditions were used for sample analysis. The peak area ratios of compounds described or control compound to internal standard were used for quantification. The peak area ratio of compound in the dosing solution was used to determine the compound concentration in the sample.

### Data Analysis

The following results were reported for compounds described: permeability coefficient (Pc [nanometers per second]), efflux ratio and percent recovery.

The Pc value was calculated using the following equation:

$$Pc = \frac{{C}_{A t} \times {V}_{A}}{S \times {C}_{D 0} \times t}$$

### Where:

C_{At}=concentration of the test compound in acceptor well after time t,
V_{A}=volume in acceptor well,
S=surface area of the membrane (0.11 cm²),
C_{D0}=initial concentration of the test compound in donor well,
t=incubation time.

The efflux ratio was calculated as:

$$Efflux Ratio = \frac{{Pc}_{( B \to A )}}{{Pc}_{( A \to B )}}$$

Recovery (%) was calculated by expressing the total amount (nmol) of test compound present in the donor and receiver assay compartments at the end of incubation time (combined) as a fraction (percentage) of the total amount (nmol) of test compound added to the donor compartment before assay incubation. It was calculated using the following equation:

$$\% Recovery = \frac{{C}_{D t} \times {V}_{D} + {C}_{A t} \times {V}_{A}}{{C}_{D 0} \times {V}_{D}} \times 100$$

### Wherein:

C_{D0}=initial concentration of the test compound in donor well,
V_{D}=volume in donor well,
C_{Dt}=concentration in donor well after time t,
C_{At}=concentration in acceptor well after time t,
V_{A}=volume in acceptor well.

### Quality Control

The Caco-2 cells in one of the transwell plates used on the day of assay were evaluated for tight junction formation using trans-epithelial electrical resistance (TEER) measurement. TEER evaluation was performed using the EVOM resistance meter (World Precision Instruments, Sarasota, Florida). Each well of the transwell plate demonstrated a TEER value >600 Ω·cm², and the cell passage and all the plates of this plating batch were accepted for the assay.

Four (4) control compounds, with Pc values covering a range of permeability, were tested alongside the compounds described in each experiment. Acceptance criteria for this assay require that the results for the control compounds at 3 μM are within acceptable historical ranges. The acceptable ranges of the Pc values and efflux ratios observed historically for these 4 controls are shown in table B.

In these studies, the results for all control compounds were within their respective historical ranges. Thus, the assay data were accepted for the data analysis and evaluation of the compounds described bidirectional permeability in Caco-2 cells.

TABLE B
Bidirectional Permeability in Caco-2 Cells Assay-Historical Results
for Control Compounds

| Compound | Pc (A→B) (nm/s) | Pc (B→A) (nm/s) | Efflux Ratio |
|---|---|---|---|
| Digoxin | 18 ± 7 | 265 ± 74 | 14.7 |
| Nadolol | 20 ± 9 | 25 ± 11 | 1.3 |
| Atenolol | 19 ± 8 | 27 ± 10 | 1.4 |
| Verapamil | 120 ± 20 | 160 ± 40 | 1.3 |

Values are Mean ± Standard Deviation.
Pc = permeability coefficient.
A→B = apical-to-basolateral.
B→A = basolateral-to-apical.

TABLE 2
Potency and permeability data of exemplified compounds in human
whole blood assay and CACO2 permeability assay.

| Example | Human Whole blood IFNa | CACO2 (A-B) | Efflux |
|---|---|---|---|
| number | pSTAT5 IC₅₀ (μM) | (nm/s) | Ratio |
| 1 | 0.034 | 650 | 0.5 |
| 2 | 0.013 | 661 | 0.3 |
| 3 | 0.031 | 553 | 0.5 |
| 4 | 0.009 | 591 | 0.6 |
| 5 | 0.091 | 640 | 0.7 |
| 6 | 0.042 | 785 | 0.5 |
| 7 | 0.431 | 313 | 0.6 |
| 8 | 0.248 | 460 | 1.0 |
| 9 | 0.161 | 499 | 0.5 |
| 10 | 0.219 | 513 | 0.6 |
| 11 | 0.165 | 270 | 0.4 |
| 12 | 0.044 | 16 | 0.9 |
| 13 | 0.051 | 211 | 0.4 |
| 14 | 0.148 | 378 | 0.9 |
| 15 | 0.099 | 309 | 1.0 |
| 16 | 0.197 | 194 | 0.5 |
| 17 | 0.063 | 474 | 0.4 |
| 18 | 0.152 | 259 | 0.7 |
| 19 | 0.069 | 170 | 0.6 |
| 20 | 0.148 | 188 | 2.9 |
| 21 | 0.385 | 141 | 0.7 |
| 22 | 0.410 | 323 | 0.6 |
| 23 | 0.243 | 174 | 1.4 |
| 24 | 0.426 | 148 | 1.2 |
| 25 | 0.248 | 233 | 1.1 |
| 26 | 0.201 | 300 | 1.0 |
| 27 | 0.177 | 312 | 0.9 |
| 28 | 0.425 | 274 | 0.7 |
| 29 | 0.417 | 317 | 0.5 |
| 30 | 0.193 | 227 | 0.5 |
| 31 | 0.289 | 393 | 1.0 |
| 32 | 0.437 | 315 | 0.9 |
| 33 | 0.207 | 383 | 0.4 |
| 34 | 0.474 | 233 | 1.1 |
| 35 | 0.121 | 422 | 0.5 |
| 36 | 0.225 | 203 | 0.9 |
| 37 | 0.084 | 354 | 0.4 |
| 38 | 0.381 | 595 | 0.5 |
| 39 | 0.159 | 463 | 0.3 |
| 40 | 0.179 | 126 | 0.3 |
| 41 | 0.175 | 277 | 0.4 |
| 42 | 0.193 | 266 | 0.4 |
| 43 | 0.443 | 242 | 0.8 |

We claim:

1. A compound of formula I 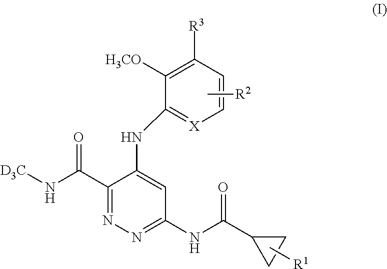
or a stereoisomer or pharmaceutically acceptable salt thereof,
wherein
X is —N— or —CH—;
R¹ is C₁₋₃ alkyl;
R² is F or C₁₋₃ alkyl;
R³ is oxadiazol, oxazol, pyridine, pyrimidine, thiadiazol, pyrazine, thiazol, pyrazol or isothiazole, all of which are substituted with 0-3 R^{3a} groups;
R^{3a} is H, F, Cl, CN, NO₂, C₁₋₄ alkyl, O—C₁₋₄ alkyl, C₃₋₆ cycloalkyl, (CH₂)ₙF, CHF₂;
hydroxy C₁₋₃ alkyl or cyano C₁₋₃ alkyl; and
n is 1 or 2.

2. The compound according to claim 1 of formula II 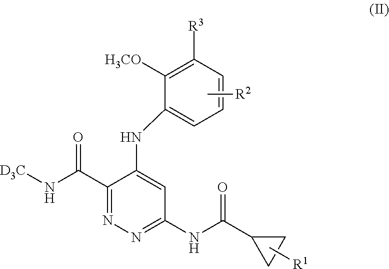
or a stereoisomer or pharmaceutically acceptable salt thereof,
wherein
R¹ is C₁₋₃ alkyl;
R² is F or C₁₋₃ alkyl;
R³ is oxadiazol, oxazol, pyridine, pyrimidine, thiadiazol, pyrazine, thiazol, pyrazol or isothiazole, all of which are substituted with 0-3 R^{3a} groups;
R^{3a} is H, F, Cl, CN, NO₂, C₁₋₄ alkyl, O—C₁₋₄ alkyl, C₃₋₆ cycloalkyl, (CH₂)ₙF, CHF₂;
hydroxy C₁₋₃ alkyl or cyano C₁₋₃ alkyl; and
n is 1 or 2.

3. The compound according to claim 1 of formula III 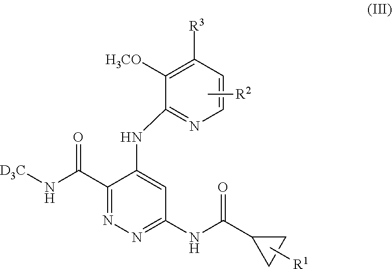
or a stereoisomer or pharmaceutically acceptable salt thereof,
wherein
R¹ is C₁₋₃ alkyl;
R² is F or C₁₋₃ alkyl;
R³ is oxadiazol, oxazol, pyridine, pyrimidine, thiadiazol, pyrazine, thiazol, pyrazol or isothiazole, all of which are substituted with 0-3 R^{3a} groups;
R^{3a} is H, F, Cl, CN, NO₂, C₁₋₄ alkyl, O—C₁₋₄ alkyl, C₃₋₆ cycloalkyl, (CH₂)ₙF, CHF₂;
hydroxy C₁₋₃ alkyl or cyano C₁₋₃ alkyl; and
n is 1 or 2.

4. A compound or a pharmaceutically acceptable salt thereof, selected from
6-cyclopropaneamido-4-({3-[5-(fluoromethyl)-1,2,4-oxadiazol-3-yl]-2-methoxyphenyl}amino)-N-(²H3)methylpyridazine-3-carboxamide
6-cyclopropaneamido-4-[(2-methoxy-3-{5-[(propan-2-yloxy)methyl]-1,2,4-oxadiazol-3-yl}phenyl)amino]-N-(²H3)methylpyridazine-3-carboxamide
6-cyclopropaneamido-4-({3-[5-(ethoxymethyl)-1,2,4-oxadiazol-3-yl]-2-methoxyphenyl}amino)-N-(²H3)methylpyridazine-3-carboxamide
6-cyclopropaneamido-4-({2-methoxy-3-[5-(2-methoxyethyl)-1,2,4-oxadiazol-3-yl]phenyl}amino)-N-(²H3)methylpyridazine-3-carboxamide
6-cyclopropaneamido-4-({3-[5-(2-fluoroethyl)-1,2,4-oxadiazol-3-yl]-2-methoxyphenyl}amino)-N-(²H3)methylpyridazine-3-carboxamide
6-cyclopropaneamido-4-{[2-methoxy-3-(1,3-oxazol-4-yl)phenyl]amino}-N-(²H3)methylpyridazine-3-carboxamide
6-cyclopropaneamido-4-({3-[5-(difluoromethyl)pyridin-2-yl]-2-methoxyphenyl}amino)-N-(²H3)methylpyridazine-3-carboxamide
6-cyclopropaneamido-4-{[2-methoxy-3-(6-methylpyrimidin-4-yl)phenyl]amino}-N-(²H3)methylpyridazine-3-carboxamide
6-cyclopropaneamido-4-{[2-methoxy-3-(1,2,4-thiadiazol-5-yl)phenyl]amino}-N-(²H3)methylpyridazine-3-carboxamide
6-cyclopropaneamido-4-{[3-(5-fluoropyrazin-2-yl)-2-methoxyphenyl]amino}-N-(²H3)methylpyridazine-3-carboxamide
6-cyclopropaneamido-4-{[3-(5-cyclopropylpyrazin-2-yl)-2-methoxyphenyl]amino}-N-(²H3)methylpyridazine-3-carboxamide
6-cyclopropaneamido-4-{[2-methoxy-3-(5-nitropyrazin-2-yl)phenyl]amino}-N-(²H3)methylpyridazine-3-carboxamide
6-cyclopropaneamido-4-{[2-methoxy-3-(3-methyl-1,2,4-thiadiazol-5-yl)phenyl]amino}-N-(²H3)methylpyridazine-3-carboxamide
6-cyclopropaneamido-4-({3-[5-(1-hydroxycyclobutyl)pyrazin-2-yl]-2-methoxy-5-methylphenyl}amino)-N-(²H3)methylpyridazine-3-carboxamide
6-cyclopropaneamido-4-({5-fluoro-3-[5-(1-hydroxycyclobutyl)pyrazin-2-yl]-2-methoxyphenyl}amino)-N-(²H3)methylpyridazine-3-carboxamide
6-cyclopropaneamido-4-{[3-(5-cyclopropylpyrazin-2-yl)-5-fluoro-2-methoxyphenyl]amino}-N-(²H3)methylpyridazine-3-carboxamide
6-cyclopropaneamido-4-{[2-methoxy-3-(2-methyl-1,3-oxazol-4-yl)phenyl]amino}-N-(²H3)methylpyridazine-3-carboxamide
6-cyclopropaneamido-4-{[3-(2,5-dimethyl-1,3-oxazol-4-yl)-2-methoxyphenyl]amino}-N-(²H3)methylpyridazine-3-carboxamide
6-cyclopropaneamido-4-({3-[5-(difluoromethyl)-1,2,4-oxadiazol-3-yl]-2-methoxyphenyl}amino)-N-(²H3)methylpyridazine-3-carboxamide
6-cyclopropaneamido-4-({4-[5-(2-hydroxypropan-2-yl)pyrazin-2-yl]-3-methoxypyridin-2-yl}amino)-N-(²H3)methylpyridazine-3-carboxamide
6-cyclopropaneamido-4-{[2-methoxy-3-(2-methyl-1,3-thiazol-4-yl)phenyl]amino}-N-(²H3)methylpyridazine-3-carboxamide
6-cyclopropaneamido-4-{[2-methoxy-3-(5-methoxypyrazin-2-yl)phenyl]amino}-N-(²H3)methylpyridazine-3-carboxamide
6-cyclopropaneamido-4-{[2-methoxy-3-(5-methylpyrazin-2-yl)phenyl]amino}-N-(²H3)methylpyridazine-3-carboxamide
4-({3-[5-(1-cyano-1-methylethyl)pyridin-2-yl]-2-methoxyphenyl}amino)-6-cyclopropaneamido-N-(²H3)methylpyridazine-3-carboxamide
4-{[2-methoxy-3-(2-methyl-2H-1,2,3,4-tetrazol-5-yl)phenyl]amino}-N-(²H3)methyl-6-[(1R,2R)-2-methylcyclopropaneamido]pyridazine-3-carboxamide
6-cyclopropaneamido-4-{[4-fluoro-2-methoxy-3-(1-methyl-1H-pyrazol-4-yl)phenyl]amino}-N-(²H3)methylpyridazine-3-carboxamide
6-cyclopropaneamido-4-{[5-fluoro-2-methoxy-3-(1-methyl-1H-pyrazol-4-yl)phenyl]amino}-N-(²H3)methylpyridazine-3-carboxamide
6-cyclopropaneamido-4-{[3-(1-cyclopropyl-1H-pyrazol-4-yl)-2-methoxyphenyl]amino}-N-(²H3)methylpyridazine-3-carboxamide
4-{[3-(5-cyanopyrazin-2-yl)-2-methoxyphenyl]amino}-6-cyclopropaneamido-N-(²H3)methylpyridazine-3-carboxamide
6-cyclopropaneamido-4-{[4-(1-cyclopropyl-1H-pyrazol-4-yl)-3-methoxypyridin-2-yl]amino}-N-(²H3)methylpyridazine-3-carboxamide
6-cyclopropaneamido-4-{[4-(1,3-dimethyl-1H-pyrazol-4-yl)-3-methoxypyridin-2-yl]amino}-N-(²H3)methylpyridazine-3-carboxamide
6-cyclopropaneamido-4-{[4-(1,5-dimethyl-1H-pyrazol-4-yl)-3-methoxypyridin-2-yl]amino}-N-(²H3)methylpyridazine-3-carboxamide
6-cyclopropaneamido-4-{[3-methoxy-4-(1,3-thiazol-2-yl)pyridin-2-yl]amino}-N-(²H3)methylpyridazine-3-carboxamide
6-cyclopropaneamido-4-{[2-methoxy-3-(1,2-thiazol-5-yl)phenyl]amino}-N-(²H3)methylpyridazine-3-carboxamide
6-cyclopropaneamido-4-{[2-methoxy-3-(1,2-thiazol-3-yl)phenyl]amino}-N-(²H3)methylpyridazine-3-carboxamide
6-cyclopropaneamido-4-{[2-methoxy-3-(1,2-thiazol-4-yl)phenyl]amino}-N-(²H3)methylpyridazine-3-carboxamide
6-cyclopropaneamido-4-{[5-fluoro-2-methoxy-3-(1,3-oxazol-4-yl)phenyl]amino}-N-(²H3)methylpyridazine-3-carboxamide
6-cyclopropaneamido-4-{[3-methoxy-4-(1,2-thiazol-4-yl)pyridin-2-yl]amino}-N-(²H3)methylpyridazine-3-carboxamide
6-cyclopropaneamido-4-{[3-methoxy-4-(3-methyl-1,2,4-thiadiazol-5-yl)pyridin-2-yl]amino}-N-(²H3)methylpyridazine-3-carboxamide
4-{[3-methoxy-4-(3-methyl-1,2,4-thiadiazol-5-yl)pyridin-2-yl]amino}-N-(²H3)methyl-6-[(1R,2R)-2-methylcyclopropaneamido]pyridazine-3-carboxamide
6-cyclopropaneamido-4-{[3-methoxy-4-(1,2,4-thiadiazol-5-yl)pyridin-2-yl]amino}-N-(²H3)methylpyridazine-3-carboxamide
6-cyclopropaneamido-4-({4-[1-(difluoromethyl)-1H-pyrazol-4-yl]-3-methoxypyridin-2-yl}amino)-N-(²H3)methylpyridazine-3-carboxamide
6-cyclopropaneamido-4-{[3-(5-ethyl-1,2,4-oxadiazol-3-yl)-2-methoxyphenyl]amino}-N-(²H3)methylpyridazine-3-carboxamide

5. A pharmaceutical composition comprising one or more compounds according to claim 1 and a pharmaceutically acceptable carrier or diluent.

6. A pharmaceutical composition comprising one or more compounds according to claim 4 and a pharmaceutically acceptable carrier or diluent.

7. A method of treating a disease, comprising administering to a patient in need of such treatment a therapeutically-effective amount of a compound according to claim 1, wherein the disease is a neurodegenerative disease.

8. The method of claim 7 wherein the neurodegenerative disease is Alzheimer's disease, Parkinson's disease, ALS, Multiple Sclerosis (RMS and/or progressive MS, including CIS, optic neuritis, neuromyelitis optica).
